(19) United States
(12) Patent Application Publication
(10) Pub. No.: US 2024/0261296 A1
(43) Pub. Date: Aug. 8, 2024

(54) THERAPEUTIC MODALITIES FOR INHIBITING PANCREATIC BETA CELL IMPAIRMENT AND TREATING DIABETES

(71) Applicants: Hadasit Medical Research Serices & Development Ltd., Jerusalem (IL); Yissum Research Development Company of The Hebrew University of Jerusalem Ltd., Jerusalem (IL)

(72) Inventors: Danielle Melloul, Jerusalem (IL); Amiram Goldblum, Tel Aviv (IL)

(73) Assignees: Hadasit Medical Research Serices & Development Ltd., Jerusalem (IL); Yissum Research Development Company of The Hebrew University of Jerusalem Ltd., Jerusalem (IL)

(21) Appl. No.: 18/554,199

(22) PCT Filed: Apr. 6, 2022
(86) PCT No.: PCT/IL2022/050358

§ 371 Date: Oct. 5, 2023

Related U.S. Application Data
(60) Provisional application No. 63/171,607, filed on Apr. 7, 2021.

(51) Int. Cl. A61K 31/5375; A61K 31/40; A61P 3/10

(52) U.S. Cl. CPC A61K 31/5375; A61K 31/40; A61P 3/10

(57) ABSTRACT

The present invention provides compositions and methods for preserving pancreatic β-cell populations and for the treatment of diabetes. In particular, embodiments of the invention relate to the use of newly identified prostaglandin receptor 3 (EP3) antagonists that are exceptionally effective in enhancing the viability and/or activity of pancreatic P cells.

### FIELD OF THE INVENTION

The invention relates to the field of diabetes management, specifically to compositions and methods for treating diabetes and preserving the viability and function of pancreatic beta cells.

### BACKGROUND OF THE INVENTION

Diabetes mellitus is a common metabolic disorder associated with abnormally high levels of glucose in the blood. There are two major types of diabetes mellitus, termed type I and type II. Type I Diabetes (T1DM, or Insulin Dependent Diabetes Mellitus) is caused by a deficiency of insulin due to an autoimmune response which leads to the destruction of the beta cells (β cells) in the Islets of Langerhans of the pancreas. An initial phase of T1DM includes an inflammation of the pancreatic islets, known as insulitis, characterized by leukocyte and macrophage infiltration into the islets followed by the actual destruction of pancreatic β cells in an autoimmune attack.

Type II Diabetes (T2DM, or Non-Insulin Dependent Diabetes Mellitus) appears to result from both a strong genetic predisposition, and environmental factors such as diet, physical activity, and age. T2DM is caused by a combination of insulin resistance and diminished β cell function. Insulin resistance is defined as the lack of sensitivity to insulin in adipose skeletal muscle, and hepatic tissue. As a result, the pancreas produces larger than normal amounts of insulin, a state defined as hyperinsulinemia. However, eventually the pancreas fails and insulin secretion levels decrease.

T2DM is a complex metabolic disorder characterized by insulin dysfunction and/or insulin insufficiency, resulting in hyperglycemia that causes diabetes-related complications in various tissues. T2DM is characterized by β-cell failure in the setting of insulin resistance. The pathophysiology of T2DM is characterized by a range of metabolic defects, imbalances, and abnormalities affecting multiple organ systems. In early stages of the disease, pancreatic β-cells adapt to insulin resistance by increasing β-cell production and mass. As the need for insulin rises, the pancreas gradually loses its ability to produce sufficient amounts of insulin to regulate blood glucose. As nutrient excess persists, hyperglycemia and elevated free fatty acids impair β-cell function followed by β-cell death.

Maintenance of beta-cell mass involves a dynamic balance between cell replication, neogenesis, and apoptosis. For patients with T2DM, there appears to be a shift toward an increase in apoptosis that outweighs cell renewal. It has been shown that a decrease in β-cell mass of about 25-60% and an increased frequency of apoptosis is found in T2DM patients (Rahier J. et al., Diabetes Obes Metab. 2008; 10 Suppl 4:32-42).

To mimic the hyperglycemic milieu pancreatic islets are subjected in diabetes, isolated human islets were exposed for a prolonged period of 10 days in high glucose concentrations to assess the detrimental effects of chronic hyperglycemia on β-cell function. It has been shown that under these conditions, several β-cell functions were impaired, including insulin secretion, content and gene expression (Marshak et al (1999), Diabetes, 48: 1230-1236).

WO2008075070 discloses sulfonamide derivatives for therapeutic use as fatty acid synthase (FAS) inhibitors. The compounds are disclosed as being useful for the treatment and prevention of obesity, and further suggested for the treatment of certain conditions that are associated with or secondary to weight gain, such as type 2 diabetes mellitus. Subsequently, Real et al. (Eur Assoc Stud Diab 2017, 408), reported that FAS inhibition has a deleterious effect on pancreatic β-cell activity. In particular, Real et al. found that the FAS inhibitor C75 (that was also identified in WO2008075070) markedly reduced glucose-induced insulin secretion and β cell electrical activity, in both chow and high-fat diet islets.

Cyclooxygenases (COX, the protein products of the prostaglandins synthase genes (PTGS)) belong to a family of enzymes that catalyze the first step in the formation of prostaglandins (PGs). Prostaglandins are important mediators of acute and chronic inflammation, development and immune functions. It appears in two isoenzymes, COX1 and COX2, which are encoded by two separate genes located at different chromosomes, differ in tissue distribution, promoter structure and non-steroid anti-inflammatory drug binding sites. COX1 is a housekeeping enzyme which is constitutively expressed in most mammalian tissues and is thought to be involved in maintaining physiological functions. In contrast, the inducible isoform, COX2, is usually expressed at low levels in most tissues and cells, but is rapidly and transiently induced by a wide range of inflammatory stimuli such as lipopolysaccharide, cytokines and chemicals.

While in most tissues, COX1 is predominantly expressed relative to COX2, in Syrian hamster (3-cell line (HIT) and human pancreatic islets under basal condition, COX2 was the dominant isoform. PGE2 is the major endogenous prostanoid derived from COX1 and COX2, it acts in an autocrine or paracrine manner and modulates inflammatory responses via a family of four G-protein-coupled receptors (GPCRs) termed EP1, EP2, EP3, and EP4. The EP subtypes exhibit differences in signal transduction, and only EP3 has been shown to couple to inhibitory G proteins (Gi) and subsequently lead to a decrease in cAMP concentration.

There are nine different splice variants of human EP3, all generated from one gene (Breyer R M. et al. Annu. Rev. Pharmacol. Toxicol 2001: 41:661-90). One splice variant is in the 3′-untranslated region; the eight expressed isoforms differ most significantly in their carboxyl-terminal domains. All eight isoforms exhibit similar ligand binding properties to PGE2. In humans, EP3 isoforms include EP₃-1, EP₃-2, EP₃-3, EP₃-4, EP₃-5, EP₃-6, EP₃-7, and EP₃-8. There are at least three isoforms in mice, namely Ep₃-α, Ep₃-β, and Ep₃-γ, wherein the gamma murine isoform is considered to be homologous to the EP₃-2 human isoform.

It has been shown that the EP3 agonist Misoprostol brings about an inhibition of glucose-induced insulin secretion in rat islets (Tran, P O et al.; Diabetes 2002: 51(6):1772-8); and mice with a knock-out of the Gi protein alpha-subunit have improved glucose tolerance due to an increase in β-cell mass (Kimple, M E et al.; JBC 2012: 287(24):20344-20355). Kimple et. al. (Diabetes 2013, 62, 1904-1912) discloses Prostaglandin E2 receptor, EP3, is induced in diabetic islets and negatively regulates glucose- and hormone-stimulated insulin secretion. Gannon, M. (Molecular metabolism 2017, 6, 548-559) discloses opposing effects of prostaglandin E2 receptors EP3 and EP4 on mouse and human β-cell survival and proliferation. Chan, P C et. al (FASEB J. 2016 30, 2282-2297) is directed to the involvement of adipocyte COX-2 and PGE2-EP3 signaling in the development of obesity-induced adipose tissue inflammation and insulin resistance.

US 2008/0200568 to Chissoe disclose a method of lowering serum blood glucose levels in a mammalian subject in need thereof, comprising administering to said subject an EP3 antagonist in an amount effective to decrease serum blood glucose levels compared to the level of serum blood glucose that would be seen in the absence of said administration. U.S. Pat. No. 9,381,176 discloses PTGER3, as a novel anti-diabetic therapeutic target. WO 2015/052610 and WO 2016/103097 disclose antagonists of prostaglandin EP3 receptor. The compounds are suggested to affect insulin secretion, and to be used in the treatment of various conditions, inter alia diabetes.

Recent publications to some of the present inventors and coworkers (Amior et al. FASEB J. 2019, Apr. 33(4):4975-4986; Melloul, 2020, Intervention in Obesity & Diabetes Vol. 3 (5):305-308) discuss the role of inter alia Cox-2 and prostaglandin E2 receptor EP3 in pancreatic β-cell function and death.

Compounds reported to be capable of blocking EP3 from responding to PGE2 or agonists thereof include e.g. DG-041, L-798106, and ONO-AE3-240, which are in development primarily as anti-thrombotics.

Traditional T2DM therapies have focused on increasing peripheral insulin sensitivity and/or activating insulin secretory pathways in the β-cell. It appears that T2DM develops once β-cells are no longer able to sustain adequate insulin secretory responses.

Because of the progressive dysfunction of the pancreatic β-cells and increasing insulin resistance over time, the need for treatments with different mechanisms or addition of medications to a regimen is becoming conventional. The clinical implications of T2DM are derived at least in part from elevated apoptosis of β-cells.

There is currently no effective cure for T1DM that restores the normal function of the pancreas, and treatment of T1DM is mainly focused on maintaining normal levels of blood sugar or glucose. T1DM is usually treated with insulin replacement therapy, for example via subcutaneous injection, along with attention to dietary management and careful monitoring of blood glucose levels using glucose meters. Other treatment options include islet cell transplantation or whole pancreas transplantation, which may restore proper glucose regulation.

There is an unmet need for novel compositions and methods of use thereof for preserving pancreatic β-cell viability and function. There is also an unmet need for improved therapeutic modalities for the treatment and management of diabetes.

### SUMMARY OF THE INVENTION

The present invention in embodiments thereof provides compositions and methods for preserving pancreatic beta-cell (0-cell) populations and for the treatment of diabetes. In some embodiments, the invention relates to newly identified prostaglandin receptor 3 (EP3) antagonists. In certain embodiments, the compositions and methods of the invention employ the use of advantageous compounds exhibiting an exceptionally superior ability to preserve or enhance the viability and/or function of pancreatic β cells. In some embodiments, provided are improved therapeutic modalities for the management of diabetes, including type II (T2DM) and type I (T1DM) diabetes, characterized by enhanced efficacy and/or safety.

The invention is based, in part, on the identification of small molecule compounds as novel therapeutic agents having EP3-inhibiting properties. Surprisingly, a number of compounds, selected from over 1.8 million candidates, were discovered to be highly effective in their ability to restore or maintain the viability and/or activity of pancreatic β cells in response to lipotoxic stimuli. Specifically, advantageous compounds were identified, capable of rescuing MIN6 rodent β cells and human islet β cells from palmitic acid-induced apoptosis, and of restoring glucose-stimulated insulin secretion and content in human islet β-cells.

Remarkably, compounds identified according to the teachings of the invention exhibited significantly enhanced efficacy in protecting β cells compared to the commercially available EP3 antagonist, L-798106, while maintaining a comparable safety profile in vivo. Further, the compounds were also found to be characterized by desirable pharmacological properties, and exhibited improved efficacy even at low concentrations. Compounds of the invention also showed high efficacy and adequate safety in an in vivo model (db/db mice in BKS background) that exhibit features of human T2DM. In particular, the compounds exerted remarkable improvement or alleviation in various parameters associated with the development of β cell pathologies in db/db mice, including with respect to glycemia, fasting blood glucose, glucose tolerance and insulin resistance. In addition, administration of the compounds was accompanied by significant reduction in blood triglyceride levels in db/db mice.

According to some embodiments, provided are compositions and methods for the treatment and management of diabetes. In various embodiments, the compositions and methods of the invention are used for preventing or inhibiting loss of pancreatic β cell mass and/or activity in a patient suffering from, or prone to developing, diabetes (e.g. T2DM). In some embodiments, provided are compositions and methods for preserving a pancreatic β cell population (e.g. in vivo, ex vivo or in vitro). In various embodiments, the compositions and methods of the invention are capable of reducing or diminishing β-cell apoptosis, increasing the survival of β-cells and/or increasing or maintaining β-cell mass. In some embodiments, the compositions and methods of the invention are used for preventing or reducing the level or incidence of hyperglycemia. In other embodiments, the compositions and methods of the invention are used for enhancing or at least preserving the glucose-induced insulin secretion capacity of pancreatic β cells. In other embodiments, the compositions and methods of the invention are used for reducing insulin resistance. In other embodiments, the compositions and methods of the invention are used for reducing or preventing lipotoxicity-induced β cell damage. In other embodiments, the compositions and methods of the invention are used for preventing or delaying the onset or progression of diabetes in a subject in need thereof (e.g. a pre-diabetic subject, or a subject afflicted with dyslipidemia and hyperglycemia). In some embodiments, the compositions and methods of the invention are used in the maintenance of pancreatic β cells or pancreatic islets in culture, e.g. for preparing cell compositions for transplantation.

In various embodiments, the compositions and methods of the invention employ the use of compounds represented by a formula selected from the general formulae I, II and III, as follows.
(i) Formula I, or a salt thereof:

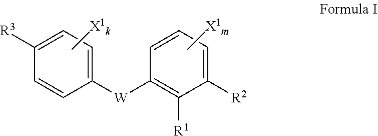

wherein
W is —NHSO—, wherein n is 1 or 2;
one of R¹ and R² is CONR⁴R⁵, and the other one of R¹ and R² is hydrogen, wherein each
one of R⁴ and R⁵ is independently selected from the group consisting of H, alkyl, aryl, arylalkyl, cycloalkyl and haloalkyl, or wherein R⁴ and R⁵ together with the nitrogen atom to which they are bound form a ring, wherein the formed ring is unsubstituted or substituted with at least one alkyl, halogen, or haloalkyl;
each one of X¹ and Y¹ is independently selected from the group consisting of a halogen and an alkyl;
each one of m and k is 0, 1 or 2; and
R³ is selected from hydrogen and an alkyl group;
(ii) Formula II, an ester or a salt thereof:

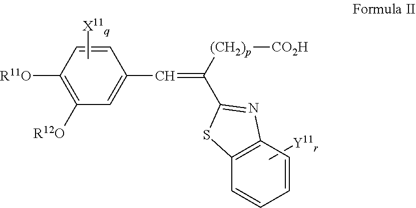

wherein
R¹¹ is CH₂R¹³, wherein R¹³ is selected from the group consisting of hydrogen, an alkyl and an aryl;
R¹² is selected from the group consisting of hydrogen and an alkyl;
each one of X¹¹ and Y¹¹ is independently selected from the group consisting of a halogen and an alkyl; and
each one of p, r and q is 0, 1 or 2; and
(iii) Formula III, an ester or a salt thereof:

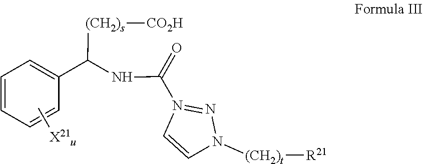

wherein
u is 1 or 2;
each X²¹ is selected from the group consisting of OH, OR²² and halogen, wherein R²² is selected from the group consisting of unsubstituted alkyl, unsubstituted aryl, haloalkyl, alkoxyalkyl, hydroxyalkyl and haloaryl;
each one of s and t is independently 0, 1 or 2; and
R²¹ is selected from the group consisting of unsubstituted aryl, haloaryl, alkoxyaryl, alkylaryl, unsubstituted heteroaryl, haloheteroaryl, alkoxyheteroaryl and alkylheteroaryl.

These compounds, including pharmaceutically acceptable salts and esters thereof, are further referred to herein as the compounds of the invention. Exemplary and particularly advantageous compounds of the invention are further identified and described below.

In some embodiments, the compounds of the invention are EP3 antagonists or inhibitors. In some embodiments, the compounds of the invention inhibit EP3-mediated signaling in pancreatic β cells. In some embodiments, the compounds of the invention inhibit PGE₂-induced signaling mediated by EP3. In some embodiments, the compounds of the invention inhibit EP3-mediated cellular function in pancreatic β cells. In various embodiments, the cellular functions include, but are not limited to, β cell apoptosis and/or impaired insulin secretion, synthesis and/or storage by β-cells. In some embodiments, the cellular function or signaling is induced or enhanced in the presence of free fatty acids (FFA), e.g. palmitate. In a particular embodiment, said compounds inhibit palmitate-induced apoptosis in human islet β cells. In some embodiments, the compounds of the invention inhibit signaling or activity mediated by the gamma isoform of EP3 in murine pancreatic β cells and/or of the EP₃-2 isoform in human pancreatic β cells. In some embodiments, the compounds of the invention are selective antagonists of EP3. In some embodiments, the compounds of the invention are selective antagonists of EP3 gamma. Each possibility represents a separate embodiment of the invention.

In one aspect, there is provided a pharmaceutical composition for use in treating or preventing the progression of diabetes in a human subject in need thereof, the composition comprising a compound represented by a formula selected from the group consisting of Formulae I, II and III, esters and salts thereof. In another aspect, there is provided a method for treating or preventing the progression of diabetes in a human subject in need thereof, comprising administering to the subject a pharmaceutical composition comprising a compound represented by a formula selected from the group consisting of Formulae I, II and III, esters and salts thereof.

In one embodiment, the compound is represented by Formula I or a salt thereof. In another embodiment, the compound is represented by Formula I-1, or a salt thereof:

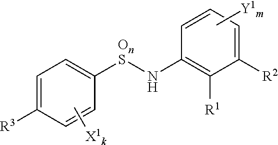

wherein R¹, R², R³, X¹, Y¹, n, m and k are as defined herein with respect to Formula I.

In another embodiment, R² is CONR⁴R⁵, and R¹ is hydrogen. In another embodiment, m is 0 and n is 2. In another embodiment, R⁴ and R⁵ together with the nitrogen atom to which they are bound form a non-aromatic N-heterocycle wherein the non-aromatic N-heterocycle is unsubstituted or substituted with one or more alkyl, halogen, or haloalkyl. In another embodiment R³ is a C₁-C₄ alkyl.

According to specific embodiments, the compound is selected from the group consisting of Compound 1a, Compound 1b, Compound 1c and salts thereof, wherein each possibility represents a separate embodiment of the invention:

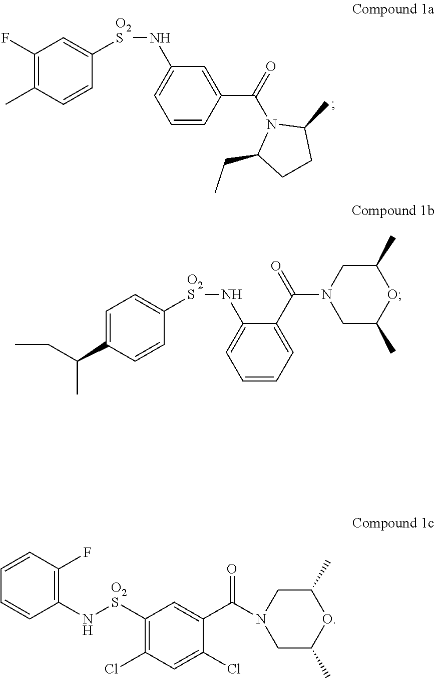

In another embodiment, said compound is Compound 1a. In another embodiment said compound is Compound 1b.

In another embodiment, said compound is represented by Formula IIa, an ester or a salt thereof:

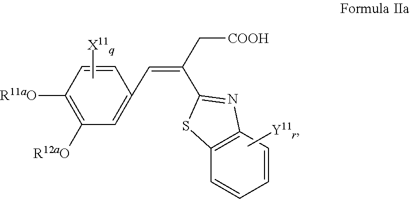

wherein
R^{11a} is CH₂R^{13a}, wherein R^{13a} is selected from the group consisting of hydrogen, an alkyl and an aryl;
R^{12a} is selected from the group consisting of hydrogen and an alkyl;
each one of X¹¹ and Y¹¹ is independently selected from the group consisting of a halogen and an alkyl; and
each one of p, r and q is 0, 1 or 2.

In another embodiment, R^{13a} is a phenyl group substituted with one substituent selected from the group consisting of alkyl, hydroxy, haloalkyl and halogen; wherein R^{12a} is a C₁-C₄ alkyl, and wherein q is 0 and r is 0. In another embodiment, said compound is represented by Formula IIb, an ester or a salt thereof:

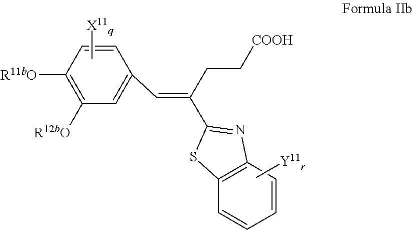

wherein
R^{11b} is CH₂R^{13b}, wherein R^{13b} is selected from the group consisting of hydrogen, an alkyl and an aryl;
R^{12b} is selected from the group consisting of hydrogen and an alkyl;
each one of X¹¹ and Y¹¹ is independently selected from the group consisting of a halogen and an alkyl; and
each one of p, r and q is 0, 1 or 2.

In another embodiment, said compound is selected from the group consisting of Compound 2a, Compound 2b, Compound 2c esters and salts thereof:

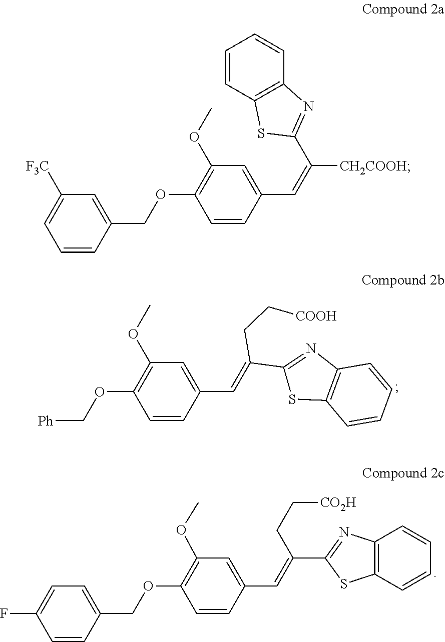

In another embodiment, said compound is Compound 3, or an ester or a salt thereof:

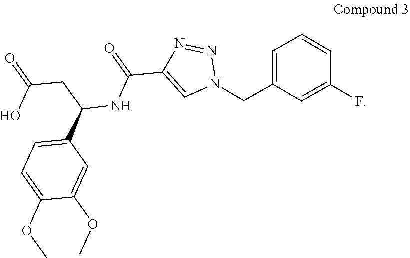 [SMILES: COc1ccc([C@@H](CC(=O)O)NC(=O)c2cn(Cc3cccc(F)c3)nn2)cc1OC]

In another embodiment, said compound is a prostaglandin receptor 3 (EP3) antagonist. In another embodiment, said compound inhibits palmitate-induced apoptosis in human islet β cells. In another embodiment, the diabetes is type II diabetes mellitus (T2DM). In another embodiment, the diabetes is type I diabetes mellitus (T1DM). In another embodiment, the diabetes is characterized by EP3 overexpression in pancreatic β cells. In another embodiment, said subject is diagnosed with chronic hyperglycemia and dyslipidemia. In another embodiment, said subject is a non-obese human subject.

In another embodiment the composition is formulated for oral administration. In another embodiment the composition is administered orally. In another embodiment, the composition is used (or administration is performed) so as to prevent or inhibit loss of pancreatic β cell mass and/or activity in said subject. In another embodiment, the composition is used (or administration is performed) so as to prevent or reduce the level or incidence of hyperglycemia in said subject. In another embodiment, the composition is used (or administration is performed) so as to prevent or reduce lipotoxicity-induced β cell damage. In another embodiment, the composition is used (or administration is performed) so as to enhance glucose-induced insulin secretion by pancreatic β cells in said subject.

In another aspect, there is provided a method for preserving or promoting the viability of pancreatic β cells, comprising contacting a population (or preparation) of the β cells with a compound represented by a formula selected from the group consisting of Formulae I, II and III, esters and salts thereof. In another aspect, the invention relates to a compound represented by a formula selected from the group consisting of Formulae I, II and III, esters and salts thereof, for use in preserving or promoting the viability of pancreatic β cells, wherein a population (or preparation) of the β cells is contacted with the compound.

In one embodiment, the contacting is performed ex vivo. In another embodiment, the contacting is performed in vitro. In another embodiment, said contacting is performed in vivo. In another embodiment said β cells are characterized by EP3 overexpression. In another embodiment said β cells are obtained from a healthy human donor. In various embodiments, the cell population (or preparation) is selected from the group consisting of: purified primary β cells, β cell lines, genetically modified β cells, primary pancreatic islet cells, and fetal pancreatic islet cells, wherein each possibility represents a separate embodiment of the invention. In another embodiment, said contacting is performed so as to prevent or reduce lipotoxicity-induced β cell damage. In another embodiment, said compound is used at an amount sufficient to prevent or reduce lipotoxicity-induced β cell damage.

In another embodiment the β cells are from a human subject having a pancreatic β cell disorder selected from the group consisting of: pancreatic β cell failure, T2DM, T1DM, pre-diabetes, and insulin resistance, or from a human subject diagnosed with chronic hyperglycemia and dyslipidemia. In another embodiment said subject further receives a pancreatic β cell transplantation, a pancreatic islet transplantation or a pancreatic transplantation. In another embodiment, said subject is a non-obese human subject.

In another embodiment said compound is represented by Formula I-1 as defined herein, or a salt thereof. In another embodiment said compound is represented by Formula IIa as defined herein, or an ester or a salt thereof. In another embodiment, said compound is represented by Formula IIb as defined herein, or an ester or a salt thereof. In another embodiment the compound is selected from the group consisting of compounds Compound 1a, Compound 1b, Compound 1c, Compound 2a, Compound 2b, Compound 2c, and Compound 3 as set forth above, esters and salts thereof. In another embodiment said compound is an EP3 antagonist (e.g. a selective EP3 antagonist). In another embodiment, said compound inhibits palmitate-induced apoptosis in human islet β cells.

In another embodiment, there is provided a cell composition for pancreatic β cell transplantation or pancreatic islet transplantation, comprising the β cell population (or preparation) that has been contacted with said compound. In various embodiments, the β cell population (or preparation) is selected from the group consisting of purified primary β cells, β cell lines, genetically modified β cells, primary pancreatic islet cells, and fetal pancreatic islet cells, wherein each possibility represents a separate embodiment of the invention.

In yet another aspect, there is provided a pharmaceutical composition comprising a pharmaceutical grade purity of one or more compounds of the invention, and a pharmaceutically acceptable carrier. In some embodiments, the compound is selected from the group consisting of Compound 1a, Compound 1b, Compound 1c, Compound 2a, Compound 2b, Compound 2c, and Compound 3 as set forth herein, esters and salts thereof. In another embodiment, the composition comprises at least one of Compound 1a, Compound 1b and salts thereof. In another embodiment, the composition comprises at least one of Compounds 2a, 2b, 2c, esters and salts thereof.

In another embodiment, said composition is for use as a medicament. In another embodiment, said composition is for use in the preparation of a medicament. In various embodiments, said medicament or composition is for the treatment of diabetes (e.g. T1DM or T2DM) in a human subject in need thereof, or for preserving or promoting the viability of pancreatic β cells, wherein each possibility represents a separate embodiment of the invention. In another embodiment, there is provided a method of treating or preventing the progression of diabetes in a human subject in need thereof, comprising administering to the subject said pharmaceutical composition, thereby treating or preventing the progression of diabetes in said subject. In another embodiment, there is provided a method of preserving or promoting the viability of pancreatic β cells in a human subject in need thereof, comprising administering to the subject said pharmaceutical composition, thereby preserving or promoting the viability of pancreatic β cells. In various other embodiments, the subject to be treated is as disclosed herein, wherein each possibility represents a separate embodiment of the invention.

Other objects, features and advantages of the present invention will become clear from the following description and drawings.

### BRIEF DESCRIPTION OF THE DRAWINGS

FIG. 1. Bar graphs showing the effects of the indicated compounds compared to the commercial EP3 antagonist L-798106 (Dark) in MIN6 cells. The graphs represent the % of rescue of palmitate-treated MIN6 cells in the presence of the designated compound as measured by western blot analysis for cleaved caspase 3 (CC3-apoptotic marker). The densitometry values of CC3 normalized to tubulin were calculated and the average of n=3-6±SEM for each sample.

FIG. 2. Bar graphs showing the effects of the tested compounds compared to the commercial antagonist L-798106 (dark column) in MIN6 cells. The graph represents the % of rescue of palmitate-treated cells MIN6 cells in the presence of the designated compounds as measured by FACS for the apoptotic subG1 population of cells. The average of n=4-6±SEM for each tested compound is presented.

FIGS. 3A-3B. Screening of compounds and their ability to rescue human islets from palmitic acid-induced apoptosis. FIG. 3A—donor 1, FIG. 3B—donor 2.

FIG. 4. The effect of the C5, C13 compounds on MIN6 β-cell apoptosis. MIN6 cells were exposed to 250 nM of the tested compound in the presence of palmitic acid (PA) for 24 h, C5 (C5+PA), C13 (C13+PA), PA (only palmitic acid). Caspase 3/7 enzymatic activity was measured and the results represent the average ±SEM of triplicates or quadruplicates.

FIGS. 5A-5C. Caspase3/7 enzymatic activity in human islets (donor 1) exposed to palmitic acid and the different compounds (C5, C13 and L-798106) and at different concentrations FIG. 5A—5 μM, FIG. 5B—2.5 μM and FIG. 5C—0.5 μM.

FIGS. 6A-6B. Beneficial effect of C5 on palmitic acid-induced downregulation of Insulin1 (FIG. 6A) and insulin 2 (FIG. 6B) gene expression. MIN6 cells were exposed to PA in the presence or absence of the EP3 antagonist L-798106 (L) or the C5 compound (10 μM). RNAs were analyzed by real-time PCR and the expression normalized to GAPDH.

FIGS. 7A-7B. The effect of the compounds on insulin secretion and content in human islet β-cells (Donor 1). Pancreatic islets from human donor 1 (“HI-1”) were exposed to the compounds L-798106 (“L”), C5, C13, (5 M) in the absence of palmitic acid (“C”) or the presence of palmitic acid 0.3 mM (“PA”) for 48 hr. FIG. 7A—Islets were then incubated in Basal (3.3 mM, empty bars) then Stimulated (16.7 mM, black bars) glucose concentrations, and secreted insulin was measured using ELISA. FIG. 7B—Insulin content from treated cells collected and measured.

FIGS. 8A-8B. The effect of the compounds on glucose stimulated insulin secretion in human islet β-cells (Donor 2—“HI-2”), as described in FIGS. 7A-7B. FIG. 8A—Glucose stimulated-insulin secretion, FIG. 8B—Insulin content.

FIGS. 9A-9D. Glucose concentrations measured in blood obtained from the tail vein of C57Bl/6 mice (n=8/group) before gavage (t0), at time 5 h (t5) after each gavage (5G1-5G2-5G3) and 24 h (t24) after the last gavage with: FIG. 9A—saline/DMSO, FIG. 9B—C5, FIG. 9C—C13, FIG. 9D—L-798106.

FIGS. 10A-10B. Blood insulin concentrations measured in blood obtained from the tail vein of C57Bl/6 mice. FIG. 10A—oral administration, (n=8/group) 24 h (t24) after the third gavage with saline/DMSO (Control Saline); 3 mg/kg/day Compound C5; Compound C13 or L-798106 (total 9 mg/kg) using ELISA mouse insulin kit (Mercodia). FIG. 10B—intraperitoneal (IP) administration, single dose of 3 mg/kg of the compounds and control as described in FIG. 10A.

FIGS. 11A-11C. Liver parameters measured in sera obtained from mice 24 h after the third gavage with saline/DMSO (empty bars); or 3 mg/kg/day of Compound C5, C13, or L-798106. FIG. 11A—Albumin (g/L); FIG. 11B—aspartate transaminase (AST); FIG. 11C—alanine transaminase (ALT). are presented. Values (±SEM), statistical analysis between each treated-group compared to Saline-control was performed for each test and all p-values were >0.05.

FIGS. 12A-12B. Kidney parameters measured in sera obtained from mice 24 h after the third gavage with saline/DMSO (Control Saline, empty bars); or 3 mg/kg/day of Compound C5, C13 or L-798106. FIG. 12A—Creatinine (M/L); FIG. 12B—Urea (mM/L). Values (±SEM), statistical analysis of the treated-groups vs saline-control shows a p-value >0.05 for each test.

FIGS. 13A-13D. Glucose concentrations measured in blood obtained from the tail vein of C57Bl/6 mice (n=12/group) before injection (t0), at time 5 h (t5) and 24 h (t24) after injection of: FIG. 13A-saline/DMSO (Control Saline, empty bars); FIG. 13B—C5, FIG. 13C—C13 FIG. 13D—L-798106. Values (±SEM) of glucose concentrations are presented. The statistical analysis was performed using one-way Anova: All p-values >0.05.

FIGS. 14A-14C. Liver parameters measured in the serum obtained from mice 24 h after injection of saline/DMSO (empty bars); or Compound C5, C13, or L-798106. Values (±SEM) of Albumin (g/L, FIG. 14A); aspartate transaminase (AST, FIG. 14B) and alanine transaminase (ALT, FIG. 14C) are presented. Statistical analysis comparing the experimental groups and the Saline-control group was performed for each test and all p-values were >0.05; except *, P=0.03.

FIGS. 15A-15B. Kidney parameters measured in serum obtained from mice 24 h after injection of saline/DMSO (Control Saline, empty bars); or Compound C5, C13 or L-798106. FIG. 15A—Creatinine (μM/L); FIG. 15B—Urea (mM/L). Values (±SEM), statistical analysis of the experimental-groups vs saline-control shows a p-value >0.05 for each test.

FIGS. 16A-16B. EP3γ is selectively upregulated by palmitate and specific EP3γ siRNA rescue MIN6 cells from palmitate-mediated apoptosis. MIN6 cells were transfected with siRNAs to total EP3, specific EP3γ subtype or negative control siRNA by electroporation using Amaxa nucleofector reagent and Amaxa nucleoporator. After 24 h, the cells were treated with 0.3 mM palmitate for an additional 24 hrs. FIG. 16A—Real time PCR analysis of EP3 mRNA subtypes expression levels normalized to GAPDH. FIG. 16B—Western blot analysis of cleaved Caspase 3, and α-tubulin expression levels.

FIGS. 17A-17D depict graphic representation of random blood glucose levels in control and db/db diabetic mice receiving vehicle or the EP3 inhibitors C5, C13 and L-798106. FIG. 17A—weekly random blood glucose levels in control mice; FIG. 17B—average random blood glucose levels in control mice (calculated using the values obtained weekly from n=10-15 mice over a period of 8 weeks); FIG. 17C—weekly random blood glucose levels in db/db mice; FIG. 17D—average random blood glucose levels in db/db mice.

FIGS. 18A-18D depict graphic representation of the effect of the compounds on glucose tolerance (IPGTT) in diabetic db/db and control mice following 8 weeks of oral administration of vehicle or the EP3 inhibitors C5, C13 and L-798106. Blood glucose levels are presented for both the control and db/db mice in FIGS. 18A and 18C respectively, while the area under the curve (AUC) corresponding to the graphs are presented for both the control and db/db mice in FIGS. 18B and 18D respectively.

FIGS. 19A-19D depict graphic representation of Insulin Tolerance Test (ITT) results, in correlation to the effect of the compounds on insulin tolerance in db/db and control mice, following 8 weeks of oral administration of vehicle or the EP3 inhibitors C5, C13 and L-798106 and 0.75 U/kg Insulin injection. Blood glucose levels are presented for both the control and db/db mice in FIGS. 19A and 19C respectively, while the AUC corresponding to the graphs are presented for both the control and db/db mice in FIGS. 19B and 19D respectively.

FIGS. 20A-20D depict graphic presentation fasting blood glucose (FBG) levels in db/db and control mice following oral administration of vehicle or the EP3 inhibitors C5, C13 and L-798106. Results after 4-weeks treatment of control mice (FIG. 20A) and of db/db mice (FIG. 20B). Results after 8-weeks treatment of control mice (FIG. 20C) and of db/db mice (FIG. 20D).

FIG. 21 depicts blood insulin levels assessed by measuring the serum circulating insulin C-peptide using ELISA, following 8 weeks of oral administration of vehicle or the EP3 inhibitors C5, C13 and L-798106 in db/db and control mice.

FIGS. 22A-22B depict blood liver parameters in sera obtained following 8 weeks of oral administration of vehicle or the EP3 inhibitors C5, C13 and L-798106 in db/db mice. FIG. 22A—Albumin levels, FIG. 22B—ALT levels.

FIGS. 23A-23B depict blood kidney parameters in sera obtained following 8 weeks of oral administration of vehicle or the EP3 inhibitors C5, C13 and L-798106 in db/db mice. FIG. 23A—Creatinine (CRE) levels, FIG. 23B—Urea levels.

FIGS. 24A-24B depict blood lipid profiles in sera obtained following 8 weeks of oral administration of vehicle or the EP3 inhibitors C5, C13 and L-798106 in db/db mice. FIG. 24A—triglyceride (TGs) levels, FIG. 24B—total cholesterol levels.

FIGS. 25A-25C depict graphic presentation of random blood glucose levels in control and db/db diabetic mice receiving vehicle or the EP3 inhibitors C15, C17 and C20. FIG. 25A—averaged random blood glucose levels in db/db mice including mice treated with the C15 compound (calculated using the values obtained from n=5-13 mice following 8 weeks gavage), which appears in the graph as C #15. FIG. 25B—weekly random blood glucose levels in control mice; FIG. 26C—weekly random blood glucose levels in db/db mice.

FIGS. 26A-26D depict graphic presentation of the effect of the compounds on glucose tolerance (IPGTT) in diabetic db/db and control mice following 8 weeks of oral administration of vehicle or the EP3 inhibitors C17 and C20 and L-798106. Blood glucose levels are presented for both the control and db/db mice in FIGS. 26A and 26C respectively, while the area under the curve (AUC) corresponding to the graphs are presented for both the control and db/db mice in FIGS. 26B and 26D respectively.

FIGS. 27A-27D depict graphic presentation of ITT results, in correlation to the effect of the compounds on insulin tolerance in db/db and control mice, following 8 weeks of oral administration of vehicle or the EP3 inhibitors C17 and C20 and L-798106, and 0.75 U/kg Insulin injection. Blood glucose levels are presented for both the control and db/db mice in FIGS. 27A and 27C respectively, while the AUC corresponding to the graphs are presented for both the control and db/db mice in FIGS. 27B and 27D respectively.

FIG. 28 depicts blood insulin levels assessed by measuring the serum circulating insulin C-peptide using ELISA. following 8 weeks weeks of oral administration of vehicle or the EP3 inhibitors C17 and C20 and L-798106 in db/db and control mice.

FIGS. 29A-29D depict graphic presentation FBG levels in db/db and control mice following oral administration of vehicle or the EP3 inhibitors C17 and C20 and L-798106. Results for 4-weeks treatment of control mice and of db/db mice are shown in FIGS. 29A and 29B, respectively. Results for 8-weeks treatment of control mice and of db/db mice are shown in FIGS. 29C and 29D, respectively.

### DETAILED DESCRIPTION OF THE INVENTION

The present invention provides compositions and methods for preserving pancreatic beta-cell ((3-cell) populations and for the treatment of diabetes. In particular, embodiments of the invention relate to the use of newly identified prostaglandin receptor 3 (EP3) antagonists that are exceptionally effective in enhancing the viability and/or activity of pancreatic β cells. Using extensive in silico, in vitro, and ex vivo analyses, a number of selected compounds were identified, having exceptional therapeutic properties. Remarkably, compounds identified as disclosed herein exhibited enhanced efficacy in preserving the viability and function of pancreatic β cells compared to commercially available EP3 antagonist, while maintaining a comparable safety profile in vivo.

As further disclosed herein, the compounds of the invention are capable not only of preventing pancreatic β cells demise, but also of restoring β cell loss of function, thereby reversing pathological processes associated with the development of diabetes. Accordingly, without being bound by a specific theory or mechanism of action, the compositions and methods of the invention may be used in the treatment of prediabetic and newly diagnosed diabetic patients, as well as advanced stage diabetic patients for which current therapeutic options are limited.

In one aspect, there is provided a pharmaceutical composition for use in treating or preventing the progression of diabetes in a human subject in need thereof, the composition comprising a compound represented by a formula selected from the group consisting of:
(i) Formula I, or a salt thereof:

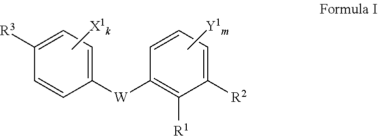

wherein W is —NHSOₙ—, wherein n is 1 or 2; one of R¹ and R² is CONR⁴R⁵, and the other one of R¹ and R² is hydrogen, wherein each one of R⁴ and R⁵ is independently selected from the group consisting of H, alkyl, aryl, arylalkyl, cycloalkyl and haloalkyl, or wherein R⁴ and R⁵ together with the nitrogen atom to which they are bound form a ring which is unsubstituted or substituted with one or more substituents, each selected from the group consisting of alkyl, halogen, or haloalkyl; each one of X¹ and Y¹ is independently selected from the group consisting of a halogen and an alkyl; each one of m and k is 0, 1 or 2; and R³ is selected from hydrogen and an alkyl group;
(ii) Formula II, an ester or a salt thereof:

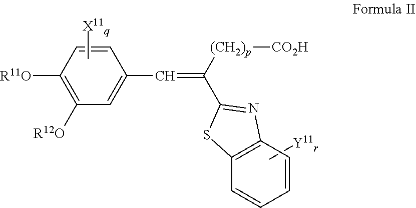

wherein R¹¹ is CH₂R¹³, wherein R¹³ is selected from the group consisting of hydrogen, an alkyl and an aryl; R¹² is selected from the group consisting of hydrogen and an alkyl; each one of X¹¹ and Y¹¹ is independently selected from the group consisting of a halogen and an alkyl; and each one of p, r and q is 0, 1 or 2; and
(iii) Formula III, an ester or a salt thereof:

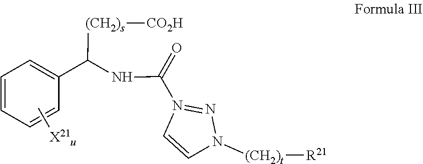

wherein u is 1 or 2; each X²¹ is selected from the group consisting of OH, OR²² and halogen, wherein R²² is selected from the group consisting of unsubstituted alkyl, unsubstituted aryl, haloalkyl, alkoxyalkyl, hydroxyalkyl and haloaryl; each one of s and t is independently 0, 1 or 2; and R²¹ is selected from the group consisting of unsubstituted aryl, haloaryl, alkoxyaryl, alkylaryl, unsubstituted heteroaryl, haloheteroaryl, alkoxyheteroaryl and alkylheteroaryl.

In another aspect, the invention provides a method for treating or preventing the progression of diabetes in a human subject in need thereof, comprising administering to the subject a pharmaceutical composition comprising a compound represented by a formula selected from the group consisting of Formulae I, II and III as defied herein, esters and salts thereof.

In another aspect, there is provided a method for preserving or promoting the viability of pancreatic β cells, comprising contacting a population (or preparation) of the β cells with a compound represented by a formula selected from the group consisting of Formulae I, II and III, esters and salts thereof. In another aspect, the invention relates to a compound represented by a formula selected from the group consisting of Formulae I, II and III, esters and salts thereof, for use in preserving or promoting the viability of pancreatic β cells, wherein a population (or preparation) of the β cells is contacted with the compound.

In yet another aspect, there is provided a pharmaceutical composition comprising a pharmaceutical grade purity of one or more compounds of the invention, and a pharmaceutically acceptable carrier. In some embodiments, the compound is selected from the group consisting of Compound 1a, Compound 1b, Compound 1c, Compound 2a, Compound 2b, Compound 2c, and Compound 3 as set forth below, esters and salts thereof:

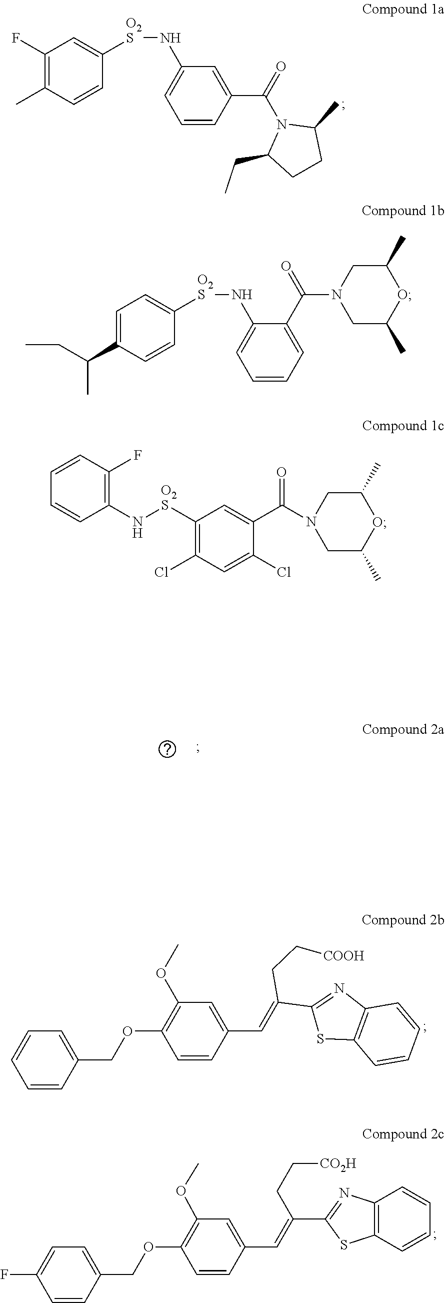

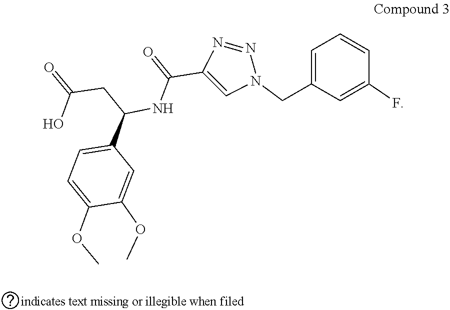 [SMILES: COc1ccc([C@@H](CC(=O)O)NC(=O)c2cn(Cc3cccc(F)c3)nn2)cc1OC]

According to some embodiments, said composition further comprising a pharmaceutically acceptable carrier, excipient or diluent.

### Prostaglandin EP₃ Receptor Activity and Modulation

The invention in aspects and embodiments thereof employs the use of compounds identified herein as EP3 antagonists or inhibitors. In some embodiments, the compounds of the invention inhibit EP3-mediated signaling in pancreatic β cells. In some embodiments, the compounds of the invention inhibit PGE₂-induced signaling mediated by EP3.

E prostanoid 3 (EP3) receptor, also known as prostaglandin EP3 receptor or EP3, is a receptor for prostaglandin E2 (PGE₂) encoded by the PTGER3 gene. The human PTGER3 codes for at least 8 different functional isoforms or variants of the EP3 receptor (namely EP3-1, EP3-2, EP3-3, EP3-4, EP3-5, EP3-6, EP3-7, and EP3-8), generated by alternative splicing. For example, information on human PTGER3 and transcripts thereof may be found at Gene ID: 5733; an exemplary sequence of human EP3-2 transcript (EP3-II or variant 5) is provided in Accession no. NM_198715.3; and an exemplary sequence of murine EP-3 gamma is provided in Accession no. D17406.1, all incorporated herein by reference.

EP3-mediated signaling includes coupling of the EP3 receptor to a Gi-type G protein, which leads to a decrease of intracellular cAMP levels and increased intracellular calcium levels. PGE₂-induced signaling mediated by EP3 refers to such a signaling cascade that is initiated or upregulated by PGE2-EP3 binding. In comparison, PGE₂-induced signaling mediated by other prostaglandin receptors results in distinct and typically opposing effects (e.g. exhibited as enhanced intracellular cAMP levels). EP3 mediated signaling may be inhibited or downregulated by EP3 antagonists and inhibitors, in particular in pancreatic β cells, as described in further detail herein.

As used herein, the term antagonist (e.g. EP3 antagonist) refers to a compound that specifically binds to the target molecule (e.g. receptor), and downregulates its activity (e.g. by blocking the ligand-binding site or allosterically). In particular, EP3 antagonists inhibit EP3 activity including PGE₂-induced signaling mediated by EP3. The antagonist can act on any of the known splice variant forms of EP3 receptor or isoforms.

Evaluating or quantifying EP3 signaling as well as the efficacy and specificity of EP3 antagonists and inhibitors may be performed by various methods known in the art. Competition of PGE₂ binding, for example can be determined using a ^{3H}PGE₂ binding assay. Cell membranes can be prepared and incubated with tritiated PGE₂. Nonspecific binding can be determined using excess unlabeled PGE₂. Specific binding can be calculated by subtracting the nonspecific binding from total binding. Alternatively or additionally, intracellular free Ca²⁺ concentration ([Ca²⁺]ᵢ) can be determined. The [Ca²⁺], can be measured as described in Miwa, et al (1988) J. Neurochem. 50, 1418-1424. Fluorescence can be measured at excitation wavelengths of 340 and 380 nm and an emission wavelength of 510 nm, with a fluorescence spectrometer. The [Ca²⁺]ᵢ can be calculated from cellular fura-2 fluorescence. Alternatively or additionally, cAMP formation can be measured as reported in Okuda-Ashitaka, et al. (1990) Eicosanoids 3, 213-218. The cAMP formed can be measured using ELISA such as an ENCO cAMP assay kit. Antagonists will have at least a 10% reducing effect on a measured parameter. Preferred antagonists will have at least a 20, 25, 30, 45, 50, 55, 60, 65, 70, 75, 80, 85, 90, 95% or up to 100% reducing effect on a measured parameter at physiologically-acceptable concentrations (e.g. as exemplified herein).

In some embodiments, the compounds of the invention inhibit EP3-mediated cellular function in pancreatic β cells. In various embodiments, the cellular function includes, but is not limited to, β cell apoptosis and/or impaired insulin secretion, synthesis and/or storage by β-cells. In some embodiments, the cellular function or signaling is induced or enhanced in the presence of free fatty acids (FFA), e.g. palmitate. In a particular embodiment, said compounds inhibit palmitate-induced apoptosis in human islet β cells.

As used herein, an EP3-mediated function or activity means that said function or activity depends on the involvement of a functional EP3 receptor. For example, EP3-mediated functions and activities are inhibited in the presence of specific EP3 antagonists such as (2E)-N-[(5-bromo-2-methoxyphenyl)sulfonyl]-3-[2-(2-naphthalenylmethyl)phenyl]-2-propenamide (L-798106). As disclosed herein, EP3 antagonists according to embodiments of the invention exhibit improved efficacy (e.g. with respect to a function or activity as disclosed herein) as compared to L-798106. For example, the efficacy may be improved by at least 5% and typically by 5-50%. 10-30%, 5-30% or 10-50% as compared to L-798106 at an equivalent physiologically-acceptable concentration.

As disclosed and exemplified herein, β cells (e.g. primary human β cells and murine β cell lines) undergo enhanced programmed cell death (apoptosis) in the presence of fatty acids such as palmitate in an EP3-dependent (mediated) manner. Without wishing to be bound by a specific theory or mechanism of action, fatty acid-induced β cell apoptosis differs from cytokine-induced apoptosis at least in that endoplasmic reticulum (ER) stress appears to be involved in fatty acid-induced apoptosis. Accordingly, a compound that inhibits palmitate-induced apoptosis in human islet β cells is a compound capable of significantly reducing the incidence or degree of β cell apoptosis mediated by palmitate. Beta cell apoptosis may be evaluated by various assays known in the art, including, but not limited to those employing labeled caspase substrates (e.g. caspase 3/7 substrates) and those based on detection of phosphatidylserine or DNA fragmentation. Non-limiting examples of such assays are described throughout the Examples section below. For example, without limitation, compounds in accordance with the invention are exemplified herein to inhibit at least 30% and up to 90% palmitate-induced apoptosis at physiologically-acceptable concentrations of 250 nM-10 μM. In particular, compounds of the invention were exemplified to inhibit at least 70% and typically about 90% palmitate-induced apoptosis in human islet cells, and at least 30-50% palmitate-induced apoptosis in murine β cells.

As further disclosed and exemplified herein, human and murine β cells further exhibit downregulation of insulin gene expression, decreased insulin content in insulin storage vesicles (that may reflect impaired insulin synthesis and/or storage), and impaired insulin secretion (manifested as reduced glucose-stimulated insulin secretion, GSIS), in the presence of fatty acids such as palmitate, in an EP3-dependent manner. The presence and amount of cellular or secreted insulin may be evaluated by various assays known in the art, including, but not limited to, immunoassays such as enzyme-linked immunosorbent assay (ELISA) directed to e.g. human or murine insulin, or to an insulin substrate such as C-peptide (insulin connecting peptide). The level of the insulin transcript may be evaluated or quantified by methods including, but not limited to, RT-PCT, real-time PCR and the like. Non-limitative examples of such assays and methods are described e.g. in Examples 5-6 below.

For example, without limitation, compounds in accordance with the invention are exemplified herein to inhibit palmitate-induced impairment of GSIS in human islets, to thereby substantially restore the GSIS ability of said islet cells (at least 90% and typically 100% inhibition). As further exemplified herein, compounds of the invention restore at least 50% and up to 100% of insulin synthesis in human islets (at least 50% and typically 60-100% inhibition of palmitate-induced impairment).

As used herein, the term “free fatty acids” (FFA) refers to fatty acids that are not part of other molecules, such as triglycerides or phospholipids. FFA encompasses in particular non-glycerol-esterified aliphatic monocarboxylic acids. Palmitate, stearate and oleate are the most abundant FFA, accounting for 70-80% of total plasma FFA. Certain FFA in blood plasma may be non-covalently bound to or adsorbed onto albumin. In experimental assays, palmitic acid (palmitate, either free or in the form of a salt thereof) may be supplemented to the culture media, typically, following complexing with suitable carriers such as bovine serum albumin (BSA) prior to supplementation. By means of a non-limitative example, sodium palmitate at a concentration of 0.1-0.5 mM, typically 0.3 mM, complexed with BSA e.g. at a 1:6 (palmitate to BSA) ratio may conveniently be used to examine palmitate-induced EP3-mediated functions in β cells.

In various embodiments, EP3 antagonists as disclosed herein are identified as antagonists of a mammalian EP3 receptor. In particular, said compounds exhibit inhibition of murine and/or human EP3 signaling or activity as disclosed herein, wherein each possibility represents a separate embodiment of the invention. In some embodiments, the compounds of the invention inhibit signaling or activity mediated by the gamma isoform of EP3 in pancreatic β cells (e.g. in murine β cells). In some embodiments, the compounds of the invention inhibit signaling or activity mediated by EP3-2 in pancreatic β cells (e.g. human). In some embodiments, the compounds of the invention are selective antagonists of EP3. Accordingly, the compounds of the invention typically and advantageously do not inhibit the activity or expression of other non-related receptors or enzymes such as fatty acid synthase (FAS). In some embodiments, compounds of the invention may be selective antagonists of EP3 gamma (and/or its human homolog EP3-2), and do not substantially inhibit other EP3 isoforms at physiologically-acceptable concentrations. In another embodiment, the ability of the compounds of the invention to inhibit EP3 (or an isoform thereof as disclosed herein) is at least 10-fold, or in other embodiments, at least 100-, 1000- or 5,000-10,000-fold greater than its ability to inhibit the activity of other prostaglandin receptors (such as EP1 and EP4). In another embodiment, the affinity of the compounds of the invention to EP3 (or an isoform thereof as disclosed herein) is at least 2, 3, 4, 5, or 10-fold, or in other embodiments, at least 100-, 1000- or 5,000-10,000-fold greater than its affinity to other prostaglandin receptors (such as EP1 and EP4).

### Compounds

In various embodiments, the compounds of the invention are represented by a formula selected from the general formulae I, II and III, as detailed hereinbelow. In other embodiments, the invention employs the use of specific compounds as further detailed below. Certain exemplary compounds of the invention are represented by any one of compounds 1a, 1b, 1c, 2a, 2b, 2c and 3, as detailed hereinbelow, and esters and salts thereof.

According to some embodiments, the compound is represented by Formula I, or a salt thereof, as presented hereinabove. As further detailed herein W of Formula I is —NHSO—, and is positioned between two aromatic rings (i.e., two substituted benzene rings). It is to be understood to the person having ordinary skill in the art that this is intended to mean that either one of the aromatic rings is chemically bonded to either the —NHSOₙ— nitrogen atom or to its sulfur atom. For example, Formulae I-1, Ia and Ib are directed to the option that the aromatic ring bonded to R³ is bonded to the —NHSOₙ— sulfur atom, and the aromatic ring bonded to R¹ and R² is bonded to the —NHSOₙ— nitrogen atom. Also, Formula Ic is directed to the option that the aromatic ring bonded to R³ is bonded to the —NHSOₙ— nitrogen atom, and the aromatic ring bonded to R¹ and R² is bonded to the —NHSOₙ— sulfur atom.

According to some embodiments, the aromatic ring bonded to R³ is bonded to the —NHSOₙ— sulfur atom, and the aromatic ring bonded to R¹ and R² is bonded to the —NHSOₙ— nitrogen atom. According to some embodiments, the aromatic ring bonded to R³ is bonded to the —NHSOₙ-nitrogen atom, and the aromatic ring bonded to R¹ and R² is bonded to the —NHSOₙ— sulfur atom.

According to some embodiments, the compound is represented by Formula I, or a salt thereof, as presented hereinabove, wherein W is —NHSOₙ—, wherein n is 1 or 2; one of R¹ and R² is CONR⁴R⁵, and the other one of R¹ and R² is hydrogen, wherein each one of R⁴ and R⁵ is independently selected from the group consisting of H, alkyl, aryl, arylalkyl, cycloalkyl and haloalkyl, or wherein R⁴ and R⁵ together with the nitrogen atom to which they are bound form a ring, wherein said ring is unsubstituted or substituted with one or more alkyl, haloalkyl and/or halogen; each one of X¹ and Y¹ is independently selected from the group consisting of a halogen and an alkyl; each one of m and k is 0, 1 or 2; and R³ is selected from hydrogen and an alkyl group. According to some embodiments, R³ is selected from hydrogen, methyl and sec-butyl.

According to some embodiments, n is 2. It is apparent from the presentation of Formula I that when n is 2, a sulfonamide group forms as W, wherein the sulfonamide nitrogen atom is covalently bonded to the sulfur atom, to one of the benzene rings of Formula I, and to a hydrogen atom. It is to be understood by a person skilled in the art that such sulfonamides having a N—H bond are mildly acidic, i.e. they are having a tendency to form a relatively stable —SO₂N⁻— anion, while releasing a proton, depending on the ambient pH. According to some embodiments, the compounds of formula I may be provided in a form of a basic salt. Consequently, the phrase “a compound represented by Formula I, or a salt thereof” refers mainly to basic salts of the sulfonamide moiety, according to some embodiments. Other types of salts may be associated with one of the substituents of Formula I, such as R¹, R², R³, X¹ or Y¹. According to some embodiments, the compound of Formula I is provided in the form of a salt.

According to some embodiments, the NHSOₙ nitrogen atom is covalently bonded to the aromatic ring connected to R¹ and R², and the NHSOₙ sulfur atom is covalently bonded to the aromatic ring connected to R³.

According to some embodiments, R² is CONR⁴R⁵, and R¹ is hydrogen.

According to some embodiments, R⁴ and R⁵ together with the nitrogen atom to which they are bound form a ring, which is unsubstituted or substituted with at least one of alkyl, haloalkyl and/or halogen. Specifically, the heterocyclic ring formed from NR⁴R⁵ is devoid of aromatic substituents, such as phenyl and substituted phenyl rings, according to some embodiments.

According to some embodiments, R⁴ and R⁵ together with the nitrogen atom to which they are bound form a ring, wherein the formed ring is unsubstituted or substituted with at least one alkyl, or haloalkyl. According to some embodiments, the formed ring is unsubstituted or substituted with at least one alkyl group. According to some embodiments, the formed ring is substituted with at least one alkyl group. According to some embodiments, the formed ring is substituted with at least two alkyl groups. According to some embodiments, the formed ring is substituted with two alkyl groups. According to some embodiments, each alkyl group, which serves as substituent to the ring formed from NR⁴R⁵ is a C₁-C₆ unsubstituted alkyl. According to some embodiments, each alkyl group, which serves as substituent to the ring formed from NR⁴R⁵ is a C₁-C₆ linear alkyl. According to some embodiments, each alkyl group, which serves as substituent to the ring formed from NR⁴R⁵ is a C₁-C₄ unsubstituted alkyl. According to some embodiments, each alkyl group, which serves as substituent to the ring formed from NR⁴R⁵ is a C₁-C₄ linear alkyl. According to some embodiments, each alkyl group, which serves as substituent to the ring formed from NR⁴R⁵ is a C₁-C₂ unsubstituted alkyl. According to some embodiments, each alkyl group, which serves as substituent to the ring formed from NR⁴R⁵ is selected from ethyl and methyl. According to some embodiments, each alkyl group, which serves as substituent to the ring formed from NR⁴R⁵ is a C₁-C₂ linear alkyl. According to some embodiments, R⁴ and R⁵ together with the nitrogen atom to which they are bound form a ring, which is substituted with two alkyl groups, wherein each alkyl group is selected from methyl and ethyl.

According to some embodiments, the ring formed from NR⁴R⁵ is an N-heterocycle selected from pyrrolidine, pyrrole, oxazole, succinimide, piperidine, pyridine, pyrimidine, piperazine, morpholine, thiomorpholine, indoline and indole, wherein each N-heterocycle is optionally substituted with one or more substituents selected from the group consisting of alkyl, haloalkyl, and halogen. Each possibility represents a separate embodiment. In particular, the N-heterocycle may be devoid of aromatic substituents, such as phenyl and its derivatives, according to some embodiments. According to some embodiments, the ring formed from NR⁴R⁵ is an N-heterocycle selected from pyrrolidine, pyrrole, oxazole, succinimide, pyridine, pyrimidine, piperazine, morpholine, thiomorpholine, indoline and indole, wherein each N-heterocycle is optionally substituted with one or more substituents selected from the group consisting of alkyl, haloalkyl, and halogen. According to some embodiments, the ring formed from NR⁴R⁵ is an N-heterocycle selected from pyrrolidine, and morpholine, wherein each N-heterocycle is optionally substituted with one or more substituents selected from the group consisting of alkyl, haloalkyl, and halogen. According to some embodiments, the N-heterocycle is substituted with one or more of the substituents as described herein. According to some embodiments, the N-heterocycle is substituted with two or more of the substituents. According to some embodiments, the N-heterocycle is substituted with two of the substituents. According to some embodiments, the substituent is an alkyl. According to some embodiments, the substituent is selected from methyl and ethyl.

According to some embodiments, R⁴ and R⁵ together with the nitrogen atom to which they are bound form a ring, wherein the ring is a non-aromatic N-heterocycle. According to some embodiments, the non-aromatic heterocycle is selected from the group consisting of pyrrolidine, succinimide, piperidine, morpholine, thiomorpholine and indoline, wherein each non-aromatic N-heterocycle is optionally substituted with one or more substituents selected from the group consisting of alkyl, haloalkyl, and halogen. Each possibility represents a separate embodiment. In particular, the non-aromatic N-heterocycle may be devoid of aromatic substituents, such as phenyl and its derivatives, according to some embodiments. According to some embodiments, the non-aromatic heterocycle is selected from the group consisting of pyrrolidine, succinimide, morpholine, thiomorpholine and indoline, wherein each non-aromatic N-heterocycle is optionally substituted with one or more substituents selected from the group consisting of alkyl, haloalkyl, and halogen. According to some embodiments, the ring formed from NR⁴R⁵ is a non-aromatic N-heterocycle selected from pyrrolidine, and morpholine, wherein each non-aromatic N-heterocycle is optionally substituted with one or more substituents selected from the group consisting of alkyl, haloalkyl, and halogen. According to some embodiments, the non-aromatic N-heterocycle is substituted with one or more of the substituents. According to some embodiments, the N-heterocycle is substituted with two or more of the substituents. According to some embodiments, the non-aromatic N-heterocycle is substituted with two of the substituents. According to some embodiments, the substituent is an alkyl. According to some embodiments, the substituent is selected from methyl and ethyl.

According to some embodiments, N-heterocycle is pyrrolidine. According to some embodiments, the non-aromatic N-heterocycle is pyrrolidine. According to some embodiments, R⁴ and R⁵ together with the nitrogen atom to which they are bound form a ring, wherein the ring is a pyrrolidine, which is optionally substituted with one or more substituents selected from the group consisting of alkyl, haloalkyl, and halogen. Each possibility represents a separate embodiment. According to some embodiments, the pyrrolidine is substituted with one or more of the substituents. According to some embodiments, the pyrrolidine is substituted with two or more of the substituents. According to some embodiments, the pyrrolidine is substituted with two of the substituents. According to some embodiments, the substituent is an alkyl. According to some embodiments, the pyrrolidine is substituted with two alkyl groups. According to some embodiments, each of the alkyl groups is a C₁-C₆ alkyl. According to some embodiments, each of the alkyl groups is a C₁-C₆ unsubstituted alkyl. According to some embodiments, each of the alkyl groups is a C₁-C₆ linear alkyl. According to some embodiments, each of the alkyl groups is a C₁-C₄ alkyl. According to some embodiments, each of the alkyl groups is a C₁-C₄ unsubstituted alkyl. According to some embodiments, each of the alkyl groups is a C₁-C₄ linear alkyl.

According to some embodiments, each of the alkyl groups is selected from the group consisting of methyl, ethyl, n-propyl, isopropyl, n-butyl and tert-butyl. Each possibility represents a separate embodiment. According to some embodiments, each of the alkyl groups is selected from the group consisting of methyl and ethyl.

According to some embodiments, R⁴ and R⁵ together with the nitrogen atom to which they are bound form the ring:

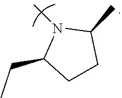

Thus, according to some embodiments, R² is:

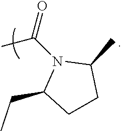

According to some embodiments, Y¹ is selected from the group consisting of a halogen and an alkyl. According to some embodiments, when m is 2, each one of Y¹ is independently selected from the group consisting of a halogen and an alkyl.

According to some embodiments, m is 0 or 1. According to some embodiments, m is 0. It is to be understood that when m is 0, the benzene ring, to which R¹ and R² are attached, is covalently bonded to four hydrogen atoms, to one of R¹ and R² and to the aromatic nitrogen atom.

According to some embodiments, X¹ is selected from the group consisting of a halogen and an alkyl. According to some embodiments, when k is 2, each one of X¹ is independently selected from the group consisting of a halogen and an alkyl. According to some embodiments, X¹ is a halogen. According to some embodiments, when k is 2, each one of X¹ is independently a halogen.

According to some embodiments, k is 0 or 1. According to some embodiments, k is 1. According to some embodiments, k is 1 and X¹ is a halogen. According to some embodiments, X¹ is selected from the group consisting of fluorine, chlorine and bromine. Each possibility represents a separate embodiment. According to some embodiments, X¹ is selected from the group consisting of fluorine and chlorine. According to some embodiments, X¹ is fluorine.

According to some embodiments k is 1 and X¹ is positioned ortho to R³. According to some embodiments, k is 1. According to some embodiments, k is 1 and X¹ is a halogen positioned ortho to R³. According to some embodiments, k is 1. According to some embodiments, k is 1 and X¹ is a fluorine atom positioned ortho to R³.

According to some embodiments, R³ is a C₁-C₆ alkyl. According to some embodiments, R³ is a C₁-C₄ alkyl. According to some embodiments, R³ is an unsubstituted alkyl. According to some embodiments, R³ is a linear alkyl. According to some embodiments, R³ is a branched alkyl.

According to some embodiments, R³ is selected from the group consisting of methyl, ethyl, n-propyl, isopropyl, n-butyl, sec-butyl and tert-butyl. Each possibility represents a separate embodiment. According to some embodiments, R³ is selected from the group consisting of methyl and sec-butyl. According to some embodiments, R³ is methyl. According to some embodiments, R³ is methyl.

According to some embodiments, the compound is Compound 1a, as presented herein above, or a salt thereof.

According to some embodiments, the NHSOₙ nitrogen atom is covalently bonded to the aromatic ring connected to R¹ and R², and the NHSOₙ sulfur atom is covalently bonded to the aromatic ring connected to R³.

According to some embodiments, R¹ is CONR⁴R⁵, and R² is hydrogen.

According to some embodiments, R⁴ and R⁵ together with the nitrogen atom to which they are bound form a ring, which is unsubstituted or substituted with at least one of alkyl, haloalkyl and/or halogen. Specifically, the heterocyclic ring formed from NR⁴R⁵ is devoid of aromatic substituents, such as phenyl and substituted phenyl rings, according to some embodiments.

According to some embodiments, R⁴ and R⁵ together with the nitrogen atom to which they are bound form a ring, wherein the formed ring is unsubstituted or substituted with at least one alkyl, or haloalkyl. According to some embodiments, the formed ring is unsubstituted or substituted with at least one alkyl group. According to some embodiments, the formed ring is substituted with at least one alkyl group. According to some embodiments, the formed ring is substituted with at least two alkyl groups. According to some embodiments, the formed ring is substituted with two alkyl groups. According to some embodiments, each alkyl group, which serves as substituent to the ring formed from NR⁴R⁵ is a C₁-C₆ unsubstituted alkyl. According to some embodiments, each alkyl group, which serves as substituent to the ring formed from NR⁴R⁵ is a C₁-C₆ linear alkyl. According to some embodiments, each alkyl group, which serves as substituent to the ring formed from NR⁴R⁵ is a C₁-C₄ unsubstituted alkyl. According to some embodiments, each alkyl group, which serves as substituent to the ring formed from NR⁴R⁵ is a C₁-C₄ linear alkyl. According to some embodiments, each alkyl group, which serves as substituent to the ring formed from NR⁴R⁵ is a C₁-C₂ unsubstituted alkyl. According to some embodiments, each alkyl group, which serves as substituent to the ring formed from NR⁴R⁵ is selected from ethyl and methyl. According to some embodiments, each alkyl group, which serves as substituent to the ring formed from NR⁴R⁵ is a C₁-C₂ linear alkyl. According to some embodiments, R⁴ and R⁵ together with the nitrogen atom to which they are bound form a ring, which is substituted with two alkyl groups, wherein each alkyl group is selected from methyl and ethyl.

According to some embodiments, the ring formed from NR⁴R⁵ is an N-heterocycle selected from the group consisting of pyrrolidine, pyrrole, oxazole, succinimide, piperidine, pyridine, pyrimidine, piperazine, morpholine, thiomorpholine, indoline and indole, wherein each N-heterocycle is optionally substituted with one or more substituents selected from the group consisting of alkyl, haloalkyl, and halogen. Each possibility represents a separate embodiment. In particular, the N-heterocycle may be devoid of aromatic substituents, such as phenyl and its derivatives, according to some embodiments. According to some embodiments, the ring formed from NR⁴R⁵ is an N-heterocycle selected from pyrrolidine, pyrrole, oxazole, succinimide, pyridine, pyrimidine, piperazine, morpholine, thiomorpholine, indoline and indole, wherein each N-heterocycle is optionally substituted with one or more substituents selected from the group consisting of alkyl, haloalkyl, and halogen. According to some embodiments, the ring formed from NR⁴R⁵ is an N-heterocycle selected from pyrrolidine, and morpholine, wherein each N-heterocycle is optionally substituted with one or more substituents selected from the group consisting of alkyl, haloalkyl, and halogen. According to some embodiments, the N-heterocycle is substituted with one or more of the substituents. According to some embodiments, the N-heterocycle is substituted with two or more of the substituents. According to some embodiments, the N-heterocycle is substituted with two of the substituents. According to some embodiments, the substituent is an alkyl. According to some embodiments, the substituent is selected from methyl and ethyl.

According to some embodiments, R⁴ and R⁵ together with the nitrogen atom to which they are bound form a ring, wherein the ring is a non-aromatic N-heterocycle. According to some embodiments, the non-aromatic heterocycle is selected from the group consisting of pyrrolidine, succinimide, piperidine, morpholine, thiomorpholine and indoline, wherein each non-aromatic N-heterocycle is optionally substituted with one or more substituents selected from the group consisting of alkyl, haloalkyl, and halogen. Each possibility represents a separate embodiment. In particular, the non-aromatic N-heterocycle may be devoid of aromatic substituents, such as phenyl and its derivatives, according to some embodiments. According to some embodiments, the non-aromatic heterocycle is selected from the group consisting of pyrrolidine, succinimide, morpholine, thiomorpholine and indoline, wherein each non-aromatic N-heterocycle is optionally substituted with one or more substituents selected from the group consisting of alkyl, haloalkyl, and halogen. According to some embodiments, the ring formed from NR⁴R⁵ is a non-aromatic N-heterocycle selected from pyrrolidine, and morpholine, wherein each non-aromatic N-heterocycle is optionally substituted with one or more substituents selected from the group consisting of alkyl, haloalkyl, and halogen.

According to some embodiments, the non-aromatic N-heterocycle is substituted with one or more of the substituents. According to some embodiments, the N-heterocycle is substituted with two or more of the substituents. According to some embodiments, the non-aromatic N-heterocycle is substituted with two of the substituents. According to some embodiments, the substituent is an alkyl. According to some embodiments, N-heterocycle is morpholine. According to some embodiments, the non-aromatic N-heterocycle is morpholine. According to some embodiments, R⁴ and R⁵ together with the nitrogen atom to which they are bound form a ring, wherein the ring is a morpholine, which is optionally substituted with one or more substituents selected from the group consisting of alkyl, haloalkyl, and halogen. Each possibility represents a separate embodiment. According to some embodiments, the morpholine is substituted with one or more of the substituents. According to some embodiments, the morpholine is substituted with two or more of the substituents. According to some embodiments, the morpholine is substituted with two of the substituents. According to some embodiments, the substituent is an alkyl. According to some embodiments, the pyrrolidine is substituted with two alkyl groups. According to some embodiments, each of the alkyl groups is a C₁-C₆ alkyl. According to some embodiments, each of the alkyl groups is a C₁-C₆ unsubstituted alkyl. According to some embodiments, each of the alkyl groups is a C₁-C₆ linear alkyl. According to some embodiments, each of the alkyl groups is a C₁-C₄ alkyl. According to some embodiments, each of the alkyl groups is a C₁-C₄ unsubstituted alkyl. According to some embodiments, each of the alkyl groups is a C₁-C₄ linear alkyl.

According to some embodiments, each of the alkyl groups is selected from the group consisting of methyl, ethyl, n-propyl, isopropyl, n-butyl and tert-butyl. Each possibility represents a separate embodiment. According to some embodiments, each of the alkyl groups is selected from the group consisting of methyl and ethyl.

According to some embodiments, R⁴ and R⁵ together with the nitrogen atom to which they are bound form the ring:

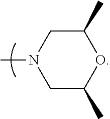

According to some embodiments, R¹ is:

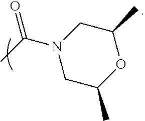

According to some embodiments, Y¹ is selected from the group consisting of a halogen and an alkyl. According to some embodiments, when m is 2, each one of Y¹ is independently selected from the group consisting of a halogen and an alkyl.

According to some embodiments, m is 0 or 1. According to some embodiments, m is 0.

According to some embodiments, X¹ is selected from the group consisting of a halogen and an alkyl. According to some embodiments, when k is 2, each one of X¹ is independently selected from the group consisting of a halogen and an alkyl. According to some embodiments, X¹ is a halogen. According to some embodiments, when k is 2, each one of X¹ is independently a halogen.

According to some embodiments, k is 0 or 1. According to some embodiments, k is 0.

According to some embodiments, R³ is a C₁-C₆ alkyl. According to some embodiments, R³ is a C₁-C₄ alkyl. According to some embodiments, R³ is an unsubstituted alkyl. According to some embodiments, R³ is a linear alkyl. According to some embodiments, R³ is a branched alkyl.

According to some embodiments, R³ is selected from the group consisting of methyl, ethyl, n-propyl, isopropyl, n-butyl, sec-butyl and tert-butyl. Each possibility represents a separate embodiment. According to some embodiments, R³ is selected from the group consisting of methyl and sec-butyl. According to some embodiments, R³ is methyl. According to some embodiments, R³ is sec-butyl According to some embodiments, the compound is Compound 1b as presented herein above.

According to some embodiments, the NHSOₙ sulfur atom is covalently bonded to the aromatic ring connected to R¹ and R², and the NHSOₙ nitrogen atom is covalently bonded to the aromatic ring connected to R³.

According to some embodiments, R² is CONR⁴R⁵, and R¹ is hydrogen.

According to some embodiments, R⁴ and R⁵ together with the nitrogen atom to which they are bound form a ring wherein the formed ring is unsubstituted or substituted with at least one alkyl, halogen, or haloalkyl. Specifically, the heterocyclic ring formed from NR⁴R⁵ is devoid of aromatic substituents, such as phenyl and substituted phenyl rings, according to some embodiments.

According to some embodiments, R⁴ and R⁵ together with the nitrogen atom to which they are bound form a ring, wherein the formed ring is unsubstituted or substituted with at least one alkyl, or haloalkyl. According to some embodiments, the formed ring is unsubstituted or substituted with at least one alkyl group. According to some embodiments, the formed ring is substituted with at least one alkyl group. According to some embodiments, the formed ring is substituted with at least two alkyl groups. According to some embodiments, the formed ring is substituted with two alkyl groups. According to some embodiments, each alkyl group, which serves as substituent to the ring formed from NR⁴R⁵ is a C₁-C₆ unsubstituted alkyl. According to some embodiments, each alkyl group, which serves as substituent to the ring formed from NR⁴R⁵ is a C₁-C₆ linear alkyl. According to some embodiments, each alkyl group, which serves as substituent to the ring formed from NR⁴R⁵ is a C₁-C₄ unsubstituted alkyl. According to some embodiments, each alkyl group, which serves as substituent to the ring formed from NR⁴R⁵ is a C₁-C₄ linear alkyl. According to some embodiments, each alkyl group, which serves as substituent to the ring formed from NR⁴R⁵ is a C₁-C₂ unsubstituted alkyl. According to some embodiments, each alkyl group, which serves as substituent to the ring formed from NR⁴R⁵ is selected from ethyl and methyl. According to some embodiments, each alkyl group, which serves as substituent to the ring formed from NR⁴R⁵ is a C₁-C₂ linear alkyl. According to some embodiments, R⁴ and R⁵ together with the nitrogen atom to which they are bound form a ring, which is substituted with two alkyl groups, wherein each alkyl group is selected from methyl and ethyl.

According to some embodiments, the ring formed from NR⁴R⁵ is an N-heterocycle selected from the group consisting of pyrrolidine, pyrrole, oxazole, succinimide, piperidine, pyridine, pyrimidine, piperazine, morpholine, thiomorpholine, indoline and indole, wherein each N-heterocycle is optionally substituted with one or more substituents selected from the group consisting of alkyl, haloalkyl, and halogen. Each possibility represents a separate embodiment. In particular, the N-heterocycle may be devoid of aromatic substituents, such as phenyl and its derivatives, according to some embodiments. According to some embodiments, the ring formed from NR⁴R⁵ is an N-heterocycle selected from pyrrolidine, pyrrole, oxazole, succinimide, pyridine, pyrimidine, piperazine, morpholine, thiomorpholine, indoline and indole, wherein each N-heterocycle is optionally substituted with one or more substituents selected from the group consisting of alkyl, haloalkyl, and halogen. According to some embodiments, the ring formed from NR⁴R⁵ is an N-heterocycle selected from pyrrolidine, and morpholine, wherein each N-heterocycle is optionally substituted with one or more substituents selected from the group consisting of alkyl, haloalkyl, and halogen.

According to some embodiments, the N-heterocycle is substituted with one or more of the substituents as described herein. According to some embodiments, the N-heterocycle is substituted with two or more of the substituents. According to some embodiments, the N-heterocycle is substituted with two of the substituents. According to some embodiments, the substituent is an alkyl. According to some embodiments, the substituent is selected from methyl and ethyl.

According to some embodiments, R⁴ and R⁵ together with the nitrogen atom to which they are bound form a ring, wherein the ring is a non-aromatic N-heterocycle. According to some embodiments, the non-aromatic heterocycle is selected from the group consisting of pyrrolidine, succinimide, piperidine, morpholine, thiomorpholine and indoline, wherein each non-aromatic N-heterocycle is optionally substituted with one or more substituents selected from the group consisting of alkyl, haloalkyl, and halogen. Each possibility represents a separate embodiment. In particular, the non-aromatic N-heterocycle may be devoid of aromatic substituents, such as phenyl and its derivatives, according to some embodiments. According to some embodiments, the non-aromatic heterocycle is selected from the group consisting of pyrrolidine, succinimide, morpholine, thiomorpholine and indoline, wherein each non-aromatic N-heterocycle is optionally substituted with one or more substituents selected from the group consisting of alkyl, haloalkyl, and halogen. According to some embodiments, the ring formed from NR⁴R⁵ is a non-aromatic N-heterocycle selected from pyrrolidine, and morpholine, wherein each non-aromatic N-heterocycle is optionally substituted with one or more substituents selected from the group consisting of alkyl, haloalkyl, and halogen.

According to some embodiments, the non-aromatic N-heterocycle is substituted with one or more of the substituents. According to some embodiments, the N-heterocycle is substituted with two or more of the substituents. According to some embodiments, the non-aromatic N-heterocycle is substituted with two of the substituents. According to some embodiments, the substituent is an alkyl. According to some embodiments, N-heterocycle is morpholine. According to some embodiments, the non-aromatic N-heterocycle is morpholine. According to some embodiments, R⁴ and R⁵ together with the nitrogen atom to which they are bound form a ring, wherein the ring is a morpholine, which is optionally substituted with one or more substituents selected from the group consisting of alkyl, haloalkyl, and halogen. Each possibility represents a separate embodiment.

According to some embodiments, the morpholine is substituted with one or more of the substituents. According to some embodiments, the morpholine is substituted with two or more of the substituents. According to some embodiments, the morpholine is substituted with two of the substituents. According to some embodiments, the substituent is an alkyl. According to some embodiments, the pyrrolidine is substituted with two alkyl groups. According to some embodiments, each of the alkyl groups is a C₁-C₆ alkyl. According to some embodiments, each of the alkyl groups is a C₁-C₆ unsubstituted alkyl. According to some embodiments, each of the alkyl groups is a C₁-C₆ linear alkyl. According to some embodiments, each of the alkyl groups is a C₁-C₄ alkyl. According to some embodiments, each of the alkyl groups is a C₁-C₄ unsubstituted alkyl. According to some embodiments, each of the alkyl groups is a C₁-C₄ linear alkyl.

According to some embodiments, each of the alkyl groups is selected from the group consisting of methyl, ethyl, n-propyl, isopropyl, n-butyl and tert-butyl. Each possibility represents a separate embodiment.

According to some embodiments, R⁴ and R⁵ together with the nitrogen atom to which they are bound form the ring:

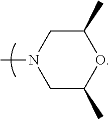

According to some embodiments, R² is:

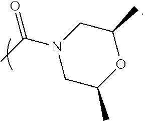

According to some embodiments, Y¹ is selected from the group consisting of a halogen and an alkyl. According to some embodiments, when m is 2, each one of Y¹ is independently selected from the group consisting of a halogen and an alkyl.

According to some embodiments, m is 1 or 2. According to some embodiments, m is 2. According to some embodiments, m is 2, and each one of Y¹ is independently a halogen. According to some embodiments, the halogen is chlorine. According to some embodiments, one of the chlorine atoms is positioned ortho to R² and the other chlorine atom is positioned ortho to W. According to some embodiments, one of the chlorine atoms is positioned ortho to R² and the other chlorine atom is positioned para to R².

According to some embodiments, X¹ is selected from the group consisting of a halogen and an alkyl. According to some embodiments, when k is 2, each one of X¹ is independently selected from the group consisting of a halogen and an alkyl. According to some embodiments, X¹ is a halogen. According to some embodiments, when k is 2, each one of X¹ is independently a halogen.

According to some embodiments, k is 0 or 1. According to some embodiments, k is 1.

According to some embodiments, k is 1 and X¹ is a halogen. According to some embodiments, X¹ is selected from the group consisting of fluorine, chlorine and bromine. Each possibility represents a separate embodiment. According to some embodiments, X¹ is selected from the group consisting of fluorine and chlorine. According to some embodiments, X¹ is fluorine.

According to some embodiments k is 1 and X¹ is positioned ortho to R³. According to some embodiments, k is 1. According to some embodiments, k is 1 and X¹ is a halogen positioned ortho to R³. According to some embodiments, k is 1. According to some embodiments, k is 1 and X¹ is a fluorine atom positioned ortho to R³.

According to some embodiments, R³ is H.

According to some embodiments, the compound of Formula I is Compound 1c, as presented herein above or a salt thereof:

According to some embodiments, the compound is selected from Compound 1a, Compound 1b, Compound 1c, as presented hereinabove and salts thereof:

According to some embodiments, Compound 1a is also referred to herein as compound C13. According to some embodiments, Compound 1a also includes any derivatives or salts thereof. The generic name of Compound 1a is N-(3-((2R,5S)-2-ethyl-5-methylpyrrolidine-1-carbonyl)phenyl)-3-fluoro-4-methylbenzenesulfonamide.

Specifically, according to some embodiments, Compound 1a corresponds to Formula I, wherein the NHSOₙ nitrogen atom is covalently bonded to the aromatic ring connected to R¹ and R², and the NHSOₙ sulfur atom is covalently bonded to the aromatic ring connected to R³; n is 2; R¹ is H; R² is CONR⁴R⁵; R⁴ and R⁵ together with the nitrogen atom to which they are bound form a ring, wherein the ring is a 2-ethyl-3-ethyl pyrrolidine having the following formula:

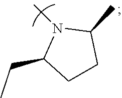

k is 1; X¹ is fluorine positioned ortho to R³; m is 0, such that Y¹ is absent; and R³ is methyl.

According to some embodiments, compound 1b is also referred to herein as compound C5.

According to some embodiments, compound 1b also includes any derivatives or salts thereof.

Specifically, according to some embodiments, Compound 1b corresponds to Formula I, wherein the NHSOₙ nitrogen atom is covalently bonded to the aromatic ring connected to R¹ and R², and the NHSOₙ sulfur atom is covalently bonded to the aromatic ring connected to R³; n is 2; R² is H; R¹ is CONR⁴R⁵; R⁴ and R⁵ together with the nitrogen atom to which they are bound form a ring, wherein the ring is a 2,6-dimethylmorpholine having the following formula:

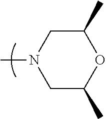

k is 0, such that X¹ is absent; m is 0, such that Y¹ is absent; and R³ is sec-butyl.

According to some embodiments, compound 1b is also referred to herein as compound C5.

According to some embodiments, compound 1b also includes any derivatives or salts thereof. The generic name of Compound 1b is 4-((S)-sec-butyl)-N-(2-((2S,6R)-2,6-dimethylmorpholine-4-carbonyl)phenyl)benzenesulfonamide.

Specifically, according to some embodiments, Compound 1c corresponds to Formula I, wherein in W the NHSOₙ sulfur atom is covalently bonded to the aromatic ring connected to R¹ and R², and the NHSOₙ nitrogen atom is covalently bonded to the aromatic ring connected to R³; n is 2; R¹ is H; R² is CONR⁴R⁵; R⁴ and R⁵ together with the nitrogen atom to which they are bound form a ring, wherein the ring is a 2,6-dimethylmorpholine having the following formula:

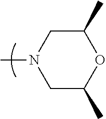

k is 1; X¹ is fluorine positioned ortho to W; m is 2; one of the two Y¹ is chlorine positioned para to R² and the other of the two Y¹ is chlorine positioned para to W; and R³ is H.

According to some embodiments, compound 1c is also referred to herein as compound C14. According to some embodiments, compound 1c also includes any derivatives or salts thereof. The generic name of Compound 1c is 2,4-dichloro-5-((2R,6S)-2,6-dimethylmorpholine-4-carbonyl)-N-(2-fluorophenyl)benzenesulfonamide.

According to some embodiments, the compound is selected from Compound 1a, Compound 1b or a salt thereof. According to some embodiments, the compound is selected from Compound 1a, Compound 1c or a salt thereof. According to some embodiments, the compound is selected from Compound 1b, Compound 1c or a salt thereof. According to some embodiments, the compound is Compound 1a or a salt thereof. According to some embodiments, the compound is Compound 1b or a salt thereof. According to some embodiments, the compound is Compound 1c or a salt thereof.

According to some embodiments, the compound is represented by Formula I-1, or a salt thereof:

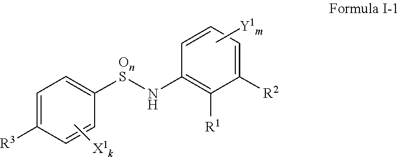

wherein
n is 1 or 2;
one of R¹ and R² is CONR⁴R⁵, and the other one of R¹ and R² is hydrogen, wherein each one of R⁴ and R⁵ is independently selected from the group consisting of H, alkyl, aryl, arylalkyl, cycloalkyl and haloalkyl, or wherein R⁴ and R⁵ together with the nitrogen atom to which they are bound form a ring, wherein the formed ring is unsubstituted or substituted with at least one alkyl, halogen, or haloalkyl;
each one of X¹ and Y¹ is independently selected from the group consisting of a halogen and an alkyl;
each one of m and k is 0, 1 or 2; and
R³ is selected from hydrogen and an alkyl group.

Specifically, Formula I-1 represents an embodiment of Formula I, wherein W is defined such that the NHSOₙ nitrogen atom is covalently bonded to the aromatic ring connected to R¹ and R², and the NHSOₙ sulfur atom is covalently bonded to the aromatic ring connected to R³. Various embodiments relating to the other substituents and numbers of Formula I-1 (e.g. n, k, m, R¹, R², R³, X¹, Y¹) are as presented herein, when referring to Formula I.

According to some embodiments, the compound is selected from Compound 1a, Compound 1b and salts thereof, the formulae thereof is presented herein above.

According to some embodiments, the compound is represented by Formula Ia, or a salt thereof:

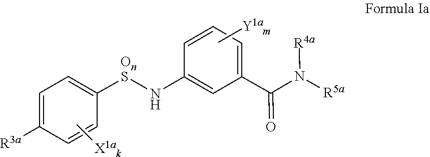

wherein
n is 1 or 2;
each one of R^{4a} and R^{5a} is independently selected from the group consisting of H, alkyl, aryl, arylalkyl, cycloalkyl and haloalkyl, or wherein R^{4a} and R^{5a} together with the nitrogen atom to which they are bound form a ring, wherein the formed ring is unsubstituted or substituted with at least one alkyl, halogen, or haloalkyl;
each one of X^{1a} and Y^{1a} is independently selected from the group consisting of a halogen and an alkyl;
each one of m and k is 0, 1 or 2; and
R^{3a} is selected from hydrogen and an alkyl group.

According to some embodiments, R^{4a} and R^{5a} together with the nitrogen atom to which they are bound form a ring, which is unsubstituted or substituted with at least one of alkyl, haloalkyl and/or halogen. Specifically, the heterocyclic ring formed from NR^{4a}R^{5a} is devoid of aromatic substituents, such as phenyl and substituted phenyl rings, according to some embodiments.

According to some embodiments, R^{4a} and R^{5a} together with the nitrogen atom to which they are bound form a ring, wherein the formed ring is unsubstituted or substituted with at least one alkyl, or haloalkyl. According to some embodiments, the formed ring is unsubstituted or substituted with at least one alkyl group. According to some embodiments, the formed ring is substituted with at least one alkyl group. According to some embodiments, the formed ring is substituted with at least two alkyl groups. According to some embodiments, the formed ring is substituted with two alkyl groups. According to some embodiments, each alkyl group, which serves as substituent to the ring formed from NR^{4a}R^{5a} is a C₁-C₆ unsubstituted alkyl. According to some embodiments, each alkyl group, which serves as substituent to the ring formed from NR^{4a}R^{5a} is a C₁-C₆ linear alkyl. According to some embodiments, each alkyl group, which serves as substituent to the ring formed from NR^{4a}R^{5a} is a C₁-C₄ unsubstituted alkyl. According to some embodiments, each alkyl group, which serves as substituent to the ring formed from NR^{4a}R^{5a} is a C₁-C₄ linear alkyl. According to some embodiments, each alkyl group, which serves as substituent to the ring formed from NR^{4c}R^{5a} is a C₁-C₂ unsubstituted alkyl. According to some embodiments, each alkyl group, which serves as substituent to the ring formed from NR^{4a}R^{5a} is selected from ethyl and methyl. According to some embodiments, each alkyl group, which serves as substituent to the ring formed from NR^{4a}R^{5a} is a C₁-C₂ linear alkyl. According to some embodiments, R^{4a} and R^{5a} together with the nitrogen atom to which they are bound form a ring, which is substituted with two alkyl groups, wherein each alkyl group is selected from methyl and ethyl.

According to some embodiments, the ring formed from NR^{4a}R^{5a} is an N-heterocycle selected from pyrrolidine, pyrrole, oxazole, succinimide, piperidine, pyridine, pyrimidine, piperazine, morpholine, thiomorpholine, indoline and indole, wherein each N-heterocycle is optionally substituted with one or more substituents selected from the group consisting of alkyl, haloalkyl, and halogen. In particular, the N-heterocycle may be devoid of aromatic substituents, such as phenyl and its derivatives, according to some embodiments. According to some embodiments, the ring formed from NR⁴R⁵ is an N-heterocycle selected from pyrrolidine, pyrrole, oxazole, succinimide, pyridine, pyrimidine, piperazine, morpholine, thiomorpholine, indoline and indole, wherein each N-heterocycle is optionally substituted with one or more substituents selected from the group consisting of alkyl, haloalkyl, and halogen. According to some embodiments, the ring formed from NR⁴R⁵ is an N-heterocycle selected from pyrrolidine, and morpholine, wherein each N-heterocycle is optionally substituted with one or more substituents selected from the group consisting of alkyl, haloalkyl, and halogen. According to some embodiments, the N-heterocycle is substituted with one or more of the substituents. According to some embodiments, the N-heterocycle is substituted with two or more of the substituents. According to some embodiments, the N-heterocycle is substituted with two of the substituents. According to some embodiments, the substituent is an alkyl. According to some embodiments, the substituent is selected from methyl and ethyl.

According to some embodiments, R^{4a} and R^{5a} together with the nitrogen atom to which they are bound form a ring, wherein the ring is a non-aromatic N-heterocycle. According to some embodiments, the non-aromatic heterocycle is selected from the group consisting of pyrrolidine, succinimide, piperidine, morpholine, thiomorpholine and indoline, wherein each non-aromatic N-heterocycle is optionally substituted with one or more substituents selected from the group consisting of alkyl, haloalkyl, and halogen. Each possibility represents a separate embodiment. In particular, the non-aromatic N-heterocycle may be devoid of aromatic substituents, such as phenyl and its derivatives, according to some embodiments. According to some embodiments, the non-aromatic heterocycle is selected from the group consisting of pyrrolidine, succinimide, morpholine, thiomorpholine and indoline, wherein each non-aromatic N-heterocycle is optionally substituted with one or more substituents selected from the group consisting of alkyl, haloalkyl, and halogen. According to some embodiments, the ring formed from NR⁴R⁵ is a non-aromatic N-heterocycle selected from pyrrolidine, and morpholine, wherein each non-aromatic N-heterocycle is optionally substituted with one or more substituents selected from the group consisting of alkyl, haloalkyl, and halogen. According to some embodiments, the non-aromatic N-heterocycle is substituted with one or more of the substituents. According to some embodiments, the N-heterocycle is substituted with two or more of the substituents. According to some embodiments, the non-aromatic N-heterocycle is substituted with two of the substituents. According to some embodiments, the substituent is an alkyl. According to some embodiments, the substituent is selected from methyl and ethyl.

According to some embodiments, N-heterocycle is pyrrolidine. According to some embodiments, the non-aromatic N-heterocycle is pyrrolidine. According to some embodiments, R^{4a} and R^{5a} together with the nitrogen atom to which they are bound form a ring, wherein the ring is a pyrrolidine, which is optionally substituted with one or more substituents selected from the group consisting of alkyl, haloalkyl, and halogen. According to some embodiments, the pyrrolidine is substituted with one or more of the substituents. According to some embodiments, the pyrrolidine is substituted with two or more of the substituents. According to some embodiments, the pyrrolidine is substituted with two of the substituents. According to some embodiments, the substituent is an alkyl. According to some embodiments, the pyrrolidine is substituted with two alkyl groups. According to some embodiments, each of the alkyl groups is a C₁-C₆ alkyl. According to some embodiments, each of the alkyl groups is a C₁-C₆ unsubstituted alkyl. According to some embodiments, each of the alkyl groups is a C₁-C₆ linear alkyl.

According to some embodiments, each of the alkyl groups is a C₁-C₄ alkyl. According to some embodiments, each of the alkyl groups is a C₁-C₄ unsubstituted alkyl. According to some embodiments, each of the alkyl groups is a C₁-C₄ linear alkyl. According to some embodiments, each of the alkyl groups is selected from the group consisting of methyl and ethyl.

According to some embodiments, each of the alkyl groups is selected from the group consisting of methyl, ethyl, n-propyl, isopropyl, n-butyl and tert-butyl. Each possibility represents a separate embodiment.

According to some embodiments, R^{4a} and R^{5a} together with the nitrogen atom to which they are bound form the ring:

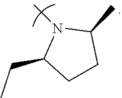

According to some embodiments, Y^{1a} is selected from the group consisting of a halogen and an alkyl. According to some embodiments, when m is 2, each one of Y^{1a} is independently selected from the group consisting of a halogen and an alkyl.

According to some embodiments, m is 0 or 1. According to some embodiments, m is 0. It is to be understood that when m is 0, the benzene ring, to which the CONR^{4a}R^{5a} is attached, is covalently bonded to four hydrogen atoms, to CONR^{4a}R^{5a} and to the aromatic nitrogen atom.

According to some embodiments, n is 2. It is apparent from the presentation of Formula Ia that when n is 2, a sulfonamide group forms, wherein the sulfonamide nitrogen atom is covalently bonded to the sulfur atom, to one of the benzene rings of Formula Ia, and to a hydrogen atom. It is to be understood by a person skilled in the art that such sulfonamides having a N—H bond are mildly acidic, i.e. they are having a tendency to form a relatively stable —SO₂N⁻— anion, while releasing a proton, depending on the ambient pH. According to some embodiments, the compounds of formula Ia may be provided in a form of a basic salt. Consequently, the phrase “a compound represented by Formula Ia, or a salt thereof” refers mainly to basic salts of the sulfonamide moiety, according to some embodiments. Other types of salts may be associated with one of the substituents of Formula Ia, such as R^{3a}, R^{4a}, R^{5a}, X^{1a} or Y^{1a}.

According to some embodiments, the compound of Formula Ia is provided in the form of a salt.

According to some embodiments, X^{1a} is selected from the group consisting of a halogen and an alkyl. According to some embodiments, when k is 2, each one of X^{1a} is independently selected from the group consisting of a halogen and an alkyl. According to some embodiments, X^{1a} is a halogen. According to some embodiments, when k is 2, each one of X¹ is independently a halogen.

According to some embodiments, k is 0 or 1. According to some embodiments, k is 1.

According to some embodiments, k is 1 and X^{1a} is a halogen. According to some embodiments, X^{1a} is selected from the group consisting of fluorine, chlorine and bromine. Each possibility represents a separate embodiment. According to some embodiments, X^{1a} is selected from the group consisting of fluorine and chlorine. According to some embodiments, X^{1a} is fluorine.

According to some embodiments k is 1 and X^{1a} is positioned ortho to R^{3a}. According to some embodiments, k is 1. According to some embodiments, k is 1 and X^{1a} is a halogen positioned ortho to R^{3a}. According to some embodiments, k is 1. According to some embodiments, k is 1 and X^{1a} is a fluorine atom positioned ortho to R^{3a}.

According to some embodiments, R^{3a} is a C₁-C₆ alkyl. According to some embodiments, R^{3a} is a C₁-C₄ alkyl. According to some embodiments, R^{3a} is an unsubstituted alkyl. According to some embodiments, R^{3a} is a linear alkyl. According to some embodiments, R^{3a} is selected from the group consisting of methyl, ethyl, n-propyl, isopropyl, n-butyl, sec-butyl and tert-butyl. Each possibility represents a separate embodiment. According to some embodiments, R^{3a} is selected from the group consisting of methyl and sec-butyl. According to some embodiments, R^{3a} is methyl.

According to some embodiments, the compound of Formula Ia is Compound 1a, as presented herein above or a salt thereof.

According to some embodiments, compound 1a is also referred to herein as compound C13.

According to some embodiments, compound 1a also includes any derivatives or salts thereof.

Specifically, according to some embodiments, Compound 1a corresponds to Formula Ia, wherein n is 2; R^{4a} and R^{5a} together with the nitrogen atom to which they are bound form a ring, wherein the ring is a 2-ethyl-3-ethyl pyrrolidine having the following formula:

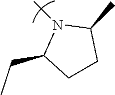

k is 1; X^{1a} is fluorine positioned ortho to R^{3a}; m is 0, such that Y^{1a} is absent; and R^{3a} is methyl. According to some embodiments, the compound is represented by Formula Ib, or a salt thereof:

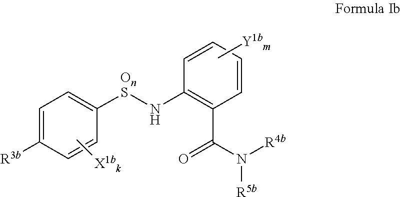

wherein
n is 1 or 2;
each one of R^{4b} and R^{5b} is independently selected from the group consisting of H, alkyl, aryl, arylalkyl, cycloalkyl and haloalkyl, or wherein R^{4b} and R^{5b} together with the nitrogen atom to which they are bound form a ring, which is unsubstituted or substituted with at least one of alkyl, haloalkyl and/or halogen;
each one of X^{1b} and Y^{1b} is independently selected from the group consisting of a halogen and an alkyl;
each one of m and k is 0, 1 or 2; and R^{3b} is selected from hydrogen and an alkyl group.

According to some embodiments, R^{4b} and R^{5b} together with the nitrogen atom to which they are bound form a ring, which is unsubstituted or substituted with at least one of alkyl, haloalkyl and/or halogen. Specifically, the heterocyclic ring formed from NR⁴R⁵ is devoid of aromatic substituents, such as phenyl and substituted phenyl rings, according to some embodiments.

According to some embodiments, R^{4b} and R^{5b} together with the nitrogen atom to which they are bound form a ring, wherein the formed ring is unsubstituted or substituted with at least one alkyl, or haloalkyl. According to some embodiments, the formed ring is unsubstituted or substituted with at least one alkyl group. According to some embodiments, the formed ring is substituted with at least one alkyl group. According to some embodiments, the formed ring is substituted with at least two alkyl groups. According to some embodiments, the formed ring is substituted with two alkyl groups. According to some embodiments, each alkyl group, which serves as substituent to the ring formed from NR^{4b}R^{5b} is a C₁-C₆ unsubstituted alkyl. According to some embodiments, each alkyl group, which serves as substituent to the ring formed from NR^{4b}R^{5b} is a C₁-C₆ linear alkyl.

According to some embodiments, each alkyl group, which serves as substituent to the ring formed from NR^{4b}R^{5b} is a C₁-C₄ unsubstituted alkyl. According to some embodiments, each alkyl group, which serves as substituent to the ring formed from NR^{4b}R^{5b} is a C₁-C₄ linear alkyl. According to some embodiments, each alkyl group, which serves as substituent to the ring formed from NR^{4b}R^{5b} is a C₁-C₂ unsubstituted alkyl. According to some embodiments, each alkyl group, which serves as substituent to the ring formed from NR^{4b}R^{5b} is selected from ethyl and methyl. According to some embodiments, each alkyl group, which serves as substituent to the ring formed from NR^{4b}R^{5b} is a C₁-C₂ linear alkyl. According to some embodiments, R^{4b} and R^{5b} together with the nitrogen atom to which they are bound form a ring, which is substituted with two alkyl groups, wherein each alkyl group is selected from methyl and ethyl.

According to some embodiments, the ring formed from NR^{4b}R^{5b} is an N-heterocycle selected from the group consisting of pyrrolidine, pyrrole, oxazole, succinimide, piperidine, pyridine, pyrimidine, piperazine, morpholine, thiomorpholine, indoline and indole, wherein each N-heterocycle is optionally substituted with one or more substituents selected from the group consisting of alkyl, haloalkyl, and halogen. Each possibility represents a separate embodiment. In particular, the N-heterocycle may be devoid of aromatic substituents, such as phenyl and its derivatives, according to some embodiments. According to some embodiments, the ring formed from NR^{4b}R^{5b} is an N-heterocycle selected from pyrrolidine, pyrrole, oxazole, succinimide, pyridine, pyrimidine, piperazine, morpholine, thiomorpholine, indoline and indole, wherein each N-heterocycle is optionally substituted with one or more substituents selected from the group consisting of alkyl, haloalkyl, and halogen. According to some embodiments, the ring formed from NR^{4b}R^{5b} is an N-heterocycle selected from pyrrolidine, and morpholine, wherein each N-heterocycle is optionally substituted with one or more substituents selected from the group consisting of alkyl, haloalkyl, and halogen. According to some embodiments, the N-heterocycle is substituted with one or more of the substituents as described herein. According to some embodiments, the N-heterocycle is substituted with two or more of the substituents. According to some embodiments, the N-heterocycle is substituted with two of the substituents. According to some embodiments, the substituent is an alkyl. According to some embodiments, the substituent is selected from methyl and ethyl.

According to some embodiments, R^{4b} and R^{5b} together with the nitrogen atom to which they are bound form a ring, wherein the ring is a non-aromatic N-heterocycle. According to some embodiments, the non-aromatic heterocycle is selected from the group consisting of pyrrolidine, succinimide, piperidine, morpholine, thiomorpholine and indoline, wherein each non-aromatic N-heterocycle is optionally substituted with one or more substituents selected from the group consisting of alkyl, haloalkyl, and halogen. Each possibility represents a separate embodiment. In particular, the non-aromatic N-heterocycle may be devoid of aromatic substituents, such as phenyl and its derivatives, according to some embodiments. According to some embodiments, the non-aromatic heterocycle is selected from the group consisting of pyrrolidine, succinimide, morpholine, thiomorpholine and indoline, wherein each non-aromatic N-heterocycle is optionally substituted with one or more substituents selected from the group consisting of alkyl, haloalkyl, and halogen. According to some embodiments, the ring formed from NR⁴R⁵ is a non-aromatic N-heterocycle selected from pyrrolidine, and morpholine, wherein each non-aromatic N-heterocycle is optionally substituted with one or more substituents selected from the group consisting of alkyl, haloalkyl, and halogen. According to some embodiments, the non-aromatic N-heterocycle is substituted with one or more of the substituents. According to some embodiments, the N-heterocycle is substituted with two or more of the substituents. According to some embodiments, the non-aromatic N-heterocycle is substituted with two of the substituents. According to some embodiments, the substituent is an alkyl. According to some embodiments, the substituent is selected from methyl and ethyl.

According to some embodiments, N-heterocycle is morpholine. According to some embodiments, the non-aromatic N-heterocycle is morpholine. According to some embodiments, R^{4b} and R^{5b} together with the nitrogen atom to which they are bound form a ring, wherein the ring is a morpholine, which is optionally substituted with one or more substituents selected from the group consisting of alkyl, haloalkyl, and halogen. Each possibility represents a separate embodiment. According to some embodiments, the morpholine is substituted with one or more of the substituents. According to some embodiments, the morpholine is substituted with two or more of the substituents. According to some embodiments, the morpholine is substituted with two of the substituents. According to some embodiments, the substituent is an alkyl. According to some embodiments, the pyrrolidine is substituted with two alkyl groups. According to some embodiments, each of the alkyl groups is a C₁-C₆ alkyl. According to some embodiments, each of the alkyl groups is a C₁-C₆ unsubstituted alkyl. According to some embodiments, each of the alkyl groups is a C₁-C₆ linear alkyl. According to some embodiments, each of the alkyl groups is a C₁-C₄ alkyl. According to some embodiments, each of the alkyl groups is a C₁-C₄ unsubstituted alkyl. According to some embodiments, each of the alkyl groups is a C₁-C₄ linear alkyl.

According to some embodiments, each of the alkyl groups is selected from the group consisting of methyl, ethyl, n-propyl, isopropyl, n-butyl and tert-butyl. Each possibility represents a separate embodiment. According to some embodiments, each of the alkyl groups is selected from the group consisting of methyl and ethyl.

According to some embodiments, R^{4b} and R^{5b} together with the nitrogen atom to which they are bound form the ring:

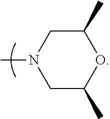

According to some embodiments, Y^{1b} is selected from the group consisting of a halogen and an alkyl. According to some embodiments, when m is 2, each one of Y^{1b} is independently selected from the group consisting of a halogen and an alkyl.

According to some embodiments, m is 0 or 1. According to some embodiments, m is 0.

It is to be understood that when m is 0, the benzene ring, to which the CONR^{4b}R^{5b} is attached, is covalently bonded to four hydrogen atoms, to CONR^{4b}R^{5b} and to the aromatic nitrogen atom.

According to some embodiments, n is 2. It is apparent from the presentation of Formula 1b that when n is 2, a sulfonamide group forms, wherein the sulfonamide nitrogen atom is covalently bonded to the sulfur atom, to one of the benzene rings of Formula Ib, and to a hydrogen atom. It is to be understood by a person skilled in the art that such sulfonamides having a N—H bond are mildly acidic, i.e. they are having a tendency to form a relatively stable —SO₂N⁻— anion, while releasing a proton, depending on the ambient pH. According to some embodiments, the compounds of formula Ib may be provided in a form of a basic salt. Consequently, the phrase “a compound represented by Formula Ib, or a salt thereof” refers mainly to basic salts of the sulfonamide moiety, according to some embodiments. Other types of salts may be associated with one of the substituents of Formula Ib, such as R^{3b}, R^{4b}, R^{5b}, X^{1b} or Y^{1b}. According to some embodiments, the compound of Formula 1b is provided in the form of a salt.

According to some embodiments, X^{1b} is selected from the group consisting of a halogen and an alkyl. According to some embodiments, when k is 2, each one of X^{1b} is independently selected from the group consisting of a halogen and an alkyl. According to some embodiments, X^{1b} is a halogen. According to some embodiments, when k is 2, each one of X^{1b} is independently a halogen.

According to some embodiments, k is 0 or 1. According to some embodiments, k is 0.

According to some embodiments, R^{3b} is a C₁-C₆ alkyl. According to some embodiments, R^{3b} is a C₁-C₄ alkyl. According to some embodiments, R^{3b} is an unsubstituted alkyl. According to some embodiments, R^{3b} is a branched alkyl.

According to some embodiments, R^{3b} is selected from the group consisting of methyl, ethyl, n-propyl, isopropyl, n-butyl, sec-butyl and tert-butyl. Each possibility represents a separate embodiment. According to some embodiments, R^{3b} is selected from the group consisting of methyl and sec-butyl. According to some embodiments, R^{3b} is sec-butyl.

According to some embodiments, the compound of Formula 1b is Compound 1b, as presented herein above or a salt thereof:

According to some embodiments, compound 1b is also referred to herein as compound C5. According to some embodiments, compound 1b also includes any derivatives or salts thereof.

Specifically, according to some embodiments, Compound 1b corresponds to Formula Ib, wherein n is 2; R^{4b} and R^{5b} together with the nitrogen atom to which they are bound form a ring, wherein the ring is a 2,6-dimethylmorpholine having the following formula:

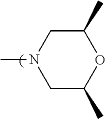

k is 0, such that X^{1b} is absent; m is 0, such that Y^{1b} is absent; and R^{3b} is sec-butyl.

According to some embodiments, the compound is represented by Formula Ic, or a salt thereof:

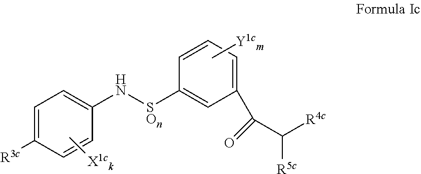

wherein
n is 1 or 2;
each one of R^{4c} and R^{5c} is independently selected from the group consisting of H, alkyl, aryl, arylalkyl, cycloalkyl and haloalkyl, or wherein R^{4c} and R^{5c} together with the nitrogen atom to which they are bound form a ring, wherein the formed ring is unsubstituted or substituted with at least one alkyl, halogen, or haloalkyl;
each one of X^{1c} and Y^{1c} is independently selected from the group consisting of a halogen and an alkyl;
each one of m and k is 0, 1 or 2; and
R^{3c} is selected from hydrogen and an alkyl group.

According to some embodiments, R^{4c} and R^{5c} together with the nitrogen atom to which they are bound form a ring, which is unsubstituted or substituted with at least one of alkyl, haloalkyl and/or halogen. Specifically, the heterocyclic ring formed from NR^{4a}R^{5a} is devoid of aromatic substituents, such as phenyl and substituted phenyl rings, according to some embodiments.

According to some embodiments, R^{4c} and R^{5c} together with the nitrogen atom to which they are bound form a ring, wherein the formed ring is unsubstituted or substituted with at least one alkyl, or haloalkyl. According to some embodiments, the formed ring is unsubstituted or substituted with at least one alkyl group. According to some embodiments, the formed ring is substituted with at least one alkyl group. According to some embodiments, the formed ring is substituted with at least two alkyl groups. According to some embodiments, the formed ring is substituted with two alkyl groups. According to some embodiments, each alkyl group, which serves as substituent to the ring formed from NR^{4c}R^{5c} is a C₁-C₆ unsubstituted alkyl. According to some embodiments, each alkyl group, which serves as substituent to the ring formed from NR^{4c}R^{5c} is a C₁-C₆ linear alkyl. According to some embodiments, each alkyl group, which serves as substituent to the ring formed from NR^{4c}R^{5c} is a C₁-C₄ unsubstituted alkyl. According to some embodiments, each alkyl group, which serves as substituent to the ring formed from NR^{4c}R^{5c} is a C₁-C₄ linear alkyl. According to some embodiments, each alkyl group, which serves as substituent to the ring formed from NR^{4c}R^{5c} is a C₁-C₂ unsubstituted alkyl. According to some embodiments, each alkyl group, which serves as substituent to the ring formed from NR^{4c}R^{5c} is selected from ethyl and methyl. According to some embodiments, each alkyl group, which serves as substituent to the ring formed from NR^{4a}R^{5a} is a C₁-C₂ linear alkyl. According to some embodiments, R^{4c} and R^{5c} together with the nitrogen atom to which they are bound form a ring, which is substituted with two alkyl groups, wherein each alkyl group is selected from methyl and ethyl.

According to some embodiments, the ring is an N-heterocycle selected from the group consisting of pyrrolidine, pyrrole, oxazole, succinimide, piperidine, pyridine, pyrimidine, piperazine, morpholine, thiomorpholine, indoline and indole, wherein each N-heterocycle is optionally substituted with one or more substituents selected from the group consisting of alkyl, haloalkyl, aryl, halogen, hydroxyl, alkoxy group, acyloxy group, carboxy group, carboalkoxy group, amino group, and amido group. Each possibility represents a separate embodiment. According to some embodiments, the N-heterocycle is substituted with one or more of the substituents. According to some embodiments, the N-heterocycle is substituted with two or more of the substituents. According to some embodiments, the N-heterocycle is substituted with two of the substituents. According to some embodiments, the substituent is an alkyl. According to some embodiments, the substituent is selected from methyl and ethyl.

According to some embodiments, R^{4c} and R^{5c} together with the nitrogen atom to which they are bound form a ring, wherein the ring is a non-aromatic N-heterocycle. According to some embodiments, the non-aromatic heterocycle is selected from the group consisting of pyrrolidine, succinimide, piperidine, morpholine, thiomorpholine and indoline, wherein each non-aromatic N-heterocycle is optionally substituted with one or more substituents selected from the group consisting of alkyl, haloalkyl and halogen. Each possibility represents a separate embodiment. In particular, the non-aromatic N-heterocycle may be devoid of aromatic substituents, such as phenyl and its derivatives, according to some embodiments. According to some embodiments, the non-aromatic heterocycle is selected from the group consisting of pyrrolidine, succinimide, morpholine, thiomorpholine and indoline, wherein each non-aromatic N-heterocycle is optionally substituted with one or more substituents selected from the group consisting of alkyl, haloalkyl, and halogen. According to some embodiments, the ring formed from NR⁴R⁵ is a non-aromatic N-heterocycle selected from pyrrolidine, and morpholine, wherein each non-aromatic N-heterocycle is optionally substituted with one or more substituents selected from the group consisting of alkyl, haloalkyl, and halogen. According to some embodiments, the non-aromatic N-heterocycle is substituted with one or more of the substituents. According to some embodiments, the N-heterocycle is substituted with two or more of the substituents. According to some embodiments, the non-aromatic N-heterocycle is substituted with two of the substituents. According to some embodiments, the substituent is an alkyl. According to some embodiments, the substituent is selected from methyl and ethyl.

According to some embodiments, N-heterocycle is morpholine. According to some embodiments, the non-aromatic N-heterocycle is morpholine. According to some embodiments, R^{4c} and R^{5c} together with the nitrogen atom to which they are bound form a ring, wherein the ring is a morpholine, which is optionally substituted with one or more substituents selected from the group consisting of alkyl, haloalkyl, and halogen. Each possibility represents a separate embodiment. According to some embodiments, the morpholine is substituted with one or more of the substituents. According to some embodiments, the morpholine is substituted with two or more of the substituents. According to some embodiments, the morpholine is substituted with two of the substituents. According to some embodiments, the substituent is an alkyl. According to some embodiments, the pyrrolidine is substituted with two alkyl groups. According to some embodiments, each of the alkyl groups is a C₁-C₆ alkyl. According to some embodiments, each of the alkyl groups is a C₁-C₆ unsubstituted alkyl. According to some embodiments, each of the alkyl groups is a C₁-C₆ linear alkyl. According to some embodiments, each of the alkyl groups is a C₁-C₄ alkyl. According to some embodiments, each of the alkyl groups is a C₁-C₄ unsubstituted alkyl. According to some embodiments, each of the alkyl groups is a C₁-C₄ linear alkyl.

According to some embodiments, each of the alkyl groups is selected from the group consisting of methyl, ethyl, n-propyl, isopropyl, n-butyl and tert-butyl. Each possibility represents a separate embodiment. According to some embodiments, each of the alkyl groups is selected from the group consisting of methyl and ethyl.

According to some embodiments, R^{4c} and R^{5c} together with the nitrogen atom to which they are bound form the ring:

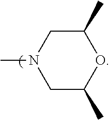

According to some embodiments, Y^{1c} is selected from the group consisting of a halogen and an alkyl. According to some embodiments, when m is 2, each one of Y^{1c} is independently selected from the group consisting of a halogen and an alkyl.

According to some embodiments, m is 1 or 2. According to some embodiments, m is 2. According to some embodiments, m is 2, and each one of Y^{1c} is independently a halogen. According to some embodiments, the halogen is chlorine. According to some embodiments, one of the chlorine atoms is positioned ortho to the carbonyl group and the other chlorine atom is positioned ortho to the sulfur atom. According to some embodiments, one of the chlorine atoms is positioned ortho to the sulfur atom and the other chlorine atom is positioned para to the carbonyl group.

According to some embodiments, n is 2. It is apparent from the presentation of Formula Ic that when n is 2, a sulfonamide group forms, which is deprotonizable as detailed herein. According to some embodiments, the compounds of formula Ic may be provided in a form of a basic salt. Consequently, the phrase “a compound represented by Formula Ic, or a salt thereof” refers mainly to basic salts of the sulfonamide moiety, according to some embodiments. Other types of salts may be associated with one of the substituents of Formula Ic, such as R^{3c}, R^{4c}, R^{5c}, X^{1c} or Y^{1c}. According to some embodiments, the compound of Formula Ic is provided in the form of a salt.

According to some embodiments, X^{1c} is selected from the group consisting of a halogen and an alkyl. According to some embodiments, when k is 2, each one of X^{1c} is independently selected from the group consisting of a halogen and an alkyl. According to some embodiments, X^{1c} is a halogen. According to some embodiments, when k is 2, each one of X¹ is independently a halogen. According to some embodiments, k is 0 or 1. According to some embodiments, k is 1. According to some embodiments, k is 1 and X^{1c} is a halogen. According to some embodiments, X^{1c} is selected from the group consisting of fluorine, chlorine and bromine. Each possibility represents a separate embodiment. According to some embodiments, X^{1c} is selected from the group consisting of fluorine and chlorine. According to some embodiments, X^{1c} is fluorine.

According to some embodiments k is 1 and X^{1c} is positioned ortho to the aromatic nitrogen atom. According to some embodiments, k is 1. According to some embodiments, k is 1 and X^{1a} is a halogen positioned ortho to the aromatic nitrogen atom. According to some embodiments, k is 1.

According to some embodiments, k is 1 and X^{1c} is a fluorine atom positioned ortho to the aromatic nitrogen atom.

According to some embodiments, R³ is H.

According to some embodiments, the compound of Formula Ic is Compound 1c, as presented herein above, or a salt thereof:

According to some embodiments, compound 1c is also referred to herein as compound C14.

According to some embodiments, compound 1c also includes any derivatives or salts thereof.

Specifically, according to some embodiments, Compound 1c corresponds to Formula Ic, wherein n is 2; R^{4c} and R^{5c} together with the nitrogen atom to which they are bound form a ring, wherein the ring is a 2,6-dimethylmorpholine having the following formula:

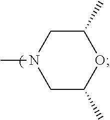

k is 1; X^{1c} is fluorine positioned ortho to the aromatic nitrogen; m is 2; one Y^{1c} is chlorine positioned ortho to the aromatic sulfur and one Y^{1c} is chlorine positioned ortho to the aromatic carbonyl group; and R^{3c} is H.

### Formula II

According to some embodiments, the compound is represented by Formula II, as presented above, an ester or a salt thereof, wherein R¹¹ is CH₂R¹³, wherein R¹³ is selected from the group consisting of hydrogen, an alkyl and an aryl; R¹² is selected from the group consisting of hydrogen and an alkyl; each one of X¹¹ and Y¹¹ is independently selected from the group consisting of a halogen and an alkyl; and each one of p, r and q is 0, 1 or 2.

It is to be understood that the double bond configuration of Formula II may be either E or Z. According to some embodiments. According to some embodiments, the benzene ring is cis to the benzothiazole ring. According to some embodiments. According to some embodiments, the benzene ring is trans to the benzothiazole ring. It is to be understood that the chemical structure of a compound of Formula II, wherein benzene ring is cis to the benzothiazole ring is represented by Formula II-cis, and that the chemical structure of a compound of Formula II, wherein benzene ring is trans to the benzothiazole ring is represented by Formula II-trans. Thus, according to some embodiments, the compound is represented by Formula II-cis, an ester or a salt thereof. According to some embodiments, the compound is represented by Formula II-trans, an ester or a salt thereof:

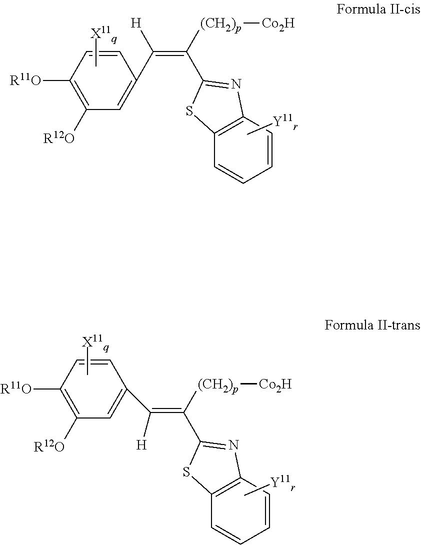

It is to be understood by a person skilled in the art that carboxylic acids, such as the carboxylic acid of Formula II (and Formulas IIa and IIb below) have a tendency to form a relatively stable —CO₂ anion, while releasing a proton, depending on the ambient pH. According to some embodiments, the compounds of formula II may be provided in a form of a basic salt. Consequently, the phrase “a compound represented by Formula II, or a salt thereof” refers mainly to basic salts of the carboxylic acid moiety, according to some embodiments. Other types of salts may be associated with one of the substituents of Formula II.

Additionally, it is to be understood by a person skilled in the art that carboxylic acids, such as the carboxylic acid of Formula II (and Formulas IIa and IIb below) may be esterified to form an ester. Consequently, the phrase “a compound represented by Formula II, or an ester thereof” refers mainly to esters of the carboxylic moiety, according to some embodiments. Other types of esters may be associated with one of the substituents of Formula II.

According to some embodiments, the compound of Formula II is provided in the form of a salt. According to some embodiments, the compound of Formula II is provided in the form of an ester.

According to some embodiments, R¹³ is selected from the group consisting of hydrogen, an alkyl and an aryl. According to some embodiments, R¹³ is an aryl group, optionally substituted with one or more substituents selected from the group consisting of alkyl, haloalkyl, aryl, halogen, hydroxyl, alkoxy group, acyloxy group, carboxy group, carboalkoxy group, amino group, and amido group. Each possibility represents a separate embodiment. According to some embodiments, R¹³ is a phenyl group, optionally substituted with one or more substituents selected from the group consisting of alkyl, haloalkyl, aryl, halogen, hydroxyl, alkoxy group, acyloxy group, carboxy group, carboalkoxy group, amino group, and amido group. Each possibility represents a separate embodiment. According to some embodiments, R¹³ is a phenyl group, optionally substituted with one or more substituents selected from the group consisting of alkyl, hydroxy, haloalkyl and halogen. According to some embodiments, R¹³ is a phenyl group, optionally substituted with one or more substituents selected from the group consisting of haloalkyl and halogen. According to some embodiments, the substituent is a haloalkyl. According to some embodiments, the haloalkyl is a fluoroalkyl. According to some embodiments, the haloalkyl is trifluoromethyl. According to some embodiments, substituent is a halogen. According to some embodiments, the halogen is fluorine.

According to some embodiments, R¹³ is an unsubstituted phenyl group. According to some embodiments, R¹¹ is benzyl. According to some embodiments, R¹³ is a phenyl group substituted with one substituent selected from the group consisting of alkyl, haloalkyl, aryl, halogen, hydroxyl, alkoxy group, acyloxy group, carboxy group, carboalkoxy group, amino group and amido group. According to some embodiments, R¹³ is a phenyl group substituted with one substituent selected from the group consisting of alkyl, hydroxy, haloalkyl and halogen. According to some embodiments, R¹³ is a phenyl group substituted with one substituent selected from the group consisting of haloalkyl and halogen. According to some embodiments, the substituent is a haloalkyl. According to some embodiments, the haloalkyl is a fluoroalkyl. According to some embodiments, the haloalkyl is trifluoromethyl. According to some embodiments, substituent is a halogen. According to some embodiments, the halogen is fluorine.

According to some embodiments, R¹³ is selected from phenyl, 3-trifluoromethylphenyl and 4-fluorophenyl. According to some embodiments, R¹¹ is selected from benzyl, 3-trifluoromethylbenzyl and 4-fluorobenzyl. According to some embodiments, R¹³ is selected from 3-trifluoromethylphenyl and 4-fluorophenyl. According to some embodiments, R¹¹ is selected from 3-trifluoromethylbenzyl and 4-fluorobenzyl. According to some embodiments, R¹³ is 3-trifluoromethylphenyl. According to some embodiments, R¹¹ is 3-trifluoromethylbenzyl. According to some embodiments, R¹³ is 4-fluorophenyl. According to some embodiments, R¹¹ is 4-fluorobenzyl.

According to some embodiments, R¹² is selected from the group consisting of hydrogen and an alkyl, wherein the alkyl is optionally substituted with one or more substituents selected from the group consisting of alkyl, haloalkyl, aryl, halogen, hydroxyl, alkoxy group, acyloxy group, carboxy group, carboalkoxy group, amino group, and amido group. Each possibility represents a separate embodiment.

According to some embodiments, R¹² is an alkyl. According to some embodiments, R¹² is a C₁-C₆ alkyl. According to some embodiments, R¹² is a C₁-C₄ alkyl. According to some embodiments, R¹² is an unsubstituted alkyl. According to some embodiments, R¹² is a linear alkyl. According to some embodiments, R¹² is selected from the group consisting of methyl, ethyl, n-propyl, isopropyl, n-butyl and tert-butyl. Each possibility represents a separate embodiment. According to some embodiments, R¹² is methyl or ethyl. According to some embodiments, R¹² is methyl.

According to some embodiments, p is 1 or 2. According to some embodiments, p is 1. According to some embodiments, p is 2.

According to some embodiments, X¹¹ is selected from the group consisting of a halogen and an alkyl. According to some embodiments, when q is 2, each one of X¹¹ is independently selected from the group consisting of a halogen and an alkyl. According to some embodiments, X¹¹ is a halogen. According to some embodiments, when q is 2, each one of X¹¹ is independently a halogen.

According to some embodiments, q is 0 or 1. According to some embodiments, q is 0. It is to be understood that when q is 0 X¹¹ is absent.

According to some embodiments, Y¹¹ is selected from the group consisting of a halogen and an alkyl. According to some embodiments, when r is 2, each one of Y¹¹ is independently selected from the group consisting of a halogen and an alkyl. According to some embodiments, Y¹¹ is a halogen. According to some embodiments, when r is 2, each one of Y¹¹ is independently a halogen.

According to some embodiments, r is 0 or 1. According to some embodiments, r is 0. It is to be understood that when r is 0 Y¹¹ is absent.

According to some embodiments, the compound is selected from Compound 2a, Compound 2b, Compound 2c, as presented above, esters and salts thereof.

According to some embodiments, Compound 2a is also referred to herein as compound C17. According to some embodiments, Compound 2a also includes any derivatives or salts thereof. The generic name of Compound 2a is (Z)-3-(benzo[d]thiazol-2-yl)-4-(3-methoxy-4-((3-(trifluoromethyl)benzyl)oxy)phenyl)but-3-enoic acid.

Specifically, according to some embodiments, Compound 2a corresponds to Formula II, wherein the double bond is in a Z configuration; R¹¹ is 3-trifluormethylbenzyl; R¹² is methyl; p is 1; r is 0, such that Y¹¹ is absent; and q is 0, such that X¹¹ is absent.

According to some embodiments, Compound 2b is also referred to herein as compound C15. According to some embodiments, Compound 2b also includes any derivatives or salts thereof. The generic name of Compound 2b is (E)-4-(benzo[d]thiazol-2-yl)-5-(4-(benzyloxy)-3-methoxyphenyl)pent-4-enoic acid.

Specifically, according to some embodiments, Compound 2b corresponds to Formula II, wherein the double bond is in an E configuration R¹¹ is benzyl; R¹² is methyl; p is 2; r is 0, such that Y¹¹ is absent; and q is 0, such that X¹¹ is absent.

According to some embodiments, Compound 2c is also referred to herein as compound C20. According to some embodiments, Compound 2c also includes any derivatives or salts thereof. The generic name of Compound 2c is (E)-4-(benzo[d]thiazol-2-yl)-5-(4-((4-fluorobenzyl)oxy)-3-methoxyphenyl)pent-4-enoic acid.

Specifically, according to some embodiments, Compound 2c corresponds to Formula II, wherein the double bond is in an E configuration; R¹¹ is 4-fluorobenzyl; R¹² is methyl; r is 0, such that Y¹¹ is absent; and q is 0, such that X¹¹ is absent.

According to some embodiments, the compound is selected from Compound 2a, Compound 2b or salts thereof. According to some embodiments, the compound is selected from Compound 2a, Compound 2c or salts thereof. According to some embodiments, the compound is selected from Compound 2b, Compound 2c or salts thereof. According to some embodiments, the compound is Compound 2a, an ester or a salt thereof. According to some embodiments, the compound is Compound 2b, an ester or a salt thereof. According to some embodiments, the compound is Compound 2c, an ester or a salt thereof.

According to some embodiments, the compound is represented by Formula IIa, an ester or a salt thereof.

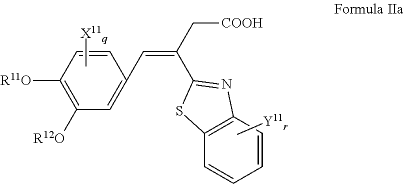

wherein R^{11a} is CH₂R^{13a}, wherein R^{13a} is selected from the group consisting of hydrogen, an alkyl and an aryl; R^{12a} is selected from the group consisting of hydrogen and an alkyl; each one of X¹¹ and Y¹¹ is independently selected from the group consisting of a halogen and an alkyl; and each one of p, r and q is 0, 1 or 2.

According to some embodiments, R^{13a} is selected from the group consisting of hydrogen, an alkyl and an aryl. According to some embodiments, R^{13a} is an aryl group, optionally substituted with one or more substituents selected from the group consisting of alkyl, haloalkyl, aryl, halogen, hydroxyl, alkoxy group, acyloxy group, carboxy group, carboalkoxy group, amino group, and amido group. Each possibility represents a separate embodiment. According to some embodiments, R^{13a} is a phenyl group, optionally substituted with one or more substituents selected from the group consisting of alkyl, haloalkyl, aryl, halogen, hydroxyl, alkoxy group, acyloxy group, carboxy group, carboalkoxy group, amino group, and amido group. Each possibility represents a separate embodiment. According to some embodiments, R^{13a} is a phenyl group, optionally substituted with one or more substituents selected from the group consisting of alkyl, hydroxy, haloalkyl and halogen. According to some embodiments, R^{13a} is a phenyl group, optionally substituted with one or more substituents selected from the group consisting of haloalkyl and halogen. According to some embodiments, the substituent is a haloalkyl. According to some embodiments, the haloalkyl is a fluoroalkyl. According to some embodiments, the haloalkyl is trifluoromethyl. According to some embodiments, substituent is a halogen. According to some embodiments, the halogen is fluorine.

According to some embodiments, R^{13a} is a phenyl group substituted with one substituent selected from the group consisting of alkyl, haloalkyl, aryl, halogen, hydroxyl, alkoxy group, acyloxy group, carboxy group, carboalkoxy group, amino group and amido group. According to some embodiments, R^{13a} is a phenyl group substituted with one substituent selected from the group consisting of alkyl, hydroxy, haloalkyl and halogen. According to some embodiments, R^{13a} is a phenyl group substituted with one substituent selected from the group consisting of haloalkyl and halogen. According to some embodiments, the substituent is a haloalkyl. According to some embodiments, the haloalkyl is selected from the group consisting of fluoromethyl, difluoromethyl and trifluoromethyl. According to some embodiments, the haloalkyl is a polyfluoroalkyl. According to some embodiments, the haloalkyl is a perfluoroalkyl. According to some embodiments, the haloalkyl is trifluoromethyl.

According to some embodiments, R^{13a} is selected from 3-trifluoromethylphenyl and 4-fluorophenyl. According to some embodiments, R^{11a} is selected from 3-trifluoromethylbenzyl and 4-fluorobenzyl. According to some embodiments, R^{13a} is 3-trifluoromethylphenyl. According to some embodiments, R^{11a} is 3-trifluoromethylbenzyl.

According to some embodiments, R^{12a} is selected from the group consisting of hydrogen and an alkyl, wherein the alkyl is optionally substituted with one or more substituents selected from the group consisting of alkyl, haloalkyl, aryl, halogen, hydroxyl, alkoxy group, acyloxy group, carboxy group, carboalkoxy group, amino group, and amido group. Each possibility represents a separate embodiment. According to some embodiments, R^{12a} is an alkyl. According to some embodiments, R^{12a} is a C₁-C₆ alkyl. According to some embodiments, R^{12a} is a C₁-C₄ alkyl. According to some embodiments, R^{12a} is an unsubstituted alkyl. According to some embodiments, R^{12a} is a linear alkyl. According to some embodiments, R^{12a} is selected from the group consisting of methyl, ethyl, n-propyl, isopropyl, n-butyl and tert-butyl. Each possibility represents a separate embodiment. According to some embodiments, R¹² is methyl or ethyl. According to some embodiments, R^{12a} is methyl.

According to some embodiments, X^{11a} is selected from the group consisting of a halogen and an alkyl. According to some embodiments, when q is 2, each one of X^{11a} is independently selected from the group consisting of a halogen and an alkyl. According to some embodiments, X^{11a} is a halogen. According to some embodiments, when q is 2, each one of X^{11a} is independently a halogen. According to some embodiments, q is 0 or 1. According to some embodiments, q is 0. It is to be understood that when q is 0 X^{11a} is absent. According to some embodiments, Y^{11a} is selected from the group consisting of a halogen and an alkyl. According to some embodiments, when r is 2, each one of Y^{11a} is independently selected from the group consisting of a halogen and an alkyl. According to some embodiments, Y^{11a} is a halogen. According to some embodiments, when r is 2, each one of Y^{11a} is independently a halogen.

According to some embodiments, r is 0 or 1. According to some embodiments, r is 0. It is to be understood that when r is 0 Y^{11a} is absent.

According to some embodiments, the compound of Formula IIa is Compound 2a, as presented herein above, an ester or a salt thereof.

Specifically, according to some embodiments, Compound 2a corresponds to Formula IIa, wherein R^{11a} is 3-trifluormethylbenzyl; R^{12a} is methyl; r is 0, such that Y¹¹ is absent; and q is 0, such that X¹¹ is absent.

According to some embodiments, the compound is represented by Formula IIb, or a salt thereof:

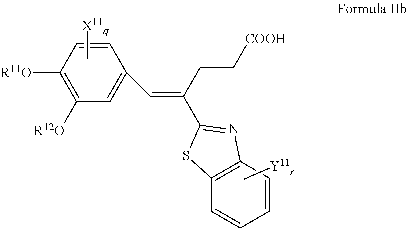

wherein R^{11b} is CH₂R^{13b}, wherein R^{13b} is selected from the group consisting of hydrogen, an alkyl and an aryl; R^{12b} is selected from the group consisting of hydrogen and an alkyl; each one of X¹¹ and Y¹¹ is independently selected from the group consisting of a halogen and an alkyl; and each one of p, r and q is 0, 1 or 2.

According to some embodiments, R^{13b} is selected from the group consisting of hydrogen, an alkyl and an aryl. According to some embodiments, R^{13b} is an aryl group, optionally substituted with one or more substituents selected from the group consisting of alkyl, haloalkyl, aryl, halogen, hydroxyl, alkoxy group, acyloxy group, carboxy group, carboalkoxy group, amino group, and amido group. Each possibility represents a separate embodiment. According to some embodiments, R^{13b} is a phenyl group, optionally substituted with one or more substituents selected from the group consisting of alkyl, haloalkyl, aryl, halogen, hydroxyl, alkoxy group, acyloxy group, carboxy group, carboalkoxy group, amino group, and amido group. Each possibility represents a separate embodiment. According to some embodiments, R^{13b} is a phenyl group, optionally substituted with one or more substituents selected from the group consisting of alkyl, hydroxy, haloalkyl and halogen. According to some embodiments, R^{13b} is a phenyl group, optionally substituted with one or more substituents selected from the group consisting of haloalkyl and halogen. According to some embodiments, the substituent is a haloalkyl. According to some embodiments, the haloalkyl is a fluoroalkyl. According to some embodiments, the halogen is fluorine. According to some embodiments, R^{13b} is an unsubstituted aryl. According to some embodiments, R^{13b} is an unsubstituted phenyl.

According to some embodiments, R^{13b} is selected from phenyl and 4-fluorophenyl. According to some embodiments, R^{13b} is selected from benzyl and 4-fluorobenzyl. According to some embodiments, R^{13b} is phenyl. According to some embodiments, R^{13b} is benzyl. According to some embodiments, R^{13b} is a fluorophenyl. According to some embodiments, R^{13b} is 4-fluorophenyl.

According to some embodiments, R^{13b} is 4-fluorobenzyl.

According to some embodiments, R^{12b} is selected from the group consisting of hydrogen and an alkyl, wherein the alkyl is optionally substituted with one or more substituents selected from the group consisting of alkyl, haloalkyl, aryl, halogen, hydroxyl, alkoxy group, acyloxy group, carboxy group, carboalkoxy group, amino group, and amido group. Each possibility represents a separate embodiment. According to some embodiments, R^{12b} is an alkyl. According to some embodiments, R^{12b} is a C₁-C₆ alkyl. According to some embodiments, R^{12b} is a C₁-C₄ alkyl. According to some embodiments, R^{12b} is an unsubstituted alkyl. According to some embodiments, R^{12b} is a linear alkyl. According to some embodiments, R^{12b} is selected from the group consisting of methyl, ethyl, n-propyl, isopropyl, n-butyl and tert-butyl. Each possibility represents a separate embodiment. According to some embodiments, R^{12b} is methyl or ethyl. According to some embodiments, R^{12b} is methyl.

According to some embodiments, X¹¹ is selected from the group consisting of a halogen and an alkyl. According to some embodiments, when q is 2, each one of X¹¹ is independently selected from the group consisting of a halogen and an alkyl. According to some embodiments, X¹¹ is a halogen. According to some embodiments, when q is 2, each one of X¹¹ is independently a halogen.

According to some embodiments, q is 0 or 1. According to some embodiments, q is 0. It is to be understood that when q is 0 X¹¹ is absent.

According to some embodiments, Y¹¹ is selected from the group consisting of a halogen and an alkyl. According to some embodiments, when r is 2, each one of Y¹¹ is independently selected from the group consisting of a halogen and an alkyl. According to some embodiments, Y¹¹ is a halogen. According to some embodiments, when r is 2, each one of Y¹¹ is independently a halogen.

According to some embodiments, r is 0 or 1. According to some embodiments, r is 0. It is to be understood that when r is 0 Y¹¹ is absent.

According to some embodiments, the compound of Formula IIb is selected from Compound 2b, Compound 2c and salts thereof, as presented above.

Specifically, according to some embodiments, Compound 2b corresponds to Formula IIb, wherein R^{11b} is benzyl; R^{12b} is methyl; r is 0, such that Y¹¹ is absent; and q is 0, such that X¹¹ is absent.

Specifically, according to some embodiments, Compound 2c corresponds to Formula IIb, wherein R^{11b} is 4-fluorobenzyl; R^{12b} is methyl; r is 0, such that Y¹¹ is absent; and q is 0, such that X¹¹ is absent.

According to some embodiments, the compound of Formula IIb is Compound 2b, an ester or a salt thereof. According to some embodiments, the compound of Formula IIb is Compound 2c, an ester or a salt thereof.

According to some embodiments, the compound is represented by Formula III, presented herein above, an ester or a salt thereof, wherein u is 1 or 2; each X²¹ is selected from the group consisting of OH, OR²² and halogen, wherein R²² is selected from the group consisting of unsubstituted alkyl, unsubstituted aryl, haloalkyl, alkoxyalkyl, hydroxyalkyl and haloaryl; each one of s and t is independently 0, 1 or 2; and R²¹ is selected from the group consisting of unsubstituted aryl, haloaryl, alkoxyaryl, alkylaryl, unsubstituted heteroaryl, haloheteroaryl, alkoxyheteroaryl and alkylheteroaryl.

It is to be understood by a person skilled in the art that carboxylic acids, such as the carboxylic acid of Formula III (and below) have a tendency to form a relatively stable —CO₂ anion, while releasing a proton, depending on the ambient pH. According to some embodiments, the compounds of formula II may be provided in a form of a basic salt. Also, it is to be understood that the triazole ring of Formula III may act as a base to form a respective protonated cation. Consequently, the phrase “a compound represented by Formula III, or a salt thereof” refers mainly to basic salts of the carboxylic acid moiety and/or to acidic salts of the triazole moiety, according to some embodiments. Other types of salts may be associated with one of the substituents of Formula III.

Additionally, it is to be understood by a person skilled in the art that carboxylic acids, such as the carboxylic acid of Formula III may be esterified to form an ester. Consequently, the phrase “a compound represented by Formula III, or an ester thereof” refers mainly to esters of the carboxylic moiety, according to some embodiments. Other types of esters may be associated with one of the substituents of Formula III.

According to some embodiments, the compound of Formula III is provided in the form of a salt. According to some embodiments, the compound of Formula III is provided in the form of an ester.

According to some embodiments, each X²¹ is selected from the group consisting of OH and OR²². According to some embodiments, each X²¹ is OR²².

According to some embodiments, R²² is selected from the group consisting of unsubstituted alkyl, haloalkyl, alkoxyalkyl and hydroxyalkyl. According to some embodiments, R²² is an unsubstituted alkyl. According to some embodiments, R²² is selected from the group consisting of branched alkyl and linear alkyl. According to some embodiments, R²² is a linear alkyl. According to some embodiments, R²² is a C₁-C₄ alkyl. According to some embodiments, R²² is a C₁-C₄ unsubstituted alkyl. According to some embodiments, R²² is a C₁-C₄ unsubstituted linear alkyl. According to some embodiments, R²² is selected from the group consisting of methyl, ethyl, n-propyl, isopropyl, n-butyl, sec-butyl, tert-butyl, pentyl, hexyl, phenyl and benzyl. According to some embodiments, R²² is selected from the group consisting of methyl, ethyl, n-propyl, isopropyl, n-butyl, sec-butyl, and tert-butyl. According to some embodiments, R²² is selected from the group consisting of methyl, ethyl, n-propyl and isopropyl. According to some embodiments, R²² is ethyl or methyl. According to some embodiments, R²² is methyl.

According to some embodiments, each X²¹ is OMe.

According to some embodiments, u is 2. According to some embodiments, u is 2 and each one of the 2 X²¹ is OMe. According to some embodiments, u is 2, one of the two X²¹ is OMe positioned para to the carbon atom a to nitrogen, and the other of the two X²¹ is OMe positioned meta to the carbon atom a to nitrogen.

According to some embodiments, s is 1. According to some embodiments, t is 1.

According to some embodiments, R²¹ is selected from the group consisting of unsubstituted aryl, haloaryl, alkoxyaryl and alkylaryl. According to some embodiments, R²¹ is a haloaryl. According to some embodiments, R²¹ is a fluoroaryl. According to some embodiments, R²¹ is a phenyl group optionally substituted by one or more halogens. According to some embodiments, R²¹ is a fluorophenyl. According to some embodiments, R²¹ is 3-fluorophenyl.

According to some embodiments, the compound is Compound 3, as presented above, an ester or a salt thereof.

According to some embodiments, Compound 3 is also referred to herein as compound C12. According to some embodiments, Compound 3 also includes any derivatives, ester or salts thereof. The generic name of Compound 3 is (R)-3-(3,4-dimethoxyphenyl)-3-(1-(3-fluorobenzyl)-1H-1,2,3-triazole-4-carboxamido)propanoic acid.

Specifically, according to some embodiments, Compound 3 corresponds to Formula III, wherein u is 2; each of X²¹ is OMe, wherein one OMe is positioned para to the carbon atom a to nitrogen, and the other OMe is positioned meta to the carbon atom a to nitrogen; s is 1; and t is 1.

According to some embodiments, the compound is Compound 4a or a salt thereof. According to some embodiments, the compound is Compound 4b or a salt thereof.

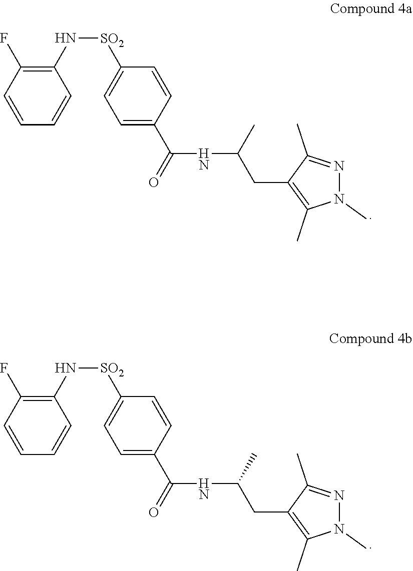 [SMILES: Cc1nn(C)c(C)c1CC(C)NC(=O)c1ccc(S(=O)(=O)Nc2ccccc2F)cc1 ; Cc1nn(C)c(C)c1C[C@@H](C)NC(=O)c1ccc(S(=O)(=O)Nc2ccccc2F)cc1]

Specifically, Compound 4b is similar to Compound 4a, with a stereocenter defined.

According to some embodiments, Compound 4b is also referred to herein as compound C9. According to some embodiments, Compound 4b also includes any derivatives or salts thereof. The generic name of Compound 4b is (R)-4-(N-(2-fluorophenyl)sulfamoyl)-N-(1-(1,3,5-trimethyl-1H-pyrazol-4-yl)propan-2-yl)benzamide.

It is to be understood by a person skilled in the art that sulfonamides having a N—H bond are mildly acidic, i.e. they are having a tendency to form a relatively stable —SO₂N⁻— anion, while releasing a proton, depending on the ambient pH. According to some embodiments, Compounds 4a and 4b may be provided in a form of a basic salt. Also, it is to be understood that the triazole ring of Compounds 4a and 4b may act as a base to form a respective protonated cation. Consequently, the phrases “Compound 4a or a salt thereof” and “Compound 4b or a salt thereof” refer mainly to basic salts of the sulfonamide moiety and/or to acidic salts of the triazole moiety, according to some embodiments.

According to some embodiments, the compound is Compound 5a or a salt thereof. According to some embodiments, the compound is Compound 5b or a salt thereof.

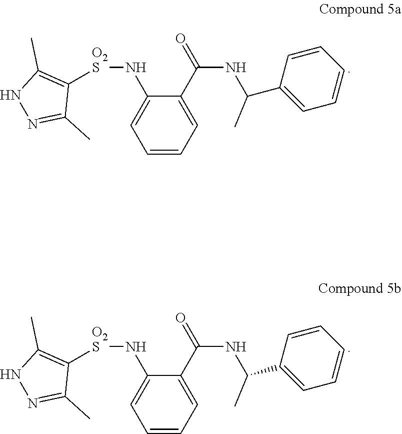 [SMILES: Cc1n[nH]c(C)c1S(=O)(=O)Nc1ccccc1C(=O)NC(C)c1ccccc1 ; Cc1n[nH]c(C)c1S(=O)(=O)Nc1ccccc1C(=O)N[C@@H](C)c1ccccc1]

Specifically, Compound 5b is similar to Compound 5a, with a stereocenter defined.

According to some embodiments, Compound 5b is also referred to herein as compound C4. According to some embodiments, Compound 5b also includes any derivatives or salts thereof. The generic name of Compound 5b is (S)-2-((3,5-dimethyl-1H-pyrazole)-4-sulfonamido)-N-(1-phenylethyl)benzamide.

It is to be understood by a person skilled in the art that sulfonamides having a N—H bond are mildly acidic, i.e. they are having a tendency to form a relatively stable —SO₂N⁻— anion, while releasing a proton, depending on the ambient pH. According to some embodiments, Compounds 5a and 5b may be provided in a form of a basic salt.

The term “alkyl” refers to a substituted or an unsubstituted, an unbranched or a branched alkyl-group, that contains of 1 to 20 carbon atoms, preferably 1 to 12 carbon atoms, especially preferably 1 to 7 carbon atoms, for example the methyl, ethyl, isopropyl, isobutyl, tert-butyl, n-hexyl, 2,2-di-methylbutyl, n-octyl, benzyl, fluorobenzyl, flouromethyl, chloromethyl or trifluoromethyl. The term “arylalkyl” refers to alkyl groups substituted with at least one aryl group. For example, the CH(Ph)CH₃ group is a representative arylalkyl. Similarly, the term “haloalkyl” refers to alkyl groups substituted with one or more halogens. For example, the CH₂CHClCH₃ and the CF₃ groups are representative haloalkyls.

The terms “aryl” and “Ar” as used herein, are interchangeable and refer to aromatic groups, such as phenyl, naphthyl and phenanthrenyl, which may optionally contain one or more substituents, such as alkyl, alkoxy, alkylthio, halo, hydroxy, amino and the like.

The term “halogen” represents fluorine, chlorine, bromine, iodine, preferably fluorine.

As used herein, the singular form “a,” “an,” and “the” include plural references unless the context clearly dictates otherwise. For example, the term “a compound” or “at least one compound” may include a plurality of compounds, including mixtures thereof.

The term “comprising” means that other steps and ingredients that do not affect the final result can be added. This term encompasses the terms “consisting of” and “consisting essentially of”.

### Pharmaceutical Compositions

According to certain aspects and embodiments, the invention relates to a pharmaceutical composition comprising a therapeutically effective amount of at least one compound of the invention, and a pharmaceutically acceptable carrier or excipient. In a particular embodiment, the pharmaceutical composition comprises the compound as the sole active ingredient. In another particular embodiment, said composition comprises two or more compounds of the invention as the sole active ingredients.

In some embodiments, provided is a pharmaceutical grade purity of one or more compounds selected from the group consisting of Compound 1a, Compound 1b, Compound 1c, Compound 2a, Compound 2b, Compound 2c, and Compound 3, esters and salts thereof, said composition further comprising a pharmaceutically acceptable carrier, excipient or diluent. As used herein, the term “Pharmaceutical grade purity” refers to the degree of chemical purity of a compound that is suitable for drug or medicinal administration. In some embodiments, pharmaceutical grade purity is ≥95%. In some embodiments, a pharmaceutical grade purity is ACS grade purity, which meets or exceeds purity standards set by the American Chemical Society (ACS). In some embodiments, a pharmaceutical grade purity is “reagent grade” purity, which is generally equal to ACS grade (≥95%) and is acceptable for food, drug, or medicinal use.

Suitable pharmaceutically acceptable carriers or excipients include, but are not limited to, a binder, a filler, a diluent, a surfactant or emulsifier, a glidant or lubricant, buffering or pH adjusting agent, a tonicity enhancing agent, a wetting agent, a preservative, an antioxidant, a flavoring agent, a colorant, and a mixture or combination thereof. Each possibility represents a separate embodiment. Suitable binders include, but are not limited to, polyvinylpyrrolidone, copovidone, hydroxypropyl methylcellulose, starch, and gelatin. Each possibility represents a separate embodiment. Suitable fillers include, but are not limited to, sugars such as lactose, sucrose, mannitol or sorbitol and derivatives therefore (e.g. amino sugars), ethylcellulose, microcrystalline cellulose, and silicified microcrystalline cellulose. Each possibility represents a separate embodiment. Suitable lubricants include, but are not limited to, sodium stearyl fumarate, stearic acid, polyethylene glycol or stearates, such as magnesium stearate. Each possibility represents a separate embodiment. Suitable diluents include, but are not limited to, dicalcium phosphate dihydrate, sugars, lactose, calcium phosphate, cellulose, kaolin, mannitol, sodium chloride, and dry starch. Each possibility represents a separate embodiment.

Suitable surfactants or emulsifiers include, but are not limited to, polyvinyl alcohol (PVA), polysorbate, polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil. Each possibility represents a separate embodiment. Suitable glidants or lubricants include, but are not limited to, colloidal silicon dioxide, magnesium stearate, talc, and mineral oil. Each possibility represents a separate embodiment. Suitable buffering or pH adjusting agents include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide, and magnesium hydroxide. Each possibility represents a separate embodiment. Suitable tonicity enhancing agents include, but are not limited to, ionic and non-ionic agents such as, alkali metal or alkaline earth metal halides, urea, glycerol, sorbitol, mannitol, propylene glycol, and dextrose. Each possibility represents a separate embodiment. Suitable wetting agents include, but are not limited to, glycerin, cetyl alcohol, and glycerol monostearate. Each possibility represents a separate embodiment. Suitable preservatives include, but are not limited to, benzalkonium chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol, chlorohexidine, and polyhexamethylene biguanide. Each possibility represents a separate embodiment.

Suitable antioxidants include, but are not limited to, sorbic acid, ascorbic acid, ascorbate, glycine, α-tocopherol, butylated hydroxyanisole (BHA), and butylated hydroxytoluene (BHT). Each possibility represents a separate embodiment. Suitable flavoring agents include, but are not limited to, sweeteners such as sucralose and synthetic flavor oils and flavoring aromatics, natural oils, extracts from plants, leaves, flowers, and fruits, and combinations thereof. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot.

Each possibility represents a separate embodiment. Suitable colorants include, but are not limited to, alumina (dried aluminum hydroxide), annatto extract, calcium carbonate, canthaxanthin, caramel, β-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide. Each possibility represents a separate embodiment.

In certain aspects and embodiment, the pharmaceutical composition of the present invention is formulated as tablet, pill, capsule (e.g. soft or hard gelatin capsule), pellets, granules, powder, a wafer, coated or uncoated beads, lozenge, sachet, cachet, elixir, an osmotic pump, a depot system, an iontophoretic system, a patch, suspension, dispersion, emulsion, solution, syrup, aerosol, oil, ointment, suppository, a gel, and a cream. Each possibility represents a separate embodiment.

For preparing solid compositions such as tablets, the active pharmaceutical ingredient is mixed with a pharmaceutical carrier or excipient to form a solid pre-formulation composition containing a substantially homogeneous distribution of the compound of the present invention in the pharmaceutical carrier or excipient.

Any method can be used to prepare the pharmaceutical compositions. For example, solid dosage forms can be prepared by wet granulation, dry granulation, direct compression and the like as is known in the art. The liquid forms in which the compositions of the present invention may be incorporated, for administration via oral administration or by injection or another parenteral route, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles. Each possibility represents a separate embodiment.

In some particular embodiments, the composition comprises at least one of Compound 1a, Compound 1b and salts thereof. In another embodiment, said composition is for use as a medicament. In another embodiment said composition is for use in treating or preventing the progression of diabetes in a human subject in need thereof. In another embodiment said composition is for use in preserving or promoting the viability of pancreatic β cells. In another embodiment, said composition is for use in treating a subject diagnosed with a disease or condition as disclosed herein.

### Therapeutic Methods

In one aspect, there is provided a method for treating or preventing the progression of diabetes in a subject in need thereof, comprising administering to the subject a pharmaceutical composition comprising a compound of the invention.

The term “treating” as used herein refers to reduction, amelioration, inhibition and/or remission of a condition (e.g. diabetes) or a symptom or manifestation thereof. Symptoms characteristic of diabetes include, for example, elevated blood glucose levels, elevated glycosylated hemoglobin (HbA1C), decreased insulin production, insulin resistance, proteinuria, and impaired glomerular clearance. Preventing the progression (also referred to herein as inhibiting the progression) of diabetes refers in particular to reducing or delaying further development of the disease, for example from an early stage to a more advanced stage manifested by aggravated symptoms. In some embodiments, preventing the progression of diabetes includes preventing deterioration of the clinical state of the subject due to loss of β cell mass and/or activity. Diabetes mellitus (also referred to herein as “diabetes”) is a disease manifested by impaired insulin secretion and variable degrees of peripheral insulin resistance leading to hyperglycemia. Early symptoms are related to hyperglycemia and include polydipsia, polyphagia, polyuria, and blurred vision. Later complications include vascular disease, peripheral neuropathy, nephropathy, and predisposition to infection. Diagnostic criteria for diabetes according to the American Diabetes Association include fasting plasma glucose (FPG)≥7.0 mmol/L, oral glucose tolerance test, 2-hour glucose level (OGTT)≥11.1 mmol/L, and HbA1C≥6.5%. Impaired glucose regulation (impaired glucose tolerance, or impaired fasting glucose, also referred to herein as “pre-diabetes”) is an intermediate, possibly transitional, state between normal glucose metabolism and diabetes that becomes more common with aging, and is characterized by FPG of 5.6-6.9 mmol/L, OGTT of 7.8-11.0, and HbA1C of 5.7-6.4.

In one embodiment, the compound is represented by Formula I, or a salt thereof. In another embodiment, said compound is represented by Formula I-1, or a salt thereof. In other embodiments, said compound is selected from the group consisting of Compound 1a, Compound 1b, Compound 1c and salts thereof. In another embodiment, said compound is represented by Formula IIa, an ester or a salt thereof. In another embodiment, said compound is represented by Formula IIb, an ester or a salt thereof. In other embodiments, said compound is selected from the group consisting of Compound 2a, Compound 2b, Compound 2c esters and salts thereof. In another embodiment said compound is Compound 3, or an ester or a salt thereof. In another embodiment said compound is an EP3 antagonist. In another embodiment said compound inhibits palmitate-induced apoptosis in human islet β cells.

Typically, the subject to be treated by the methods of the invention is human. In another embodiment the diabetes is T2DM. In another embodiment the diabetes is T1DM. In another embodiment the diabetes is characterized by EP3 overexpression in pancreatic β cells. In a particular embodiment, the diabetes is characterized by EP₃-2 overexpression in pancreatic β cells. In another embodiment said subject is diagnosed with chronic hyperglycemia and dyslipidemia. In another embodiment said subject is pre-diabetic. In another embodiment said subject is obese. In another embodiment, said subject is a non-obese human subject. Each possibility represents a separate embodiment of the invention.

As used herein, the term “pre-diabetes” refers to a disease or condition that is generally characterized by impaired glucose tolerance and which frequently precedes the onset of diabetes in a subject. A pre-diabetic subject is typically identified as a subject characterized by a fasting blood glucose level greater than 100 mg/dl but less than or equal to 125 mg/dl, a 2-hour post-load glucose reading of greater than 140 mg/dl but less than 200 mg/dl, or a HbA1c level greater than or equal to 6.0% but less than 6.5%.

As used herein, the term “hyperglycemia” refers to elevated blood glucose levels in the body, which results from metabolic defects in production and utilization of glucose. A subject is identified as hyperglycemic if the subject has a fasting blood glucose level that consistently exceeds 126 mg/dl (e.g. for a duration of several months, reflected corresponding elevation in HbA1C levels.

With the term “dyslipidemia” a disorder of lipoprotein metabolism, including lipoprotein overproduction or deficiency is defined. Dyslipidemias (which encompasses and may further be referred to in the context of the invention as “hyperlipidemia”) may be manifested by elevation of the total cholesterol, the low-density lipoprotein (LDL) cholesterol and the triglyceride concentrations, and a decrease in the “good” high-density lipoprotein (HDL) cholesterol concentration in the blood. Dyslipidemia/hyperlipidemia within the meaning of the present invention is indicated when LDL cholesterol levels for adults more than 100 mg/dL (2.60 mmol/L), HDL cholesterol levels are equal to or lower than 40 mg/dL (1.02 mmol/L), and triglyceride levels are more than 150 mg/dL (1.7 mmol/L).

In the context of the present invention, unless otherwise specified, the term “obese” includes subjects that are classified in Obese Class I and above according to the World Health Organization (WHO) classification system. Thus, in embodiment the method is performed on a non-obese subject that has a BMI of less than 30 kg/m².

In another embodiment the composition is administered orally. In some embodiments, administration is performed in an amount and under condition sufficient to exert a beneficial therapeutic effect as disclosed herein. In various embodiments, the administration is performed so as to prevent or inhibit loss of pancreatic β cell mass and/or activity in said subject, to prevent or reduce the level or incidence of hyperglycemia in said subject, to prevent or reduce lipotoxicity-induced β cell damage, and/or to enhance glucose-induced insulin secretion by pancreatic β cells in said subject. Each possibility represents a separate embodiment of the invention. According to exemplary embodiments, compounds of the invention may be administered at therapeutically effective amounts of 0.01-100 mg/kg, e.g. at doses of 0.5-1, 0.1-10. 0.01-1 or 10-100 mg/kg for oral administration.

In one embodiment, the beneficial effect comprises prevention or inhibition of β cell mass, β cell activity, or both, wherein Each possibility represents a separate embodiment of the invention. By the term “pancreatic β-cell mass” is meant the total number of viable insulin-secreting pancreatic β-cells in a mammal (e.g., a human). The pancreatic β-cell mass may represent the total number of endogenous viable pancreatic β-cells in a subject or may represent the sum of the number of endogenous viable pancreatic β-cells in a subject plus the number of viable pancreatic β-cells transplanted into the subject (e.g., autograft, homograft, or xenografted viable pancreatic β-cells).

By the term “pancreatic β-cell function/activity” is meant a biological activity that is used to describe a mammalian (e.g., human) pancreatic β-cell (e.g., an activity that is specifically unique to a pancreatic β-cell). Non-limiting examples of pancreatic β-cell function include the synthesis and secretion of insulin, the synthesis and secretion of islet amyloid polypeptide (IAPP), and the synthesis and section of C-peptide. Methods for detecting the synthesis and secretion of insulin, IAPP, and C-peptide are known in the art and/or are exemplified herein. In particular, β cell function or activity may conveniently be evaluated in vitro by GSIS and in vivo by an oral glucose tolerance test (OGTT) as detailed herein, or using assays exemplified in the Examples section.

Methods of determining or assessing the pancreatic β-cell mass in a subject are known in the art, and include for example histology and/or imaging-based methods. Conveniently, molecular imaging modalities further providing metabolic and functional information, including, but not limited to positron emission tomography (PET) and single photon emission computed tomography (SPECT), may advantageously be used. For example, without limitation, Glucagon-Like Peptide-1 Receptor (GLP-1)-targeted imaging using exendin peptides (e.g. ¹¹¹indium-labeled exendin-4 derivatives), may conveniently be used in PET and/or SPECT imaging. In addition, alterations in pancreatic cell mass and/or activity may also be evaluated indirectly by various functional assays including, but not limited to determining blood glucose levels and determining HbA1C levels by methods known in the art.

In another embodiment, the beneficial effect comprises prevention or reduction of the incidence of hyperglycemia, manifested by a significant decrease in the rate of occurrence of hyperglycemic episodes in said subject compared to their rate prior to treatment. In another embodiment the beneficial effect comprises reducing the level of hyperglycemia, measurable by reduced blood glucose level or HbA1C in said subject.

In another embodiment, the beneficial effect comprises enhancement of glucose-induced insulin secretion (also referred to as GSIS) by pancreatic β cells in said subject. Assays for evaluating GSIS in vivo are known in the art and include, for example, ELISA assays as disclosed herein. In yet another embodiment, the beneficial effect comprises prevention or reduction of lipotoxicity-induced β cell damage. Lipotoxicity refers to the detrimental effects of lipid metabolites on nonadipose tissues, such as liver, skeletal muscle, heart, kidney, and pancreatic β cells. It is a pathologic process seen in various metabolic disorders, including T2DM. As used herein, “lipotoxicity” refers to any event that results in cellular damage that can range from mild cellular dysfunction to cell death. In various embodiments, β cell damage mediated by lipotoxicity may result in impairment in β cell function as disclosed herein and/or may result in β cell apoptosis, wherein each possibility represents a separate embodiment of the invention.

In another aspect, there is provided a method for preserving or promoting the viability of pancreatic β cells, comprising contacting a population (or preparation) of the β cells with a pharmaceutical composition comprising a compound of the invention, thereby preserving or promoting the viability of said β cells. As used herein, preserving or promoting β cell viability refers to preventing or inhibiting β cell death (either in culture or in vivo). In one embodiment, preserving or promoting β cell viability comprises inhibition of apoptosis (e.g. mediated by FFA).

According to particular embodiments, preserving or promoting β cell viability further refers to retaining β cell viability at a level representative of a healthy human subject. For example, viability may be preserved or maintained at a level at least 70%, 80%, 90%, 95%, 98% and up to 100% of the viability of β cells in a healthy control subject, wherein each possibility represents a separate embodiment of the invention.

In one embodiment, the compound is represented by Formula I, or a salt thereof. In another embodiment, said compound is represented by Formula I-1, or a salt thereof. In other embodiments, said compound is selected from the group consisting of Compound 1a, Compound 1b, Compound 1c and salts thereof. In another embodiment, said compound is represented by Formula IIa, an ester or a salt thereof. In another embodiment, said compound is represented by Formula IIb, an ester or a salt thereof. In other embodiments, said compound is selected from the group consisting of Compound 2a, Compound 2b, Compound 2c esters and salts thereof. In another embodiment said compound is Compound 3, or an ester or a salt thereof. In another embodiment said compound is an EP3 antagonist. In another embodiment said compound inhibits palmitate-induced apoptosis in human islet β cells.

In one embodiment, the contacting is performed in vivo. In another embodiment the contacting is performed ex vivo. In another embodiment the contacting is performed in vitro.

In another embodiment said β cells are characterized by EP3 (e.g. EP₃-2) overexpression (e.g. by at least 2-fold and typically 2.5-10-fold enhancement in the EP3 or EP₃-2 transcript levels). In some embodiments, the β cells or islets are obtained from a human subject having a pancreatic β cell disorder selected from the group consisting of: pancreatic β cell failure (characterized by loss of the GSIS ability of said cells), T2DM, T1DM, pre-diabetes and insulin resistance, or from a subject diagnosed with chronic hyperglycemia and dyslipidemia, wherein each possibility represents a separate embodiment of the invention. In another embodiment, said β cells are obtained from a healthy human donor. In various other embodiments, the cell population (or preparation) is selected from the group consisting of purified primary β cells, β cell lines, genetically modified β cells, primary pancreatic islet cells, and fetal pancreatic islet cells, wherein each possibility represents a separate embodiment of the invention.

In another embodiment the method comprises administering the composition to a human subject having a pancreatic β cell disorder selected from the group consisting of: pancreatic β cell failure, T2DM, T1DM, pre-diabetes and insulin resistance, or from a subject diagnosed with chronic hyperglycemia and dyslipidemia. In another embodiment said composition is administered orally.

In another embodiment said subject further receives a pancreatic β cell transplantation, a pancreatic islet transplantation or a pancreatic transplantation.

In another embodiment said contacting is performed at a concentration of 250 nM-200 μM of said compound (e.g. 0.25-10, 0.5-5 or 5-10 μM) for 24-48 hours. In another embodiment said contacting is performed so as to prevent or reduce lipotoxicity-induced β cell damage. In another embodiment said contacting is performed so as to exert a beneficial EP3-mediated biological activity as disclosed herein.

In another embodiment there is provided a cell composition for pancreatic β cell transplantation or pancreatic islet transplantation, comprising the β cell population (or preparation) that has been contacted with said pharmaceutical composition as disclosed herein.

In another aspect, the invention provides a method of preventing lipotoxicity-induced β cell damage in a subject diagnosed with T2DM, comprising contacting pancreatic β cells of the subject with a pharmaceutical composition comprising a compound of the invention.

In some embodiments, the compound is represented by Formula I, or a salt thereof. In another embodiment, said compound is represented by Formula I-1, or a salt thereof. In other embodiments, said compound is selected from the group consisting of Compound 1a, Compound 1b, Compound 1c and salts thereof. In another embodiment, said compound is represented by Formula IIa, an ester or a salt thereof. In another embodiment, said compound is represented by Formula IIb, an ester or a salt thereof. In other embodiments, said compound is selected from the group consisting of Compound 2a, Compound 2b, Compound 2c esters and salts thereof. In another embodiment said compound is Compound 3, or an ester or a salt thereof.

According to some embodiments, the methods of the invention may be used for prediabetic patients (impaired glucose tolerance, IGT) and newly diagnosed T2DM patients. It is understood by the skilled the art that β-cell apoptosis is an incremental process in which the β-cell population is gradually decreasing. It may therefore be advantageous to make use of the method in patients newly diagnosed with diabetes (e.g. T2DM) or diagnosed with a pre-stage of T2DM or prediabetes since the β-cell population is still largely present but functionally impaired (e.g. reduced GSIS) and the benefit of rescuing from β-cell loss of function and apoptosis most prevalent. Additionally, or alternatively, the method may be used for any patient afflicted with, or at risk of developing, diabetes (e.g. T2DM or T1DM). In other embodiments, the methods of the invention may be used in the treatment of advanced stage diabetes patients, as long as a population of pancreatic β cells is still present. In some embodiments, since the compounds of the invention may be administered orally, they provide for enhanced compliance thereby maximizing efficacy, and for enhanced safety compared to existing treatments such as GLP-1 antagonists. In other embodiments, the subject to be treated by the methods of the invention is a non-obese human subject. In other embodiments, the subject is not concomitantly afflicted with cancer or an infection. In yet other embodiments, said subject is not afflicted with an eating disorder, dyslipidemia, T2DM or a cardiovascular disease. Each possibility represents a separate embodiment of the invention.

### Pancreatic Cell Populations and Compositions for Transplantation

The invention further refers in embodiments thereof to cell compositions, including composition for pancreatic β cell transplantation and pancreatic islet transplantation. As disclosed herein, cell compositions in accordance with the invention comprise β cell preparations that have been contacted with one or more compounds of the invention, as disclosed herein. In other embodiments, a subject to be treated by the compounds of the invention further receives a pancreatic β cell transplantation, a pancreatic islet transplantation or a pancreatic transplantation. Each possibility represents a separate embodiment of the invention.

The tissues with an endocrine role within the pancreas exist as clusters of cells called pancreatic islets (also called islets of Langerhans) that are distributed throughout the pancreas. Pancreatic islets contain a cells, β cells and δ cells, each of which releases a different hormone. These cells have characteristic positions, with α cells (secreting glucagon) tending to be situated around the periphery of the islet, and β cells (secreting insulin) more numerous and found throughout the islet. Enterochromaffin cells are also scattered throughout the islets. Islets are composed of up to 3,000 secretory cells, and contain several small arterioles to receive blood, and venules that allow the hormones secreted by the cells to enter the systemic circulation.

In some embodiments, a pancreatic transplantation is used, e.g. according to one of four common procedures including pancreas transplant alone (PTA), simultaneous pancreas-kidney transplant (SPK, pancreas-after-kidney transplant (PAK), and simultaneous deceased donor pancreas and live donor kidney (SPLK) transplant. In another embodiment, the invention pertains to compositions and methods for use in connection with pancreatic islet transplantation protocols.

An “islet”, “pancreatic islet” or “islet of Langerhans” includes populations of cells which produce hormones in response to glucose levels including 0-cells. In certain embodiments, an islet may include, without limitation, an islet of a subject, a human cadaver islet, an islet to be transplanted into a subject, or an islet transplanted into a subject. For example, one islet equivalent (IE) is an islet of diameter 150 μm, typically containing 1,500-2,000 cells, including 40-60% β cells.

For example, the Edmonton protocol may be applied for pancreatic islet transplantation, wherein specialized enzymes (e.g. LIBERASE human islet enzyme, Roche Diagnostics) are used to remove islets from the pancreas of a deceased donor. Cells are then purified by centrifugation and/or apheresis (e.g. on continuous Ficoll gradients on a cooled apheresis system) and re-suspended in a suitable medium (e.g. transplant medium, CMRL 1066, Mediatech). Typically, a patient receives at least 10,000 IE per kilogram of body weight, extracted from two donor pancreases. Patients often require two transplants to achieve insulin independence. Transplants are often performed by a radiologist, who uses x rays and ultrasound to guide placement of a catheter through the upper abdomen and into the portal vein of the liver. The islets are then infused slowly through the catheter into the liver. In some cases, a surgeon may perform the transplant through a small incision, using general anesthesia. Various protocols for pancreatic islet transplantation for treating insulin-related disorders and other conditions are described, for example, by Rickels et al. (Endocr Rev. 2019; 40(2):631-668), incorporated herein by reference.

Although islet transplantation was demonstrated as an effective treatment for T1DM, long-term therapeutic benefit of currently used protocols remains challenging. Upon transplantation, ˜60% of transplanted islets are destroyed by the instant blood-mediated inflammatory response, leaving a marginal β cell mass that is susceptible to metabolic exhaustion, hypoxic damage, and immune rejection. To prevent rejection and recurrence of autoimmunity, potent systemic immunosuppression with a combination of T cell ablation, co-stimulation blockade, calcineurin inhibitors, and mammalian target of rapamycin inhibitors is typically required. However, chronic immunosuppressive therapy is associated with life-threatening adverse effects such as opportunistic infection, organ toxicity, and neoplasm development. Without wishing to be bound by a specific theory or mechanism of action, the compositions and methods of the invention provide for β cell replacement therapeutic modalities (inter alia islet transplantation and β cell transplantation) providing for reducing the number of initial cells required for transplantation. For example, without limitation, the invention in embodiments thereof pertains to cell compositions wherein the IE may be reduced by e.g. 5, 10, 20, 30, 40, 50% or more compared to a conventional islet composition. According to exemplary embodiments, a cell composition of the invention may contain 700,000, 600,000, 500,000, 400,000, 300,000, 200,000 or 150,000 IE (or an equivalent dose of β cells for β cell compositions as detailed below). In other exemplary embodiments, the composition may contain about 5000 IE/kg.

In another embodiment, the invention pertains to compositions and methods for use in connection with β cell transplantation protocols.

By the term “pancreatic β-cell”, “β cell” or “islet β cell” is meant an insulin-producing cell corresponding to a β cell normally present in (or obtained from) the pancreas of a mammal in the islet of Langerhans, as described herein. As used herein, the term pancreatic β-cell encompasses a pancreatic β-cell present in the body of a mammal (e.g., endogenous pancreatic β-cells, or autograft, homograft, or xenograft pancreatic β-cells) or a pancreatic β-cell cultured in vitro (for example an ex vivo (e.g., primary) culture of pancreatic β-cells from any mammalian species described herein or a pancreatic β-cell line (e.g., a primary or immortalized cell line). In some embodiments, the pancreatic β-cell present in a mammal is present in the pancreas. In some embodiments, the pancreatic β-cell present in a mammal is located in a tissue other than the pancreas (e.g., in liver tissue following transplantation). In some embodiments, the pancreatic 3-cell can be genetically manipulated using molecular biology techniques to express one or more recombinant proteins (e.g., an insulin) and/or decrease the expression of one or more endogenous proteins.

By the phrase “population of cells” (e.g. pancreatic β-cells) is meant a plurality of said cells (e.g. an endogenous cell population of a subject or an experimentally generated cell population). A preparation of β cells as used herein refers to an in vitro or ex vivo generated β cell population (e.g. purified from pancreatic islets, differentiated from pluripotent stem cells, or cultured β cell lines). In some embodiments, the term preparation refers in particular to purified or substantially purified cell populations (substantially free of additional cell types). In additional embodiments, a cell preparation for transplantation (such as the cell compositions as disclosed herein) comprises a therapeutically effective amount of said cells, as further described herein. For example, without limitation, cell preparations to be used in embodiments of the invention may be e.g. purified primary β cells, β cell lines, genetically modified β cells, primary pancreatic islet cells, and fetal pancreatic islet cells, wherein each possibility represents a separate embodiment of the invention.

In another embodiment, β cells or islets to be used in transplantation may be obtained from a healthy human donor (a non-diabetic subject having normal β cell function, including a deceased donor with functional pancreatic islets). In some embodiments, the transplanted pancreatic β-cells are autografted, homografted, or xenografted. In some embodiments, the transplanted pancreatic β-cells are present within a device, or are surrounded by or placed within a biocompatible polymer (microencapsulated or microencapsulated. In other embodiments, embryonic stem cells (ESCs, isolated from the early embryo), mesenchymal stem cells (MSCs), and iPSCs (reprogramed from somatic cells into embryonic-like pluripotent state) can be differentiated into insulin-producing cells for use in β cell transplantation protocols. Paez-Mayorga et al. (Trends in Pharmacological Sciences, March 2022, Vol. 43, No. 3) exemplifies approaches and protocols for β cell transplantation (including in encapsulated form).

In accordance with an embodiment of the present invention, an artificial islet or pancreas is provided. The artificial islet or pancreas can be constructed using the β cells generated according to the methods described herein, in an implantable device that encapsulates and nurtures these cells. An artificial pancreas may contain a million islets or more, and may be implanted in the peritoneal cavity or under the skin where it can respond to changing blood glucose levels by releasing hormones, such as insulin. An artificial pancreas may be made using living (e.g., glucose-sensing and insulin secreting islets) and nonliving components (e.g., to shield the islets from the diabetic's body and its destructive immune mechanism while permitting the islets to thrive).

In some aspects, the artificial pancreas comprises a macroencapsulation device into which islet cells comprising β cells generated according to the methods herein are grouped together and encapsulated. In some aspects, the macroencapsulation device comprises a PVA hydrogel sheet for an artificial pancreas of the present invention. In some aspects, the artificial islet comprises β cells generated according to the methods herein, along with other islet cells (a, 6, etc.) in the form of an islet sheet. The islet sheet comprises a layer of artificial human islets comprising the β cells macroencapsulated within a membrane (e.g., of ultra-pure alginate). The sheet membrane is reinforced with mesh and may be coated on the surface to prevent or minimize contact between the cells encapsulated inside and the transplantation recipient's host immune response. Oxygen, glucose, and other nutrients readily diffuse into the sheet through the membrane nurturing the islets, and hormones, such as insulin readily diffuse out. Additional examples of membranes designed for macroencapsulation/implantation of an artificial islet or pancreas can be found in the literature (e.g. Paez-Mayorga et al., ibid).

In some embodiments, there is provided a cell composition for pancreatic β cell transplantation or pancreatic islet transplantation as described herein that has been contacted with a compound of the invention by a method as disclosed herein. For the manufacture of a cell composition for transplantation, an effective amount of the treated cells as disclosed herein is formulated with suitable clinical-grade excipients (e.g. 3,000-15,000 IE/kg suspended in GMP-grade transplant medium supplemented with human serum albumin, heparin, and ciprofloxacin). In some embodiments, the cell composition is used for treating or preventing the progression of diabetes in a human subject in need thereof. In other embodiments, said cell composition is used for treating a subject afflicted with a disease or condition as disclosed herein (e.g. T1DM or T2DM). In various embodiments, said cell composition is autologous, histocompatible allogeneic, non-histocompatible allogeneic, or xenogeneic to said subject, wherein each possibility represents a separate embodiment of the invention.

The following examples are presented in order to more fully illustrate some embodiments of the invention. They should, in no way be construed, however, as limiting the broad scope of the invention.

### Examples

### Materials and Methods

Reagents: Sodium palmitate (Sigma Aldrich Co.) was dissolved in 95% ethanol at 60° C. and added to the preheated medium. The EP3 antagonist L-798106 (Tocris, Ellisville, MO) was dissolved in DMSO. The designations of the compounds as used in the Examples below are as follows: C13—compound 1a; C5—compound 1b; C14—compound 1c; C17—Compound 2a; C15—Compound 2b; C20—Compound 2c; C12—Compound 3; C9—Compound 4b; C4 Compound 5b. C10 is the compound N-(4-chloro-2-(1H-pyrrole-2-carbonyl)phenyl)-1-ethyl-3,5-dimethyl-1H-pyrazole-4-sulfonamide.

Islet and cell culture: The pancreatic β-cell line MIN6, which is a clonal murine insulin-secreting line, was cultured in DMEM (Biological Industries, Beit Haemek, Israel) supplemented with 11 mM Glucose, 2 mM glutamine, 15% heat-inactivated fetal calf serum (FCS), 100 U/ml penicillin, 100 g/ml streptomycin and 61 μM β-mercaptoethanol. Human islets were obtained from the European Consortium for Islet Transplantation (ECIT) at the San Raffaele Hospital and from the Niguarda Hospital, (Milan, Italy) and cultured in CMRL-1066 media (Biological Industries, Beit HaEmek, Israel) supplemented with 10% FCS, 5.5 mM glucose, 2 mM L-Glutamine, 100 U/ml penicillin, 100 μg/ml streptomycin and 150 μg/ml gentamycin. Palmitate was added to 1% FCS and 0.5% defatted BSA (Roche, Germany).

Western blotting: Cells were lyzed in Laemmli protein sample buffer, separated on SDS-polyacrylamide gel, and transferred to 0.2 m nitrocellulose membranes (Schleicher and Schuell, Dassel, Germany). After blocking in 5% dried skimmed milk, the membranes were first incubated with the appropriate antibodies against: cleaved caspase-3 (Asp175) (Cell Signaling Technology Danvers, MA) and α-tubulin (Sigma-Aldrich.) and then with horseradish peroxidase conjugated anti-rabbit or anti-mouse IgG, followed by analysis with an enhanced chemiluminescence detection kit (Biological Industries, Israel).

Statistical analysis: Statistical significance for differences among groups was determined by use of one-way ANOVA and Student's t test to compare treated with untreated samples. All statistics were calculated using GraphPad software (La Jolla, CA, USA). p value <0.05 was considered.

Determination of glucose-stimulated insulin secretion (GSIS) and content: To determine acute insulin release in response to glucose stimulation, islets were washed with Krebs-Ringer bicarbonate buffer pH 7.4, containing 10 mM HEPES and 0.5% bovine serum albumin (KRBH), and pre-incubated for 2 hrs in the same buffer containing 3.3 mM glucose. The buffer was then discarded and replaced with fresh KRBH containing 3.3 mM glucose, and the islets incubated for 1 hour for basal insulin secretion, followed by additional 1 hour incubation in KRBH containing 16.7 mM glucose. The supernatants were collected, centrifuged at 900 rpm and insulin was determined by Rat or Human insulin radioimmunoassay (RIA) kits (Linco Research, St. Charles, MO) according to the Manufacturer's instructions. The islets or cells after the insulin secretion assay were washed with phosphate-buffered saline (PBS) and extracted in 70% ethanol containing 0.18 N HCl for 24 h at 4° C. for insulin content. The acidic ethanol eluates were collected and the amount of insulin determined by ELISA.

ELISA Assay: Insulin was extracted from the intracellular storage granules in human islets cells using a lysis buffer (Ethanol-HCl). The extract was analysed by a specific ELISA (Mercodia) which is a solid phase two-site enzyme immunoassay based on the sandwich technique, in which two monoclonal antibodies are directed against separate antigenic determinants on the insulin molecule. Insulin in the sample reacts with anti-insulin antibodies bound to microtitration wells and peroxidase-conjugated anti-insulin antibodies in the solution.

Real-time PCR: Total RNA from MIN6 cells or islets was extracted using TRIzol (Invitrogen, Carlsbad, CA, USA) and 2 μg of total RNA was converted to cDNA using SuperScript® III Reverse Transcriptase (Invitrogen, Carlsbad, CA, USA). 2.5-5 ng cDNA was used for each real-time PCR reaction. Real-time reaction was performed using Platinum SYBR Green qPCR SuperMix-UDG with ROX reference dye (Applied Biosystems). The primers used were as follows: GAPDH forward (F): CGTGTTCCTACCCCCAATGT; GAPDH reverse (R): TGCTTCACCACCTTCTTGATGT; Ins1 (F): AACCCCCAGCCCTTAGTGA; (R): GGGTAGGAAGTGCACCAACAG; Ins2 (F): ACCCACAAGTGGCACAACTG; (R): GATCTACAATGCCACGCTTCTG (SEQ ID Nos: 1-6, respectively). Additional primers used as controls are directed to HPRT, as follows: HPRT (F): TCCCATCTCCTTCATGACATCTC HPRT (R) GCCGAGGATTTGGAAAAAGTG (SEQ ID Nos: 7-8, respectively).

### Example 1. Screening and Identification of EP3 Antagonist Compounds

To identify candidate EP3 antagonist compounds, an In Silico multi-filter modeling “Iterative Stochastic Elimination” (ISE) was used to construct and build a model based on hundreds of molecules known for their high activity to the specific EP3 receptor and screen about 2,170,859 molecules from a library (EnAmine database).

To this end, four Iterative Stochastic Elimination (ISE) models (2-Homo Sapiens and 2—Mouse) were built, based on known EP3 Ki and IC50 values. The ISE algorithm enabled to classify the molecules activity through 2D descriptors. 21 common molecules were found between the human EP3 antagonist IC50-Model 1 and the mouse EP3 antagonist IC50 Model-3. There were 16 common molecules found between the Mouse EP3 antagonist IC50 Model-3 and the Mouse EP3 antagonist Ki Model-4. Between the human EP3 antagonist IC50 Model-1 and the human EP3 antagonist Ki model 2,437, common molecules were found.

The top 20 identified molecules out of the about 2.17 million screened molecules were selected for further examination. The compounds were initially tested for cytotoxicity against MIN6 pancreatic β cells. Compounds C6, C7 and C11 were found to be cytotoxic, and discarded from further use and compound C3 was discarded for poor water solubility. The compounds found to be non-cytotoxic at the tested concentration (10 μM) were further tested for efficacy and safety in various in vitro, ex vivo and in vivo models, as described hereinbelow.

### Example 2. Screening for Compounds Having Anti-Apoptotic Effect on Palmitate-Treated MIN6 Cells

The remaining compounds were screened for the ability to rescue β-cells from palmitate-induced apoptosis as compared to the commercial EP3 antagonist L-798106 by measuring the levels of the cleaved caspase 3 apoptotic marker, using Western Blot analysis (FIG. 1). To this end, MIN6 cells were treated with L-798106 or with the tested compounds (10 PM), in the presence or absence of palmitic acid (0.3 mM) for 48 hours. Cell extracts were analyzed by western blotting for the presence of cleaved caspase 3 as compared to α-Tubulin. The intensity of the bands was measured and the ratio of the average 4-6 independent experiments is presented in FIG. 1.

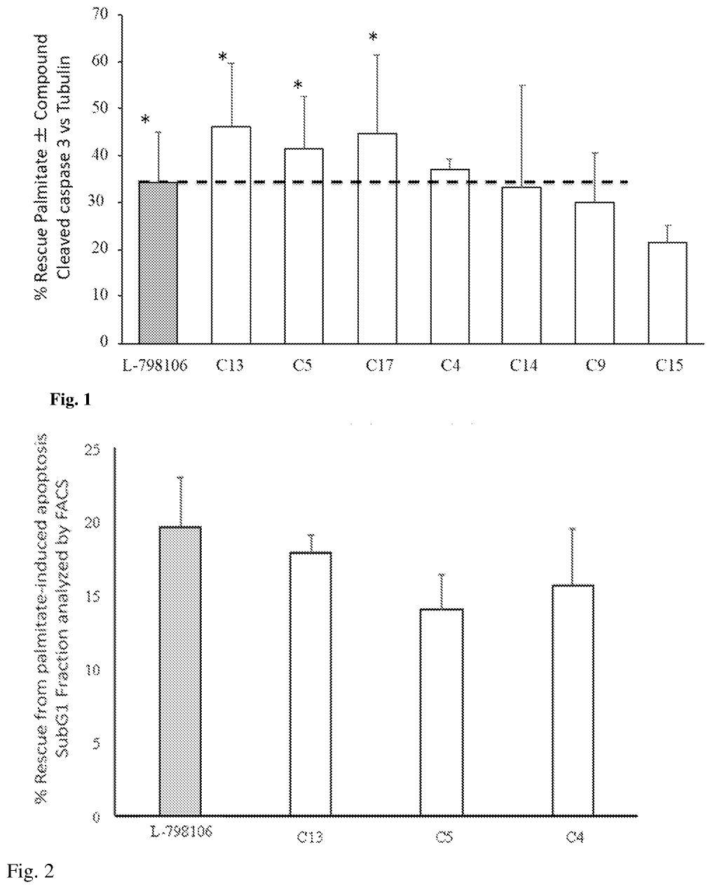

The ability of the compounds to rescue β-cells from apoptosis was also evaluated using FACS analysis and measuring the SubG1 population (FIG. 2). To this end, MIN6 cells were treated with L-798106 or with the tested compounds, in the presence or absence of palmitic acid for 48 hours. Collected cells were analyzed by FACS and the SubG1 fractions (representing apoptotic cell products or components were compared. The average of 4-6 independent experiments is presented in FIG. 2.

As can be determined from FIGS. 1 and 2, several compounds were able to rescue the cells from apoptosis at least to the same extent as the commercial EP3 antagonist L-798106 at a statistically significant manner. Notably, compounds C5, C13 and C17 exhibited exceptionally high efficacy in inhibiting or reversing palmitate-induced MIN6 cell apoptosis.

### Example 3. Screening for Compounds Having Anti-Apoptotic Effect on Palmitate-Treated Human Islet Cells

Next, the ability of the compounds to rescue human islet cells from palmitate-induced apoptosis was determined. To this end, the Caspase-Glo 3/7 assay reagent (Promega) was used for caspase 3/7 detection in palmitic acid-treated cells. The reagent provides a proluminescent caspase-3/7 substrate, which contains the tetrapeptide sequence DEVD, in combination with luciferase and a cell-lysing agent. The addition of the reagent directly to the wells results in cell lysis, followed by caspase cleavage of the DEVD substrate, and the generation of luminescence. The amount of luminescence (Relative Light Units, RLU) as displayed on the readout is proportional to the amount of caspase 3/7 activity in the sample.

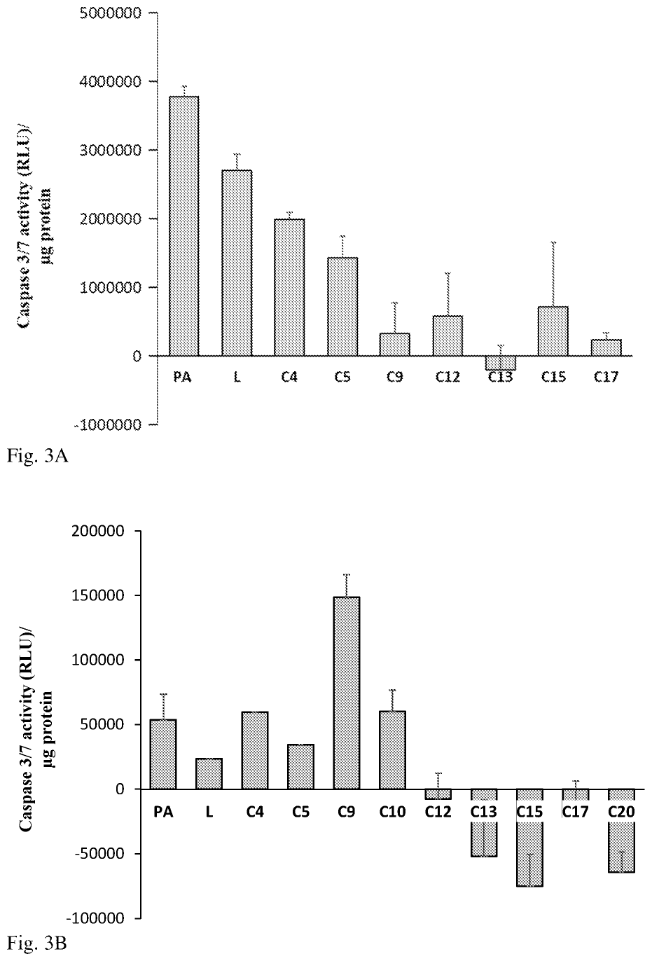

Accordingly, human islets from two donors were exposed to the various compounds or to L-798106 for 48 h in the presence of palmitic acid, and the bioluminescence was measured as compared to control cells. FIGS. 3A-3B show the results of the treatment with 5 M of the compounds in two human donors (FIG. 3A—donor 1, FIG. 3B—donor 2). PA: Palmitate; L: L-798106; RLU: Relative Light Units. The results are average of triplicates or quadruplicates ±SEM (except in islets from L-798106 treated donor 2, representing duplicates).

As can be seen in FIGS. 3A-3B, several compounds were identified, capable of rescuing human islet cells from apoptosis to a greater extent than the EP3 antagonist L-798106. Exemplary identified compounds are: C13, C5, C12, C15, C17 and C20. Notably, compound C13 exhibited particularly high efficacy in inhibiting or reversing palmitate-induced apoptosis in pancreatic islets of both human donors. It is further noted that in donor 1, compound C9 exhibited marked inhibitory activity, and compound C4 demonstrated moderate inhibitory activity comparable to that of L-798106 (FIG. 3A). In contrast, other compounds tested, such as C10, which was identified as a putative EP3 inhibitor in silico, failed to inhibit palmitate-induced apoptosis in pancreatic islets of both human donors.

### Example 4. Dose Response Experiments

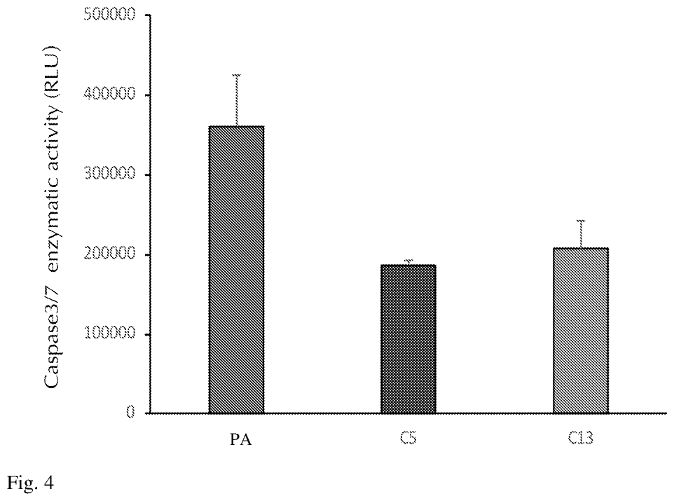

Following the initial screening results, compounds C13 and C5 were tested at different doses for their ability to reduce palmitate-induced apoptosis. To this end, MIN6 cells were exposed to 250 nM of the tested compounds in the presence of palmitic acid (PA) for 24 h, or to PA alone. Caspase 3/7 enzymatic activity was measured as described in Example 3, and the results, presented in FIG. 4 represent the average ±SEM of triplicates or quadruplicates. As can be seen in FIG. 4, both compounds significantly reduced palmitate-induced apoptosis in MIN6 cells, even at a concentration as low as 250 nM.

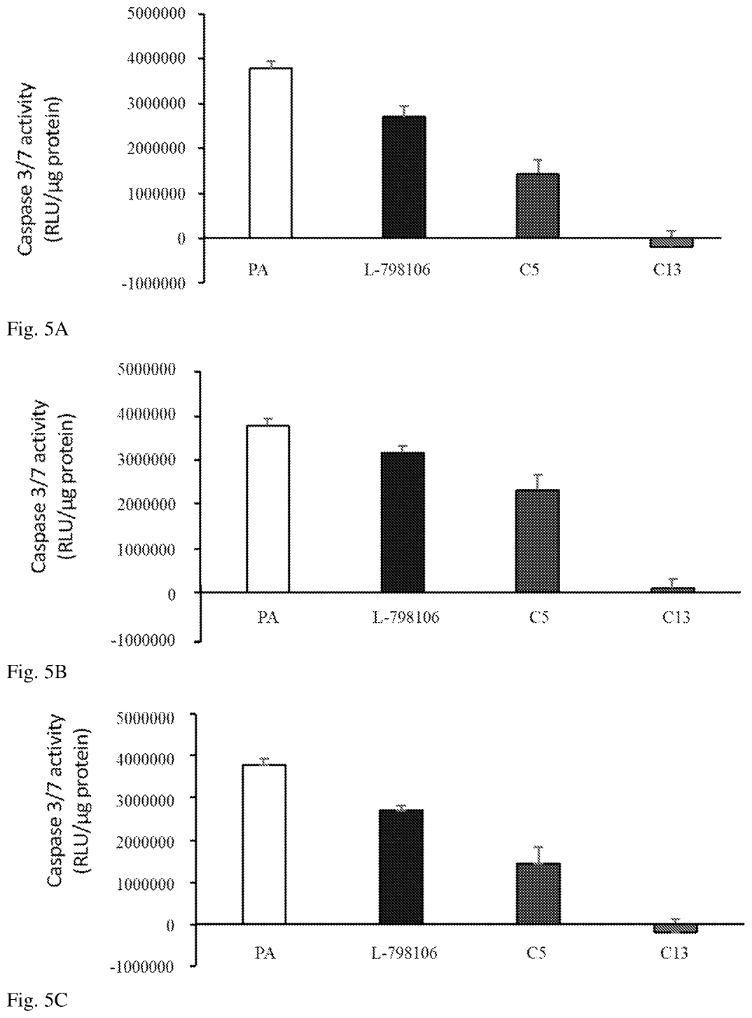

Next, various concentrations of the compounds were tested in human pancreatic islet cells. Cells obtained from the first healthy donor (donor 1) were incubated in the presence or absence of PA with C13, C5 or L-798106 (L) at concentrations of 5, 2.5 and 0.5 μM. Caspase 3/7 enzymatic activity was measured as described above. The enzymatic activity is presented in FIG. 5A for 5 μM, FIG. 5B for 2.5 μM and FIG. 5C for 0.5 μM as relative light units (RLU/μg protein) and the graphs represent the average of n=3-4. As can be seen in FIGS. 5A-5C, the ability of compounds C5 and C13 to inhibit palmitate-induced apoptosis in MIN6 cells to a greater extent than L-798106 was maintained in human islets even at the low concentrations. As can further be seen, C13 exhibited remarkable efficacy at all tested concentrations.

### Example 5. Effect on Palmitate-Induced Downregulation of Insulin Gene Expression

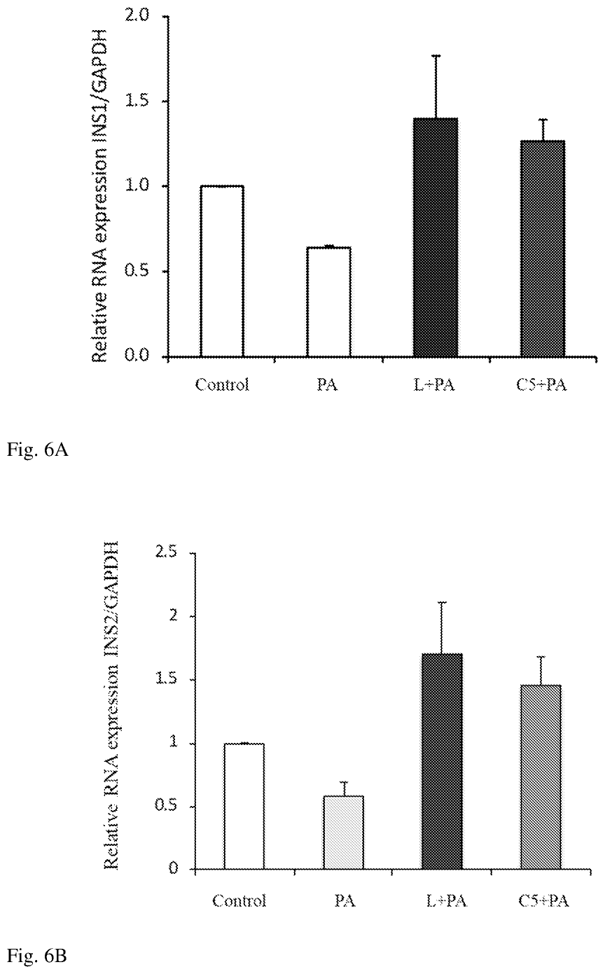

PA decreases the expression of β-cell specific genes, including insulin. The effect of the selected compounds on insulin gene expression was further tested in MIN6 cells. Rodent β-cells have two insulin genes (INS1 and INS2). MIN6 cells were exposed to C5 or L-798106 (10 μM) in the presence or absence of palmitic acid, and mRNAs insulin levels were measured by real-time PCR. FIGS. 6A-6B present the expression levels of INS1 and INS2 genes, respectively, normalized to GAPDH expression levels. Data of n=3±SEM is presented. As can be seen in FIGS. 6A-6B, both compounds enhanced insulin gene expression despite the presence of palmitate, wherein the effect of C5 appeared to be more significant.

### Example 6. Effect on Insulin Granules in β Cells

Palmitic acid further results in decreased insulin content in insulin storage vesicles in β-cells, and in impaired glucose-stimulated insulin secretion (GSIS). The effect of the compounds on insulin storage and secretion was further examined.

To this end, batches of human islets were pre-incubated with palmitic acid (0.3 mM) and 5 M of the different test compounds (L-798106, C5, or C13) for 48 hr. Islets were then incubated sequentially in culture media containing altered glucose concentrations: 3.3 mM glucose-containing medium to examine basal insulin secretion, followed by 16.7 mM glucose-containing medium to examine glucose-induced insulin secretion. The levels of insulin secreted to the media, and the levels retaining in the cells, were determined by ELISA.

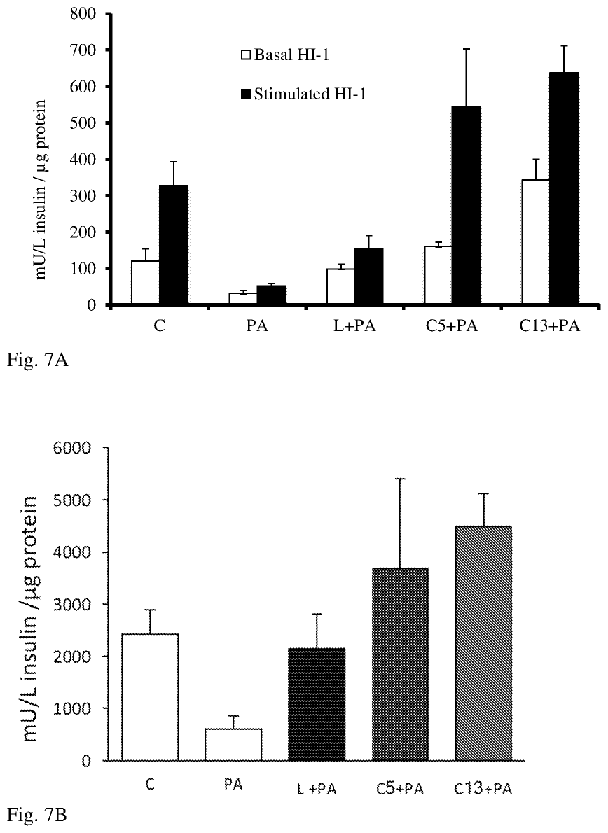

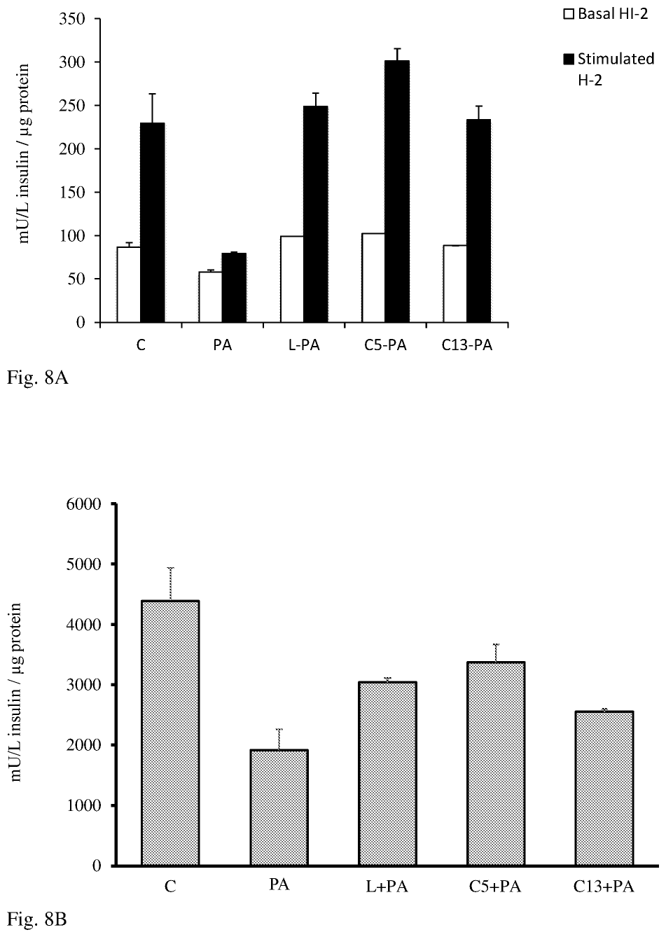

The results obtained in islets of donor 1 and donor 2 are presented in FIGS. 7A-7B and 8A-8B, respectively, in which: C represents control cells, without pre-incubation with palmitic acid or the test compounds, PA represents palmitate-treated cells, L represents L-798106, HI-1 represents donor 1 human islets, and HI-2 represents donor 2 human islets. FIGS. 7A and 8A show basal insulin secretion (Basal) and glucose-stimulated insulin secretion (Stimulated), and FIGS. 7B and 8B show insulin content remaining in the treated cells. The data represents the average ±SEM of n=4 for donor 1 and the average ±SEM of n=3 for donor 2.

As can be seen in FIGS. 7A-B and 8A-B, the test compounds and in particular C13 were more potent than L-798106 in restoring glucose-stimulated insulin secretion and content in human islets β-cells treated with palmitate.

### Example 7. In Vivo Toxicity

The safety of compounds C5 and C13 was examined in various in vivo settings, and compared to that of L-798106. The compounds were administered by oral gavage or intraperitoneal (IP) injection to C57Bl/6 mice, and acute toxicity, liver and kidney functions and blood glucose and insulin levels were evaluated. Additional safety experiments and measurements of various blood parameters in diabetic mice are further presented in Example 12 below.

Acute toxicity—IP and oral. For acute toxicity studies, a single IP dose of 3 mg/kg, or three oral doses (total of 9 mg/Kg) were administered. No mortality was observed by the end of the monitoring period (two days for IP administration or four days for oral administration), and all animals survived both modes of administration. No physical or behavioral differences were observed between the different groups and between the two modes of drug administration. Pictures of the sites of IP injection were taken when animals were sacrificed and no damage was observed.

### Oral Administration.

Male mice (n=8 per group) were assigned to the following test groups. A dosage of 3 mg/kg/mouse was administered daily for 3 consecutive days in 200 μl of:

Group A: saline and DMSO carrier (referred to throughout the drawings and examples describing the in vivo experiment as “Control-Saline”, “Vehicle”, “Saline” or “Control”), at the equivalent concentration administered the other test groups (2.5%). Group B: Compound 1b (C5) at 3 mg/Kg: (total of 9 mg/Kg). Group C: Compound 1a (C13) at 3 mg/Kg: (total of 9 mg/Kg). Group D: L-798106 at 3 mg/Kg: (total of 9 mg/Kg).

A. For glycemia evaluation, blood glucose was measured from the vein tail at the following time points (t(h)—time in hours): t=0—before gavage; t=5 h—following each gavage on days 1, 2 and 3 (5G1, 5G2 and 5G3, respectively); and t=24 h—after the last gavage (24G3).

Blood was collected by cardiac puncture, and 50 μl were collected in EDTA containing tubes for cell blood count (CBC). Further, 200-300 μl of blood was centrifuged and serum was collected for biochemical analysis.

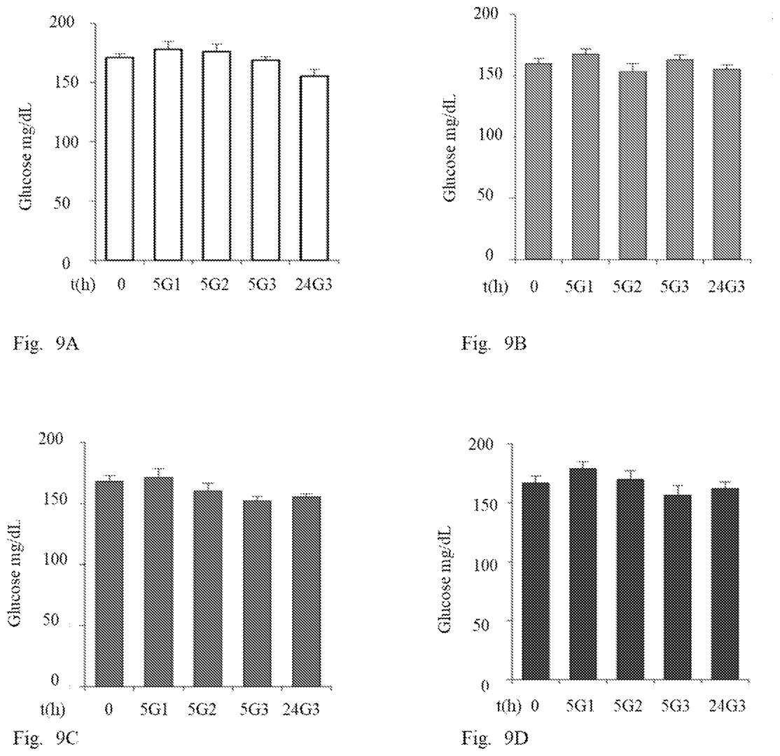

Values (±SEM) of glucose concentrations are presented in FIG. 9A for the control (Saline), FIG. 9B for C5, FIG. 9C for C13, and FIG. 9D for L-798106. The statistical analyses were performed using one-way Anova. As can be seen in FIG. 9A-9D, no significant alterations in either of the treatment groups or between the different groups were observed, and all the p-values >0.05.

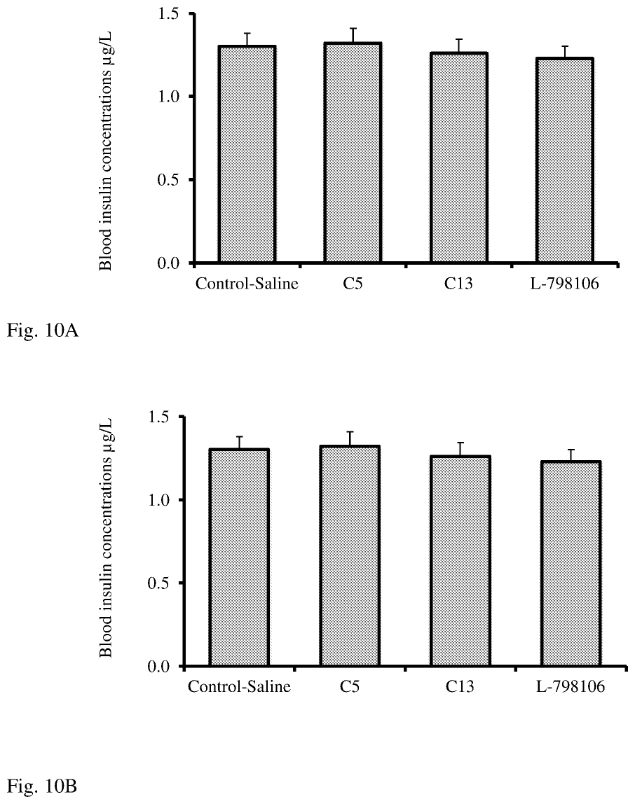

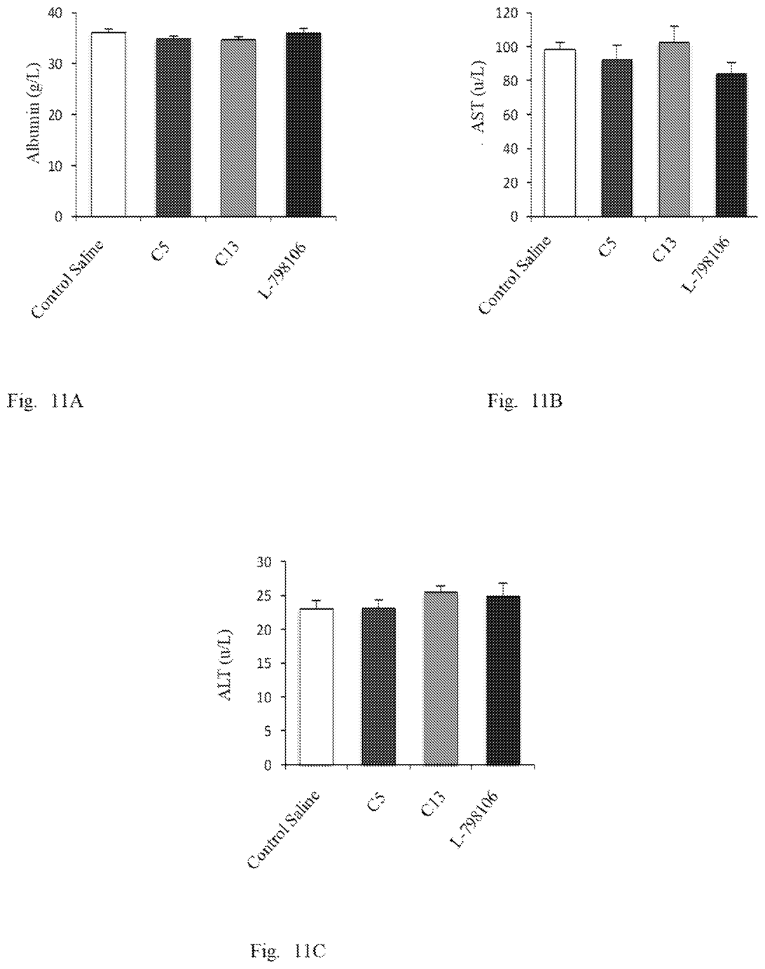

B. For measurements of blood insulin levels, insulin was measured in blood obtained from the vein tail of C57Bl/6 mice (n=8/group) 24 h (t24) after the third gavage with vehicle (Control-Saline); 3 mg/kg/day Compound C5; Compound C13 or the EP3 antagonist L-798106 (total 9 mg/kg) using ELISA mouse insulin kit (Mercodia). Five samples randomly chosen from each group were measured. As can be seen in FIG. 10A, no statistically significant differences were observed between the two experimental groups compared to the control.

C. Effects of orally administered compounds on liver function parameters: The blood levels of albumin, the liver aspartate transaminase (AST) and alanine transaminase (ALT) were measured in the sera obtained from mice 24 h after the third gavage with saline/DMSO (Control Saline); 3 mg/kg/day Compound C5; Compound C13 or the EP3 antagonist L-798106. Results are presented in FIG. 11A for Albumin, FIG. 11B for AST, and FIG. 11C for ALT. As can be seen in FIGS. 11A-11C, the results show no statistically significant differences for each test between the two experimental groups and the control (saline) group. All p-values >0.05.

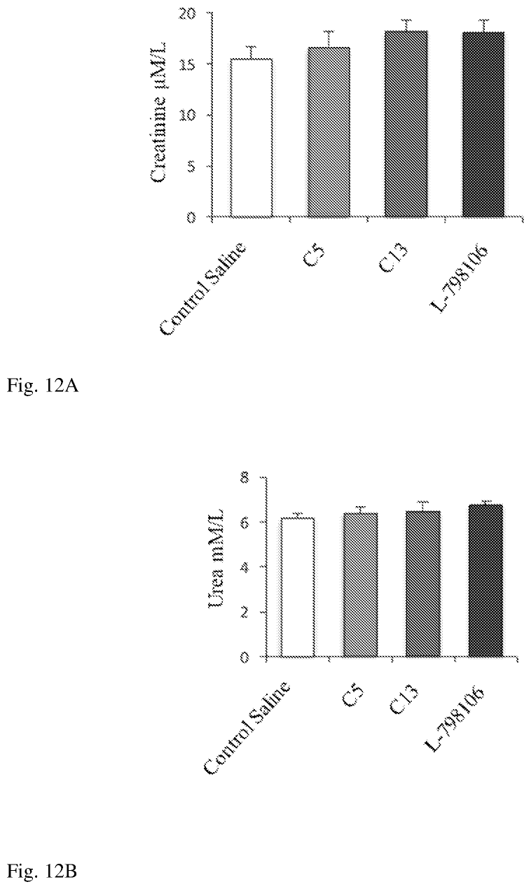

D. Effects of orally administered compounds on kidney function parameters: Creatinine and Urea levels were measured in the sera obtained from mice 24 h after the third gavage with saline/DMSO (Control Saline); 3 mg/kg/day Compound C5; Compound C13 or the EP3 antagonist L-798106. No statistically significant differences were observed for each test between the different groups, all values >0.05 as shown in FIG. 12A for Creatinine, and in For. 12 B for Urea.

### IP Administration

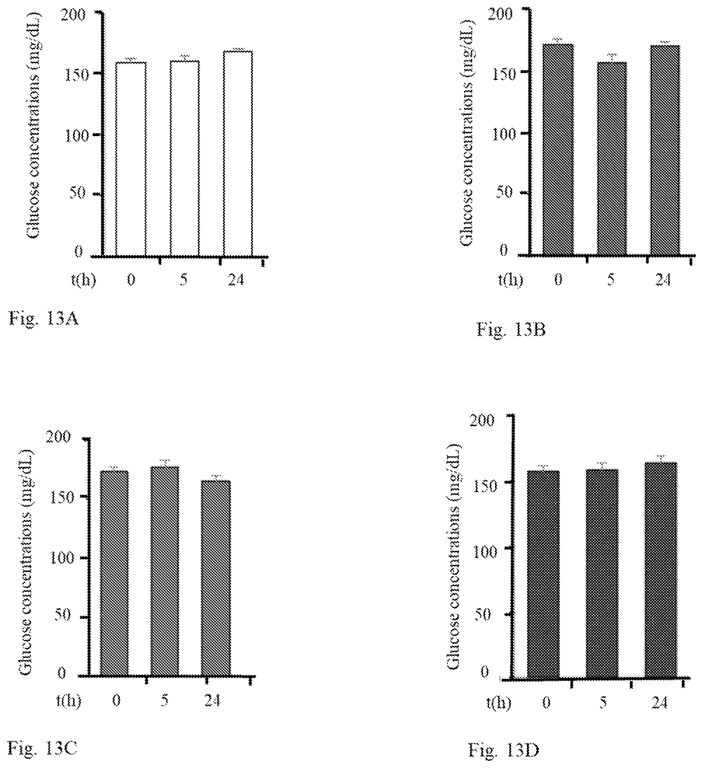

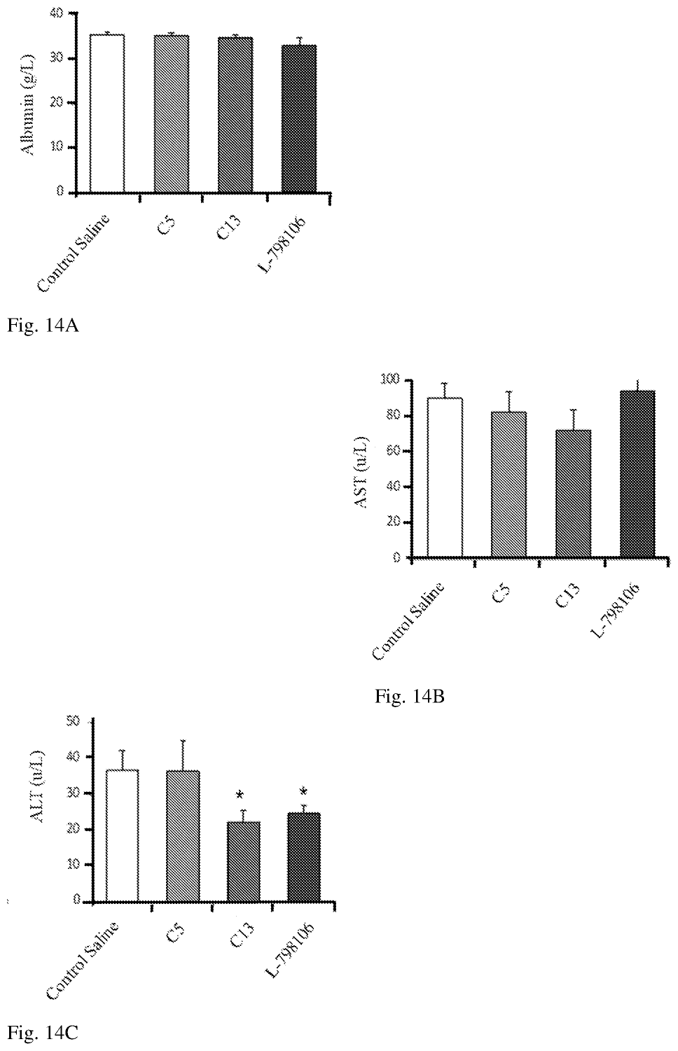

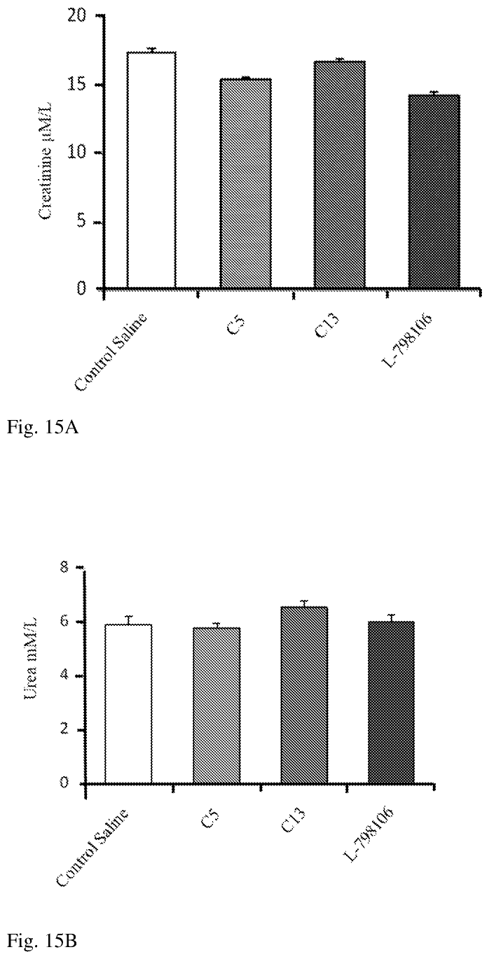

A. Glycemia evaluation: Glucose concentration was measured in blood obtained from the vein tail of C57Bl/6 mice (n=12/group). Results are represented in FIGS. 13A-13D, in which, t(h) represents time in hours as follows: before injection (t0), at time 5 h (t5) and 24 h (t24) after injection of 200 μl saline/DMSO (FIG. 13A); of 3 mg/kg of C5 (FIG. 13B); C13 (FIG. 13C); or L-798106 (FIG. 13D), by using Accu-Chek performa glucometer. As shown in FIG. 13A-13D, no differences were observed within each group and between the two experimental groups compared to control.

B. Blood insulin levels: Insulin was measured in sera obtained from mice injected with either Saline/DMSO (control), Compound 5 (C5); Compound C13 (C13) or L-798106, using ELISA mouse insulin kit according to the manufacturer's instructions (Mercodia). Five samples randomly chosen from each group were measured. As shown in FIG. 10B, no statistically significant differences were observed between the experimental groups compared to control. Similarly, to normal blood glucose, the mice keep their blood insulin concentrations within the normal range.

C. Liver function parameters: Blood levels of albumin (FIG. 14A), liver transaminases: aspartate transaminase (AST, FIG. 14B) and alanine transaminase (ALT, FIG. 14C), as well as bilirubin, were measured in sera obtained from mice 24 h after injection of either Saline/DMSO (Control Saline); C5; C13 or L-798106. As shown in FIG. 14A-14C, no statistically significant differences were observed for each test between the two experimental groups and the control group, except for the mice injected with the EP3 antagonist L-798106 or C13 which show lower concentrations of alanine transaminase (ALT). Typically, this biomarker levels rise with liver injury and help monitor function under drugs treatment. Here a decrease in the levels of ALT in the serum of L-798106- and C13-treated mice, is not considered to indicate liver damage. Bilirubin<1 μM was measured in all test groups.

D. Kidney function parameters: Creatinine (FIG. 15A) and Urea (FIG. 15B) were measured in sera obtained from mice, 24 h after injection of either Saline/DMSO (Control Saline), Compound C5 or L-798106. As shown in FIG. 15A-15B, no statistically significant differences were observed for each test between the different groups, all values >0.05.

In summary, using either mode of administration (IP or gavage) of high concentrations, namely 3 mg/kg/mouse or 9 mg/kg/mouse, respectively, of L-798106 or the tested C5 and C13 compounds, no significant differences were observed between the experimental groups and the saline/DMSO control mice. No physical or clinical behavioral differences were observed among the groups.

Further, the presented results indicate that the C5 compound did not cause abnormal liver or kidney functions as measured by representative parameters in the serum of treated animals.

### Example 8. EP3γ is Preferentially Upregulated by Palmitate and Specific EP3γ siRNA is Associated with Rescue of MIN6 Cells from Palmitate-Mediated Apoptosis

Three alternatively spliced C-terminal tails of mouse EP3 receptor, designated mEP3a, mEP30, and mEP37, further add to the diversity of the signaling pathways of PGE2-EP3 depending on the cell type. To identify which of the subtypes may be associated with palmitate-induced β cell apoptosis, RNA was extracted from palmitate-treated or untreated MIN6 cells and the subtypes were analyzed by real-time PCR.

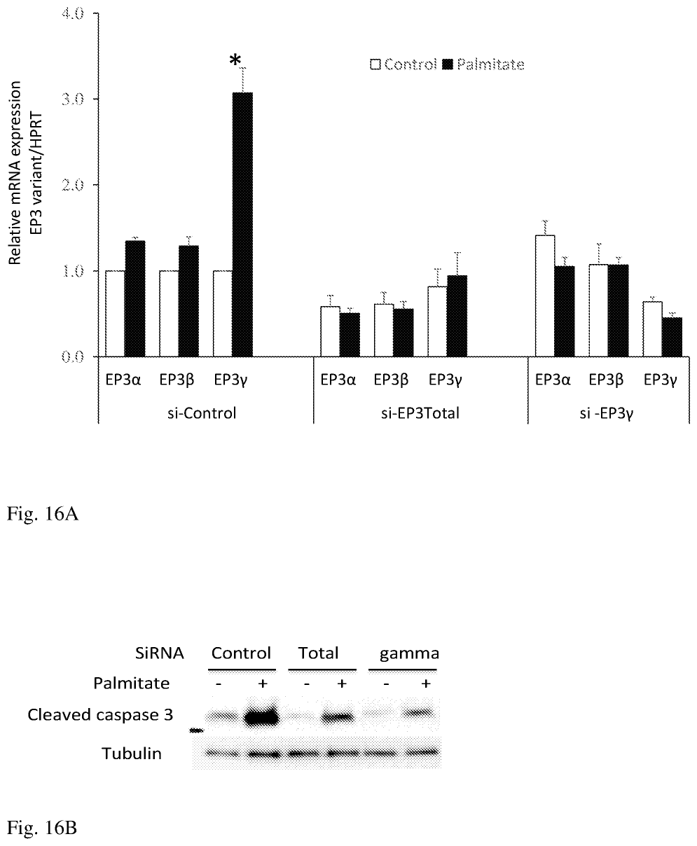

To this end, MIN6 cells were transfected with siRNAs to total EP3, specific EP3 subtypes or negative control siRNA by electroporation using Amaxa nucleofector reagent and Amaxa nucleoporator. After 24 h, the cells were treated with 0.3 mM palmitate for an additional 24 hrs. Real time PCR analysis of EP3 mRNA subtypes expression levels normalized to GAPDH was performed. The results, presented in FIG. 16A represent an average of 5 experiments ±SEM. Further presented in FIG. 16 B are the results of a Western blot analysis of cleaved Caspase 3, and α-tubulin expression levels.

As can be seen in FIG. 16, murine EP37 (mEP37) was significantly upregulated by palmitate compared to the other subtypes. As can further be seen, specific siRNA to mEP37 induced 75% decrease in the elevated mRNA levels (n=5). As can further be seen, the downregulation of mEP37 was associated with a significant decrease in apoptosis as demonstrated by the cleaved caspase-3 fragment intensity compared to the use of siRNA control ±palmitate.

While the murine EP3 gene generates EP3 splice variants, α, β, and γ, in humans EP3 is more complex with 9 spliced variants. To evaluate their levels of expression in RNA of human islets isolated from non-diabetic donors, corresponding primers were designed and tested by quantitative real-time PCR. The presence of 6 of the variants at different levels of expression (I, II, III, IV, VI and f) was detected. Sequence similarity analysis revealed that the mouse mEP3α and mEP3γ are homologous to the human variants hEP-I (EP₃-1) and hEP3-II (EP₃-2), respectively. It is therefore conceivable that the observed involvement of EP3γ to palmitate-induced β-cell apoptosis could be conserved between mouse and human. It was then evaluated whether the EP3-II gene homologous to EP3γ is altered in T2DM islets. The mRNA levels of this variant were measured by real time PCR. It was found that the EP3-II variant is significantly upregulated in T2DM human islets (n=6) compared to controls (n=7).

It was also observed that exposure of MIN6 cells to palmitate led to the upregulation of the proinflammatory gene iNOS and the chemokines IP-10, Rantes, MCP-1 and the cytokine IL-6, and inhibition of the EP3-γ pathway led to a dramatic downregulation of the detrimental genes including that of the pro-apoptotic genes (Bid, Bak) and restores the expression of the anti-apoptotic gene Bcl2 compared to the effect of si-control.

### Example 9. Effects of the Compounds C5 and C13 on Random Blood Glucose Levels in Diabetic Mice Compared to Controls

The effects of the compounds were tested in an in vivo model of diabetes. To this end, C57BLKS/J mice with the leptin receptor mutation (BKS-db; BKS.Cg-Dock7m+/+Leprdb/J), known as an animal model characteristic of human T2DM, were used, when both male and female homozygotes to Leprb mutation (db/db) develop spontaneous T2DM with phenotypes that include: (I) severe obesity, (II) progressive and sustained hyperglycemia, when the course of the disease is markedly influenced by genetic background BLKS (in contrast to regular C57BL/6J background mice with the same mutation (B6-db), (III) glucose intolerance resulting in elevated serum cholesterol, glucose, and triglycerides, and (VI) elevations of plasma blood sugar resulting in severe depletion of the insulin-producing β-cells of the pancreatic islets, and eventually death.

Two sets of experiments with two colonies of homozygote mice were performed with the C5 and C13 compounds (n=5 for each experiment), compared to L-798106. The compounds were administered to 7-8-week-old mice by oral gavage (2.5 mg/kg/day) for eight weeks. Control group (n=10-15) received vehicle (DMSO in PBS). Blood glucose was randomly tested throughout the experiment.

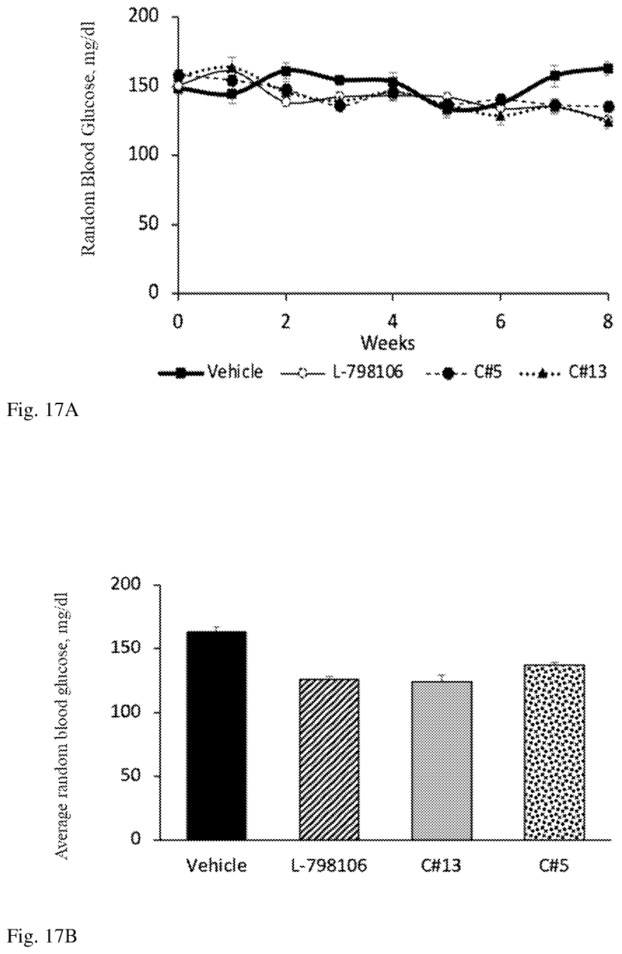

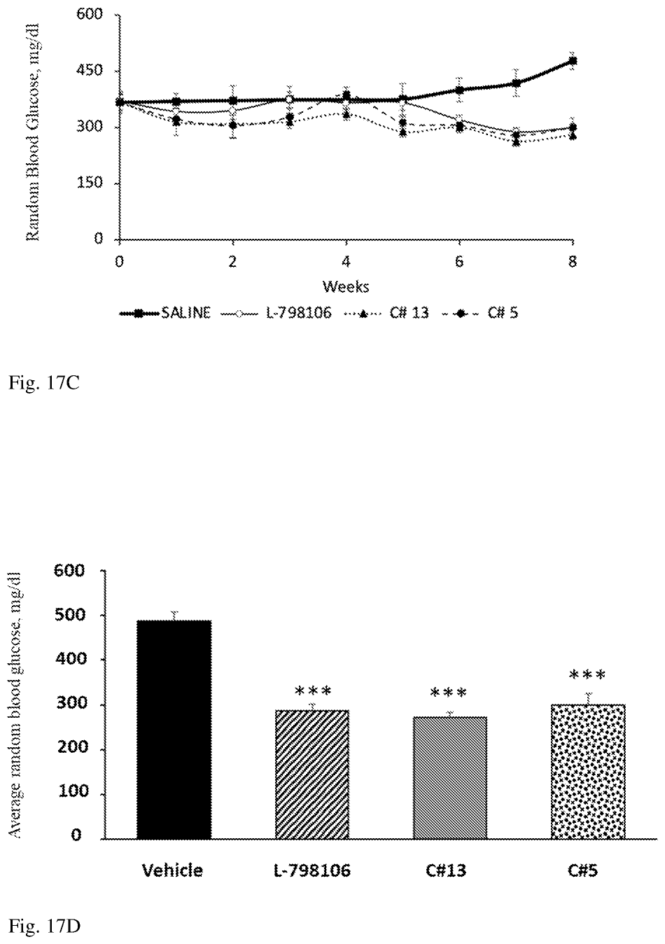

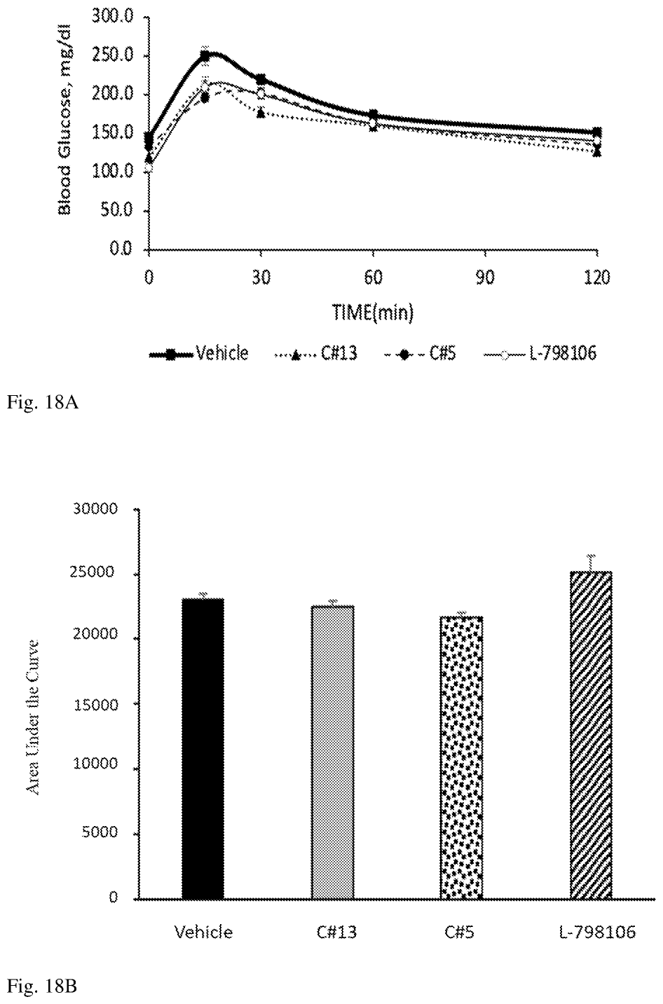

No striking physical or behavioural differences were observed between the different groups. Pictures of the mice were taken when animals were sacrificed and no damage was observed in 120 of the 121-treated mice. The results of the random blood glucose measurements are presented in FIGS. 17A-17D.

In FIGS. 17A-17D, Vehicle is represented by black squares and thick lines or black bars (n=15 for the heterozygotes/wild type, n=10 for the diabetics db/db), L-798106 is represented by open empty circles and thin black thin lines or striped bars (n=13 for wild type, n=10 for db/db), the compound C5 appears as C #5 and is represented by full circles and dashed lines or black and white spotted bars (n=10 for wild type, n=10 for db/db), and the compound C13 appears as C #13 and is represented by triangles and dotted lines or gray bars (n=10 for wild type, n=10 for db/db). *P<0.05 **p<0.01. ***p<0.001.

As can be seen in FIGS. 17A-17D, the results show a progressive decrease with time in the level of blood glucose randomly tested in the db/db mice that received the compounds (FIGS. 17C-17D) as compared to the group that received the vehicle throughout the gavage period. In comparison, the random blood glucose levels of the control mice remained substantially stable (FIGS. 17A-17B).

### Example 10. The Compounds C5 and C13 Ameliorate Glycemia In Vivo

Lepr^{db/db} homozygotes (db/db) and heterozygote Lepr^{db/+} (wild type) mice were treated daily with 2.5 mg/kg/day oral administration of the Vehicle, the commercial EP3 antagonist L-798106 and the compounds C5 and C13 for 8 weeks, as described in Example 9. A glucose tolerance test (Intraperitoneal glucose tolerance test, IPGTT) was performed following four weeks and eight weeks of the onset of treatment, as follows. Glucose (1 g/kg) was injected following 8 hours of fasting, and blood glucose was measured at baseline and at 30-, 60-, 90- and 120-minutes post glucose administration.

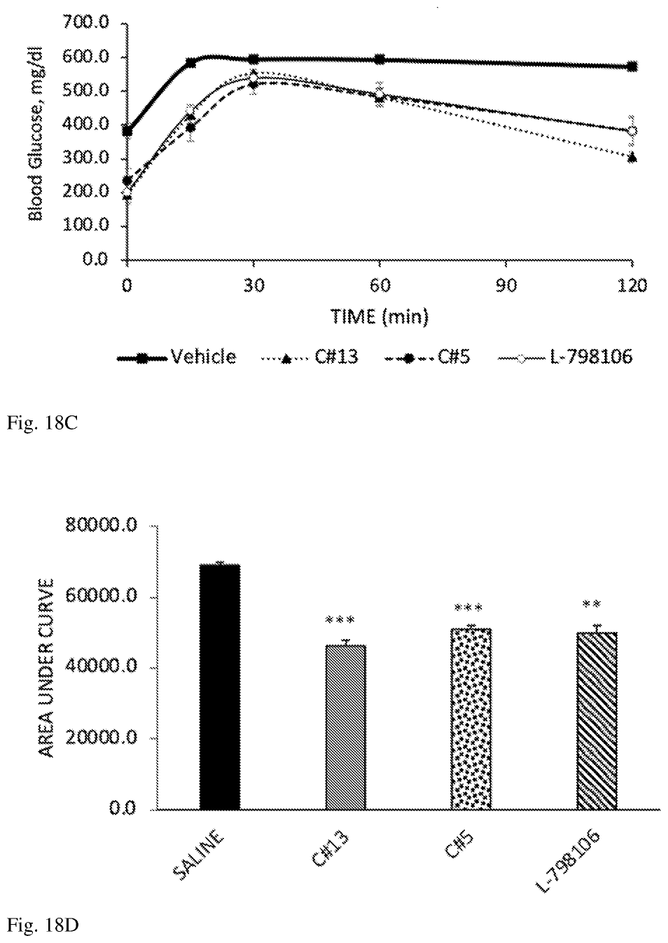

FIGS. 18A-18D show the results obtained after eight weeks of treatment, wherein blood glucose levels and area under the curve (AUC) of the wild type are presented in FIGS. 18A and 18B, respectively, and blood glucose levels and AUC of the db/db mice are presented in FIGS. 18C and 18D, respectively. The different test groups as presented in FIGS. 18A-18D are represented as described in Example 9.

As can be seen in FIGS. 18C-18D, both test compounds C5 and C13 as well as L-798106 significantly alleviated hyperglycemia in db/db mice at all time points compared to vehicle-treated mice. The reduction of hyperglycemia could already be seen following four weeks of treatment.

### Example 11. Compounds C5 and C13 Improve Insulin Sensitivity In Vivo

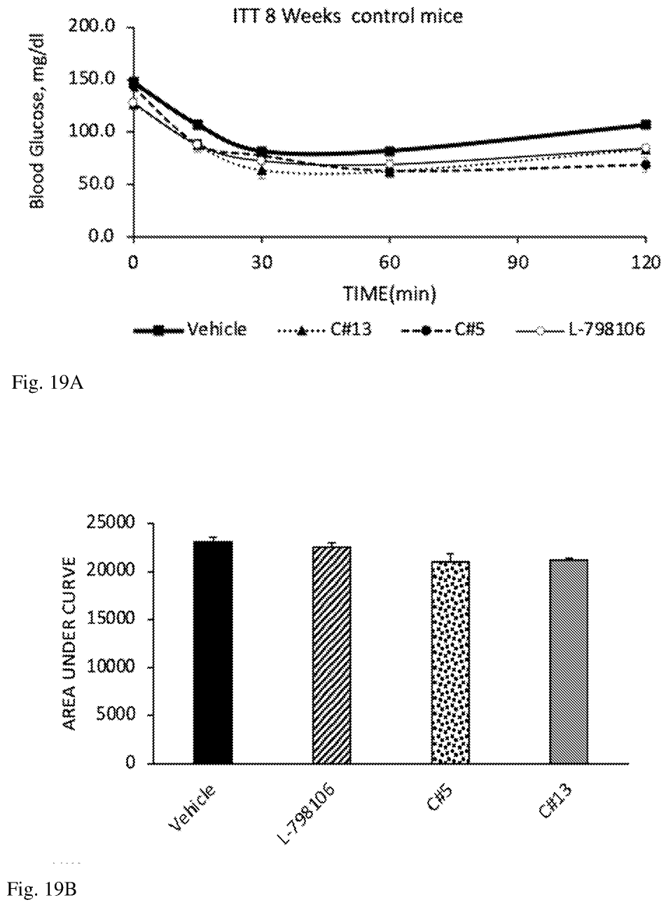

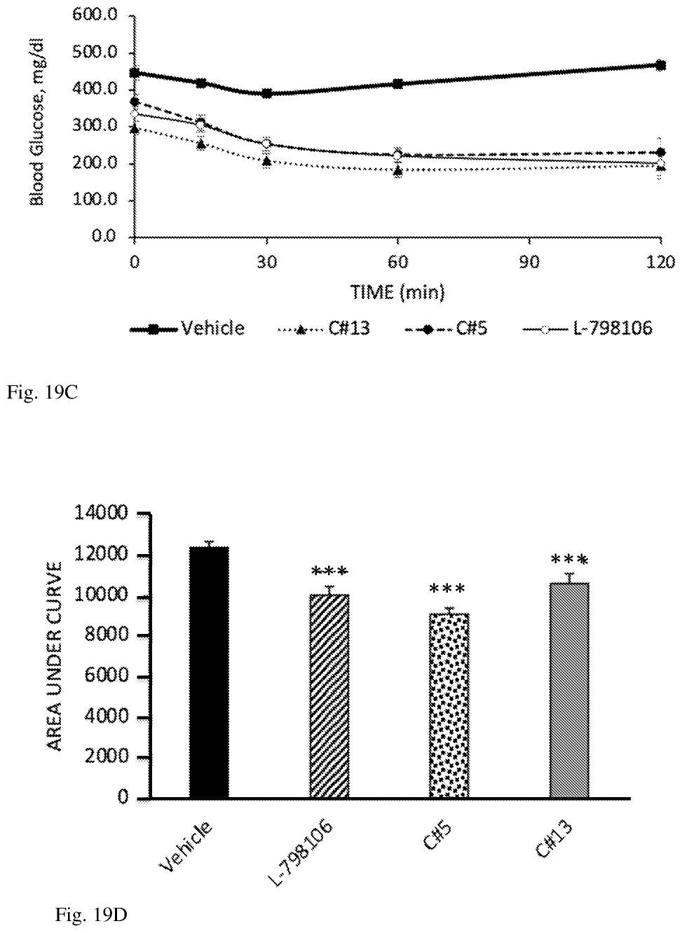

An Insulin Tolerance Test (ITT) was conducted to determine the effect of the test compounds on insulin resistance, following oral administration of Vehicle or the tested compounds as described in Example 9. To this end, the different treated groups were further subjected to a subsequent 5-hour fast and then injected with 0.75 U/kg insulin. Blood glucose was measured at the time of insulin injection (baseline level) and at the indicated time intervals thereafter. Blood glucose levels and AUC of the wild-type mice are presented in FIGS. 19A-19B respectively, and blood glucose levels and AUC of the db/db mice are presented in FIGS. 19C-19D respectively. The different test groups as presented in FIGS. 19A-19D are represented as described in Example 9.

A significant decrease in glucose levels, indicating increased insulin sensitivity of the peripheral tissues (such as muscle, fat and/or liver), was measured in the db/db mice treated by the test compounds or L-798106, as compared to the glucose levels obtained in the vehicle-treated control diabetic mice. This effect was observed at all time points following insulin injection (FIGS. 19C-19D). In addition, mice treated by the test compounds or L-798106 also showed reduced baseline glucose levels, which is in line with the results presented in FIGS. 17A-17D.

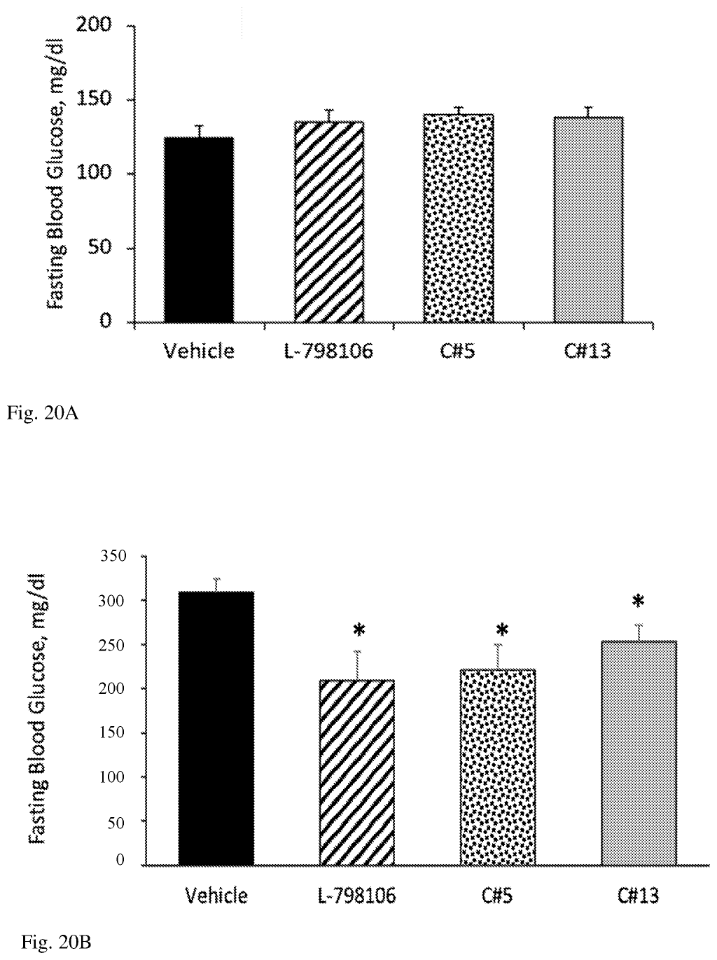

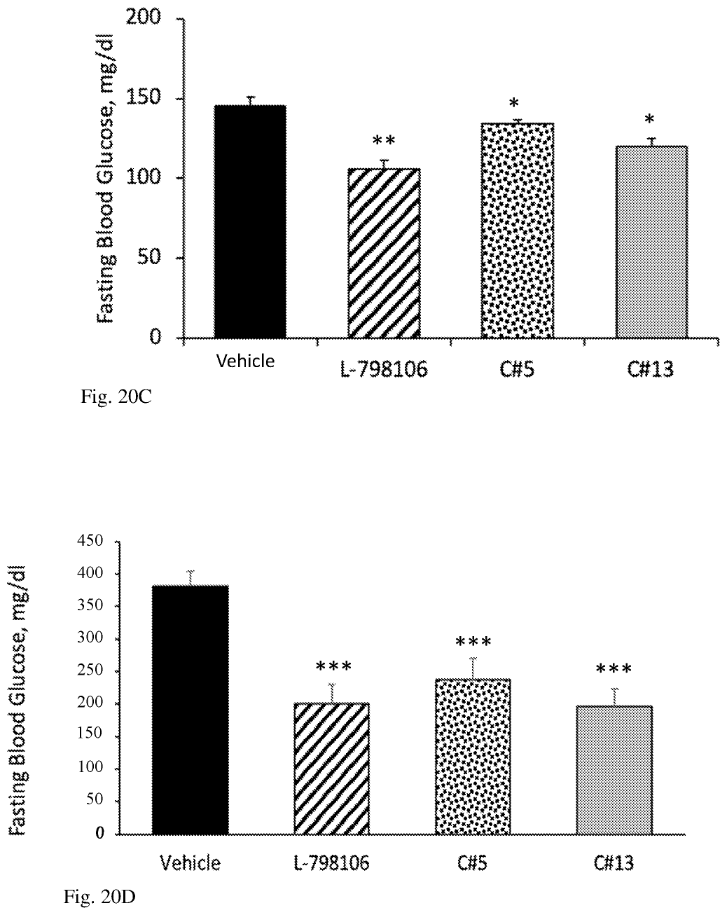

Fasting blood glucose (FBG) concentrations in each of the treated groups were also compared following 4- and 8-weeks treatment as described in Example 9. Results for the 4-weeks treatment of the control mice and the db/db mice are presented in FIGS. 20A-20B respectively and for the 8-weeks treatment of the control mice and the db/db mice are presented in FIGS. 20C-20D, respectively; each treatment is graphically represented as described in Example 9.

As can be seen in FIGS. 20A-20D, a significant decrease in FBG was already observed after 4 weeks of treatment with L-798106, C5 and C13 as compared to the vehicle in db/db mice, but not in the control mice. The effect was even more pronounced following 8 weeks of treatment; at this time point, the compounds also exerted a decrease in FBG in the control mice, although to a much lesser extent as compared to the effect in diabetic mice.

### Example 12. Blood Analyte Levels in db/db Mice Following Eight Weeks of Compounds Administration

At the end of the 8 week-long period of treatment as described in Example 9, mice were sacrificed and blood samples were collected, and analyzed for different parameters as detailed below.

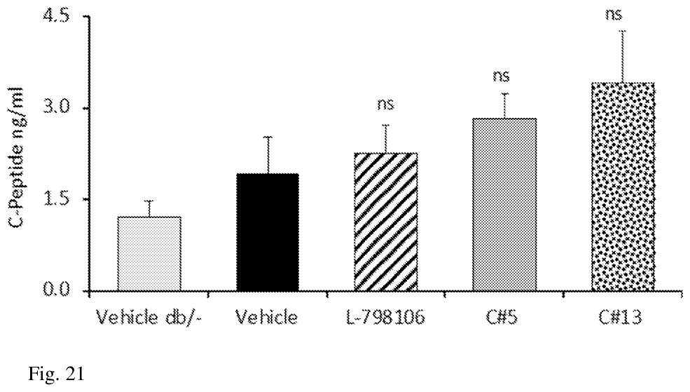

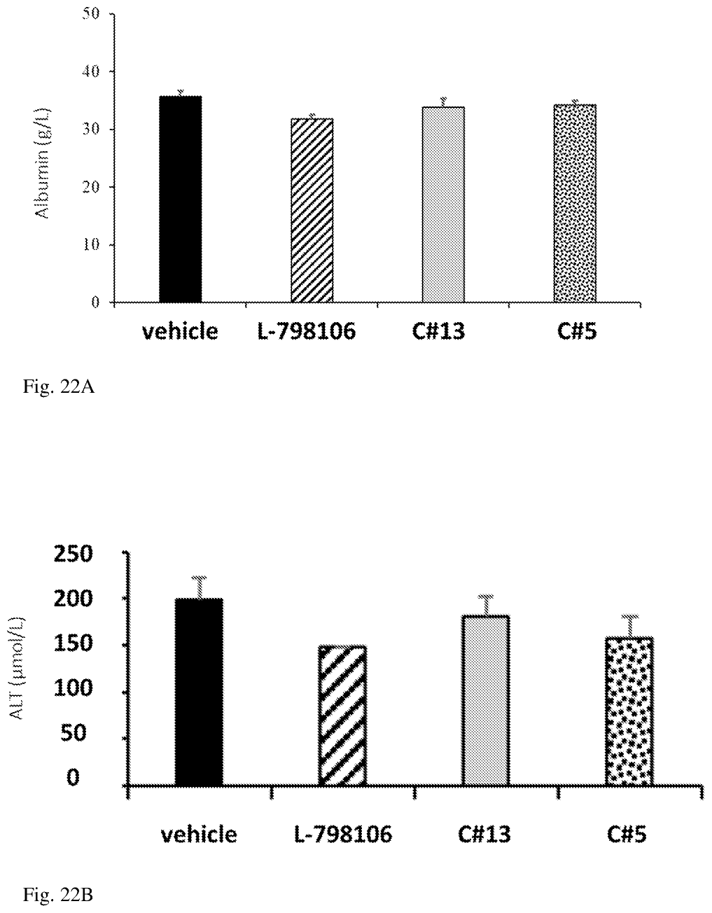

Blood insulin concentrations. The levels of insulin in the collected sera were assessed by measuring the circulating insulin substrate, C-peptide, using ELISA (Mouse C-Peptide ELISA Kit, Crystal Chem. The results are presented in FIG. 21, in which “Vehicle db/−” represents vehicle-treated control mice, “Vehicle” represents vehicle-treated diabetic mice, and “L-798106”, “C #5” and “C #13” represent diabetic mice treated with L-798106, C5 and C13, respectively. n=10 for all tested groups, and “ns” indicates non-significant differences. While the results, presented in FIG. 21, indicate slightly higher insulin levels in compounds-treated diabetic mice, the differences did not reach statistical significance.

Liver parameters in sera. Liver parameters including albumin and the liver alanine transaminase (ALT) were measured in the sera obtained from the mice following 8 weeks of treatment, as described in Example 9. Results of the albumin levels are presented in FIG. 22A and of the ALT levels in FIG. 22B, both in db/db mice. For each group FIGS. 22A-22B, n=5, data are presented ±SEM. Statistical analysis between each treated-group compared to vehicle was performed all p-values were >0.05. The results presented show no statistically significant differences for each test between the experimental groups and the control group.

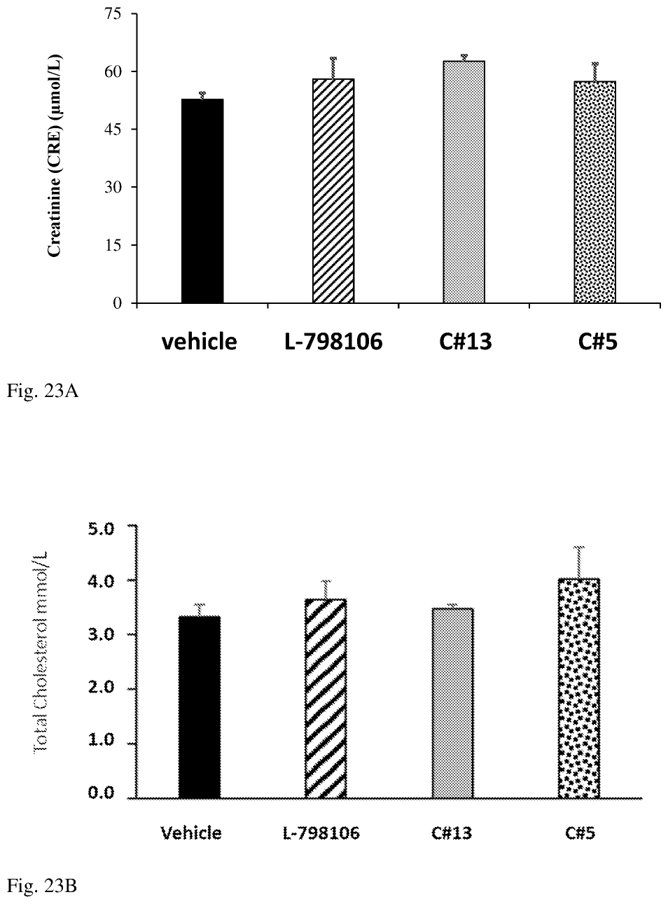

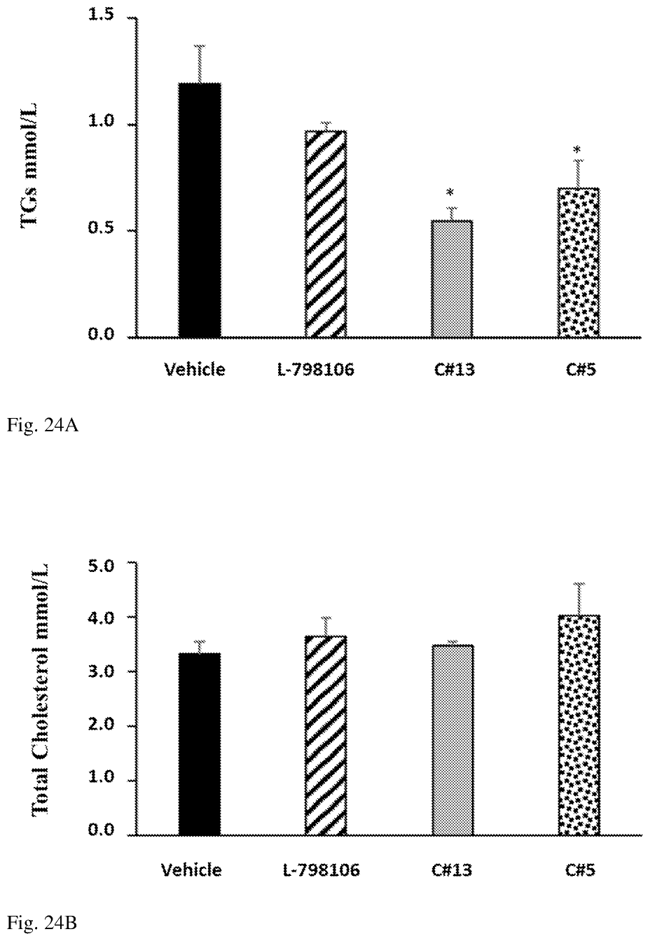

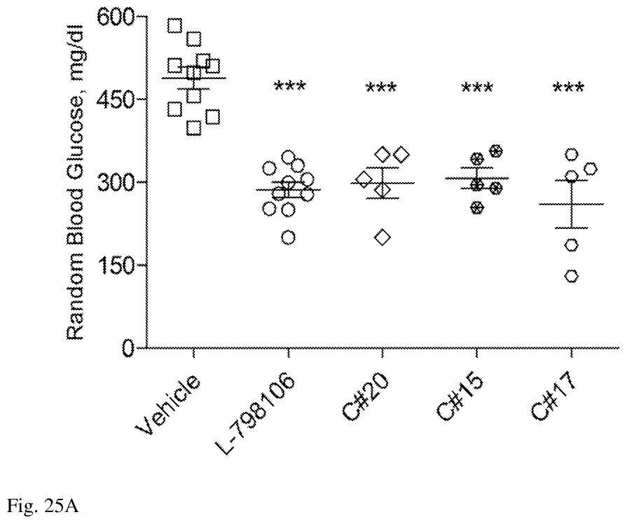

Kidney parameters in sera. Creatinine (μM/L) and Urea (mM/L) levels in the sera of db/db treated mice are presented in FIGS. 23A-B respectively, in which, each treatment is graphically represented as described in Example 9, for each group n=5, data is presented ±SEM. Statistical analysis between each treated-group compared to vehicle was performed all p-values were >0.05. Bilirubin levels were tested as well, values of all tested groups were <1 μM/L. As can be seen in FIGS. 23A-23B, no statistically significant differences were observed for each test between the different groups, all values >0.05.

Lipid parameters in sera. Mice lipid profiles were assessed by measuring triglycerides and total cholesterol levels in the sera of the treated mice. The results for the triglycerides and cholesterol levels in db/db mice are presented in FIGS. 24A-24B respectively, in which, each treatment is graphically represented as described in Example 9, for each group n=5, data is presented ±SEM. Statistical analysis between each treated-group compared to vehicle was performed p-value for C5=0.06, for C13=0.02 and p >0.05 for the rest of the tests.

As can be seen in FIG. 24A, C5 and C13-treated db/db mice exhibited significant reduction in blood triglyceride levels. In contradistinction, L-798106 did not exhibit a statistically-significant reduction. Without wishing to be bound by a specific theory or mechanism of action, this effect is advantageous at least in view of the association between high triglyceride blood levels and heart disease in the setting of T2DM. No effect on total cholesterol levels was observed between the different treated groups (FIG. 24B).

To conclude, the results presented in Examples 9-12 demonstrate that daily oral administration of compounds C5 and C13 exerted significant and remarkable therapeutic effects in a model of T2DM in vivo. In particular, these compounds were able to improve glycemia, lower fasting blood glucose, improve glucose tolerance and reduce insulin resistance. Further, these compounds were at least as effective as the commercial EP3 inhibitor, L-798106, and even exhibited improved efficacy, in particular C13. The results further demonstrate adequate safety profiles of the compounds, in both healthy and diabetic mice, upon chronic oral administration.

### Example 13. Effects of the Compounds C17 and C20 on Random Blood Glucose Levels in db/db Mice Compared to Control Mice

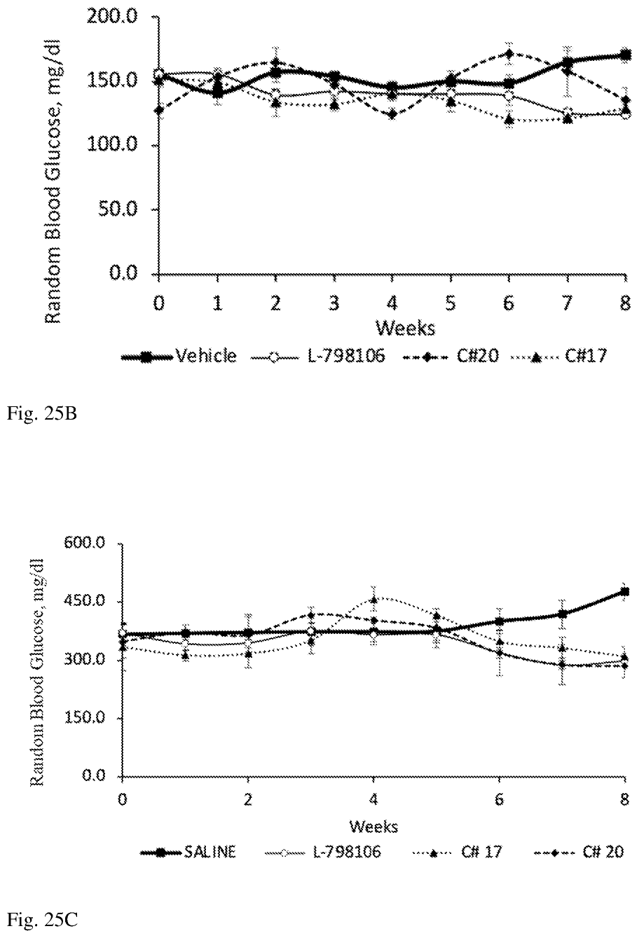

To test the effect of the compounds C15, C17 and C20 on random blood glucose levels, control and db/db mice underwent essentially the same treatment course and measurements as described in Example 9. Random blood glucose levels were measured and the results are presented in FIGS. 25A-25C. As can be seen in FIG. 25A, all three compounds, C15, C17 and C20 were determined effective in lowering average blood glucose levels in db/db mice compared to the vehicle.

FIG. 25B show the results of the random blood glucose levels over time in wild type mice, and FIG. 25C show the levels over time in db/db mice (respective averages are presented in FIG. 25A). in FIGS. 25B-25C, vehicle is represented by black rectangles and thick black lines (n=15), L-798106 is represented by empty circles and thin black lines (n=13), the compound C17 appears as C #17 and is represented by dotted line and black triangles (n=5), the compound C20 appears as C #20 and is represented by black dashed lines and black lozenges (n=8).

As can be seen in FIGS. 25B-25C, although some fluctuations were observed in blood glucose levels over time, especially for the C17 at 4 weeks, at 8 weeks, both C17 and C20 treated mice show the same blood glucose levels as the L-798106 treated mice. The results in Examples 14 and 15 below are therefore provided for compounds C17 and C20, which demonstrated the most consistent results.

### Example 14. The Compounds C17 and C20 Ameliorate Glycemia In Vivo

Heterozygote (wild type) and diabetic db/db mice were treated daily with the Vehicle, L-798106 and the compounds C17 and C20 for 8 weeks, essentially as described in Example 9. A glucose tolerance test (IPGTT) was performed essentially as described in Example 10.

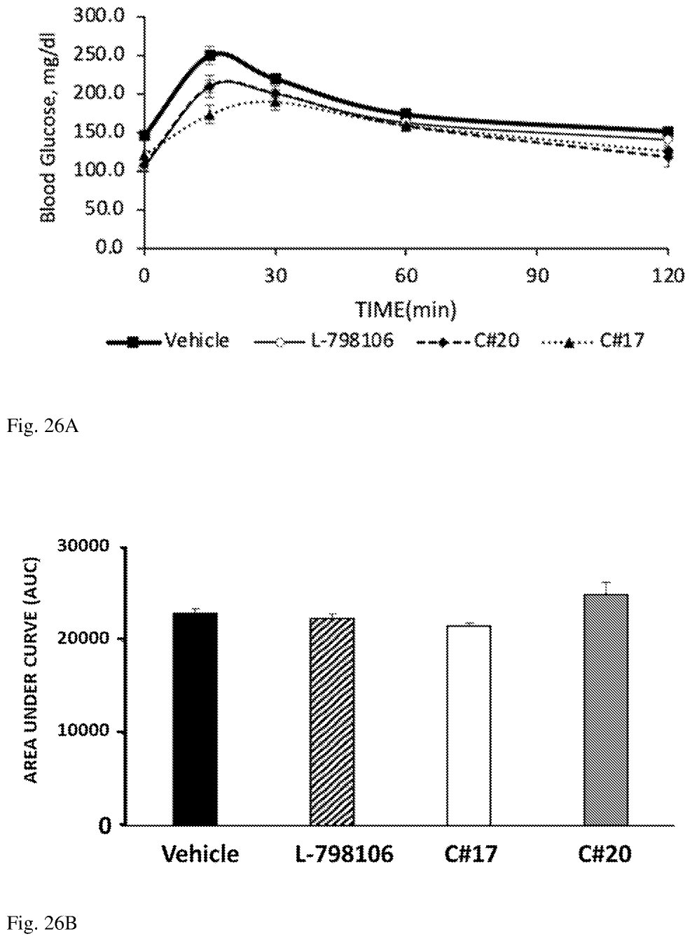

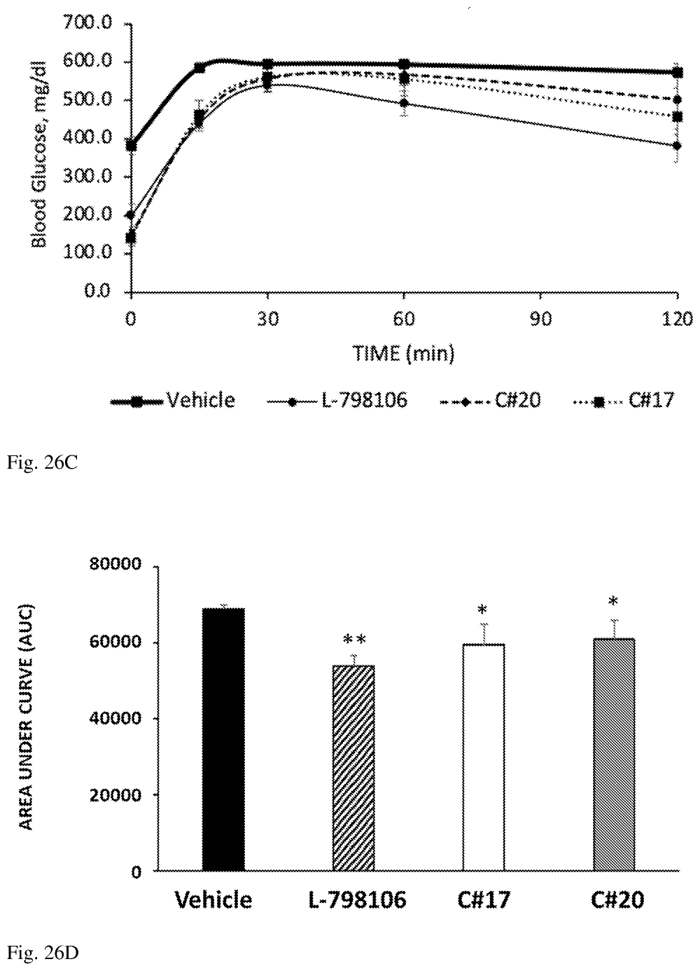

FIGS. 26A-26D show the results obtained after eight weeks of treatment, wherein blood glucose levels and AUC of the wild-type mice are presented in FIGS. 26A and 26B, respectively, and blood glucose levels and AUC of the db/db mice are presented in FIGS. 26C and 26D, respectively. The different test groups as presented in FIGS. 26A-26B are represented as described in Example 13. In addition, vehicle is represented also by black bars, L-798106 is also represented by striped bars, the compounds C17 is also represented by empty bars, and the compound C20 is also represented by gray bars.

As can be seen in FIGS. 26A-26D, both tested compounds C17 and C20 significantly alleviated hyperglycaemia in db/db mice compared to vehicle-treated mice at all time points; however, the effect of L-798106 appeared to be statistically more significant in this assay.

### Example 15. Compounds C17 and C20 Improve Insulin Sensitivity In Vivo

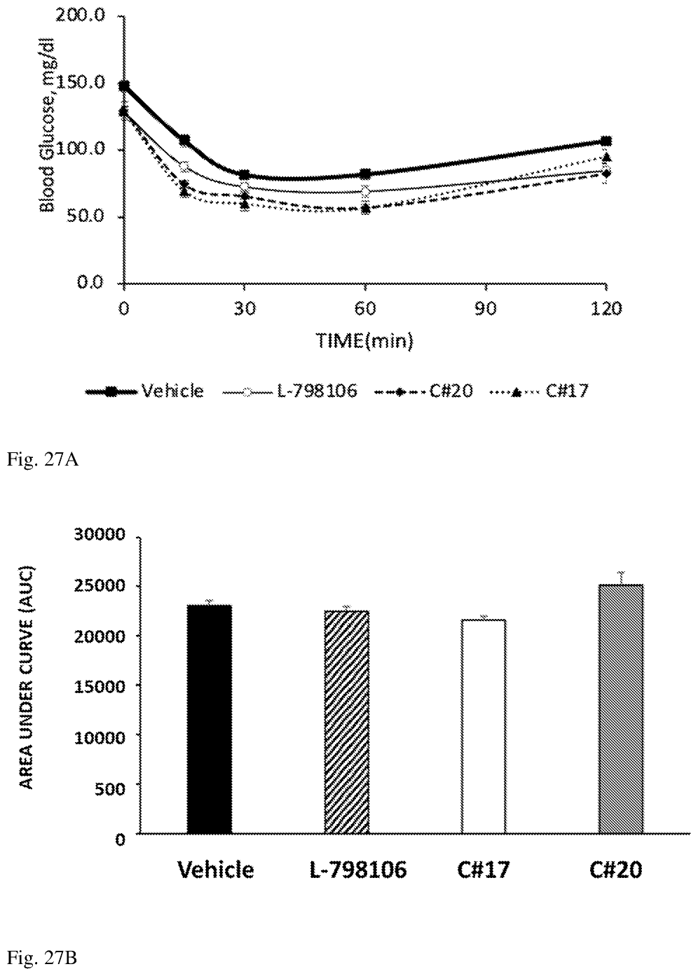

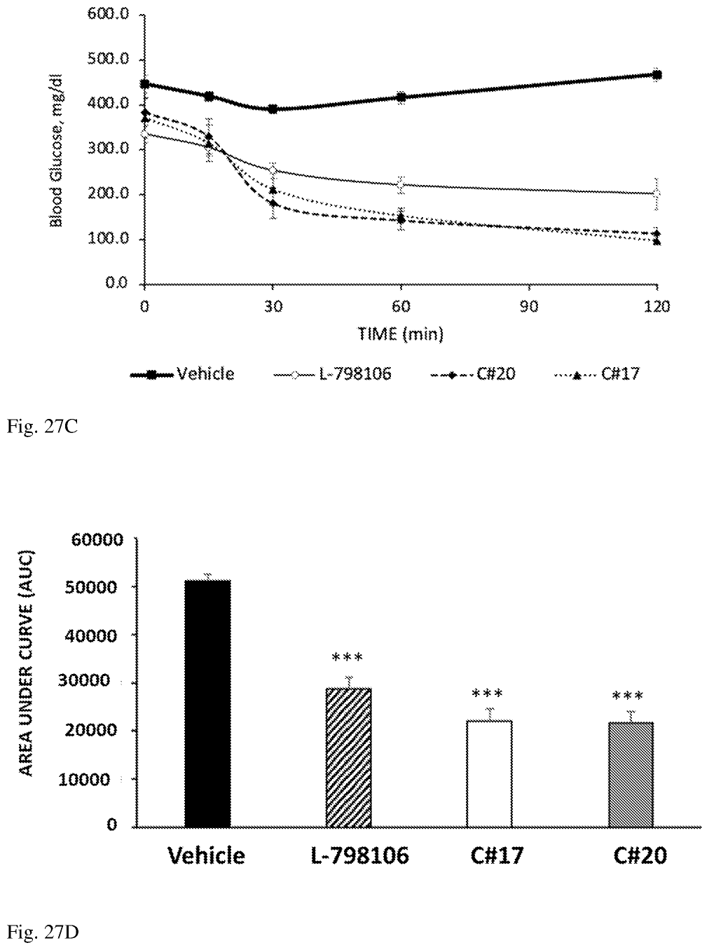

To determine the effect of the C17 and C20 compounds on insulin resistance, an ITT was conducted essentially as described in Example 11. Blood glucose levels and AUC of the wild-type mice are presented in FIGS. 27A-27B respectively (n=5-15), and blood glucose levels and AUC of the db/db mice are presented in FIGS. 27C-27D respectively (n=5-10). The different test groups as presented in FIGS. 27A-27D are represented as described in Example 14.

As can be seen in FIGS. 27C-27D, a significant decrease in glucose levels, indicating increased insulin sensitivity of the peripheral tissues (such as muscle, fat and/or liver), was measured in the db/db mice treated by the test compounds or L-798106, as compared to the glucose levels obtained in the vehicle-treated control diabetic mice. This effect was observed at all time points following insulin injection in mice treated by the test compounds for 8 weeks.

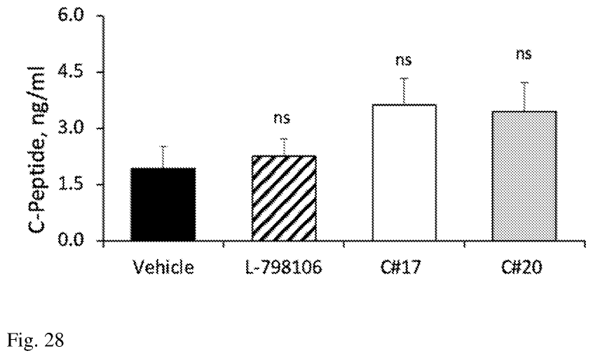

Blood insulin concentrations. Following 8 weeks of treatment, blood samples were collected and the levels of insulin in the collected sera were assessed essentially as described in Example 12. The results are presented in FIG. 28, in which the different test groups as presented in FIG. 28 are represented as described in Example 14 and “ns” indicates non-significant differences. The numbers of tested mice are as follows: vehicle (n=10), L-798106 (n=10), C17 (n=5), C20 (n=5). While the results, presented in FIG. 28, indicate slightly higher insulin levels in compounds-treated diabetic mice, the differences did not reach statistical significance.

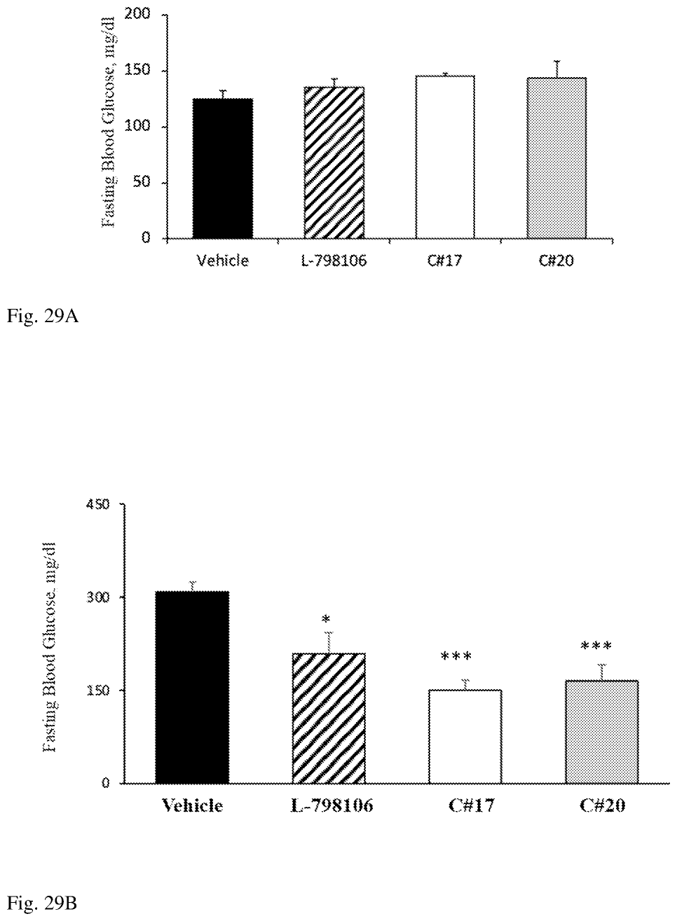

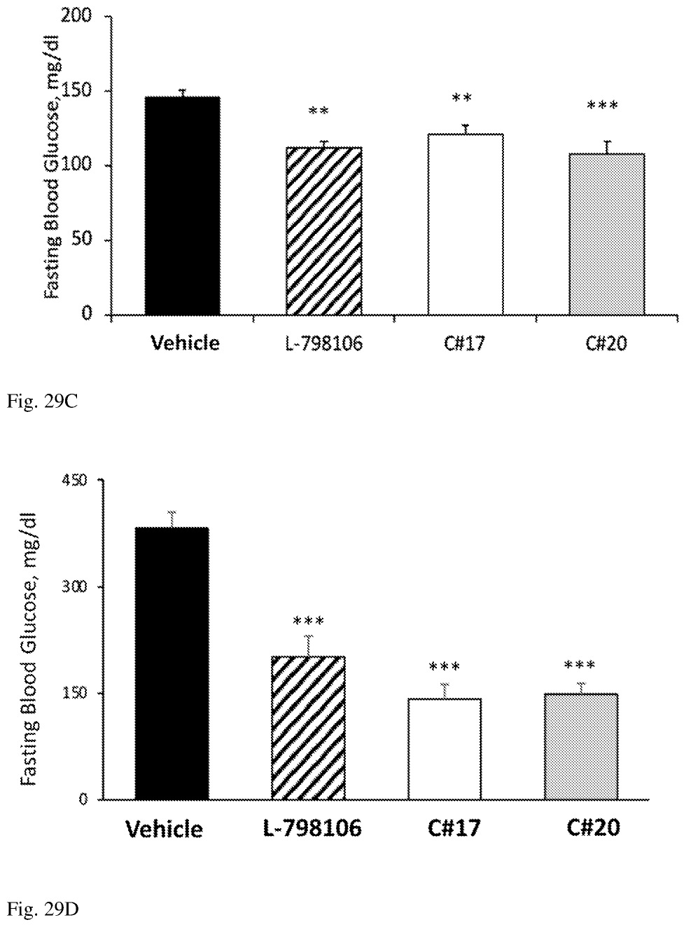

FBG concentrations. FBG concentrations in each of the treated groups, vehicle, L-798106, C17 and C20, was compared following 4- and 8-weeks treatment essentially as described in Example 11. Results for the 4-weeks treatment of the control mice and the db/db mice are presented in FIGS. 29A-29B respectively and for the 8-weeks treatment of the control mice and the db/db mice are presented in FIGS. 29C-29D, in which, each treatment is graphically represented as described in Example 14, The numbers of tested mice are as follows: control mice (n=10-15), db/db mice (n=10). As can be seen in FIGS. 29A-29B, a significant decrease in FBG is already observed after 4 weeks of treatment with L-798106, C5 and C13 as compared to the vehicle in db/db mice db/db and to the control mice. The effect is significantly more pronounced following 8 weeks of treatment (FIGS. 29C-29D).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for treating or preventing the progression of diabetes in a human subject in need thereof, comprising administering to the subject a pharmaceutical composition comprising a compound represented by a formula selected from the group consisting of:
(i) Formula I, or a salt thereof: 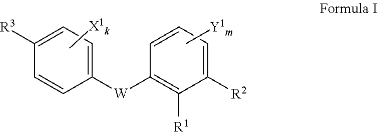
wherein
W is —NHSOₙ—, wherein n is 1 or 2;
one of R¹ and R² is CONR⁴R⁵, and the other one of R¹ and R² is hydrogen, wherein each one of R⁴ and R⁵ is independently selected from the group consisting of H, alkyl, aryl, arylalkyl, cycloalkyl and haloalkyl, or wherein R⁴ and R⁵ together with the nitrogen atom to which they are bound form a ring, wherein the formed ring is unsubstituted or substituted with at least one alkyl, halogen, or haloalkyl;
each one of X¹ and Y¹ is independently selected from the group consisting of a halogen and an alkyl;
each one of m and k is 0, 1 or 2; and
R³ is selected from hydrogen and an alkyl group;
(ii) Formula II, an ester or a salt thereof: 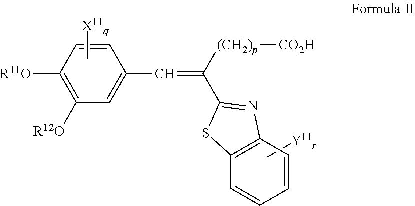
wherein
R¹¹ is CH₂R¹³, wherein R¹³ is selected from the group consisting of hydrogen, an alkyl and an aryl;
R¹² is selected from the group consisting of hydrogen and an alkyl;
each one of X¹¹ and Y¹¹ is independently selected from the group consisting of a halogen and an alkyl; and
each one of p, rand q is 0, 1 or 2; and
(iii) Formula III, an ester or a salt thereof: 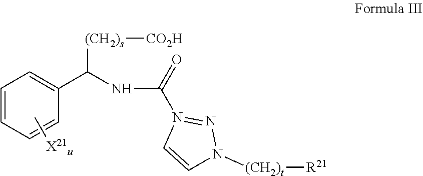
wherein
u is 1 or 2;
each X²¹ is selected from the group consisting of OH, OR²² and halogen, wherein R²² is selected from the group consisting of unsubstituted alkyl, unsubstituted aryl, haloalkyl, alkoxyalkyl, hydroxyalkyl and haloaryl;
each one of s and t is independently 0, 1 or 2; and
R²¹ is selected from the group consisting of unsubstituted aryl, haloaryl, alkoxyaryl, alkylaryl, unsubstituted heteroaryl, haloheteroaryl, alkoxyheteroaryl and alkylheteroaryl.

2. The method according to claim 1, wherein the compound is represented by Formula I or a salt thereof.

3. The method according to claim 2, wherein the compound is represented by Formula I-1, or a salt thereof: 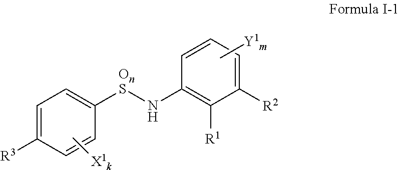
wherein R¹, R², R³, X¹, Y¹, n, m and k are as defined in claim 1.

4. The method according to claim 3, wherein R² is CONR⁴R⁵, and R¹ is hydrogen, m is 0 and n is 2, wherein R⁴ and R⁵ together with the nitrogen atom to which they are bound form a non-aromatic N-heterocycle, wherein the non-aromatic N-heterocycle is unsubstituted or substituted with one or more alkyl, halogen, or haloalkyl, and wherein R³ is a C₁-C₄ alkyl.

5-7. (canceled)

8. The method according to claim 2, wherein the compound is selected from the group consisting of Compound 1a, Compound 1b, Compound 1c and salts thereof:

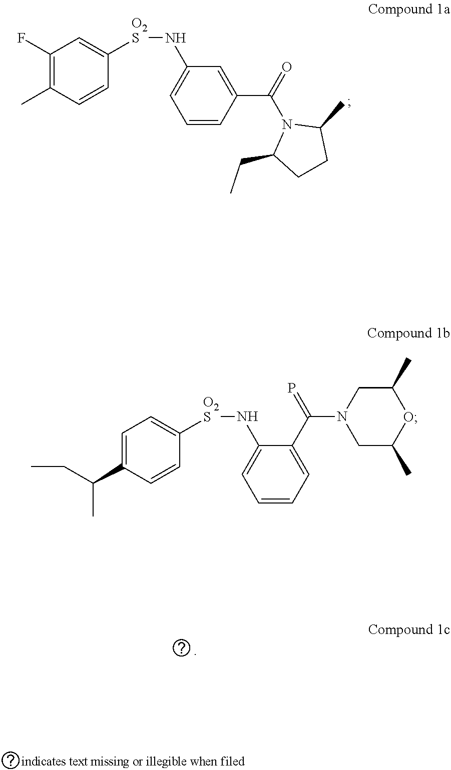

9-15. (canceled)

16. The method according to claim 1, wherein said compound is a selective prostaglandin receptor 3 (EP3) antagonist, or
wherein said compound inhibits palmitate-induced apoptosis in human islet β cells.

17. (canceled)

18. The method according to claim 1, wherein the diabetes is type II diabetes mellitus (T2DM) or type I diabetes mellitus (T1DM), and
wherein the diabetes is characterized by EP3 overexpression in pancreatic β cells.

19-21. (canceled)

22. The method according to m claim 1,
wherein administration is performed so as to prevent or inhibit loss of pancreatic β cell mass and/or activity in said subject, or
wherein administration is performed so as to prevent or reduce lipotoxicity-induced β cell damage in said subject, or
wherein administration is performed so as to enhance glucose-induced insulin secretion by pancreatic β cells in said subject.

23-24. (canceled)

25. A method of preserving or promoting the viability of pancreatic β cells, comprising contacting a population of the β cells with a compound represented by a formula selected from the group consisting of Formulae I, II and III, as defined in claim 1, esters and salts thereof, thereby preserving or promoting the viability of said β cells.

26. The method of claim 25, wherein the contacting is performed ex vivo, or
wherein the contacting is performed in vitro, or
wherein said β cells are obtained from a healthy human donor, or
wherein the cell population is selected from the group consisting of: purified primary β cells, β cell lines, genetically modified β cells, primary pancreatic islet cells, and fetal pancreatic islet cells.

27. (canceled)

28. The method of claim 25, wherein said β cells are characterized by EP3 overexpression, and
wherein said compound is used in an amount sufficient to prevent or reduce lipotoxicity-induced β cell damage.

29-31. (canceled)

32. The method of claim 25, wherein said compound is represented by Formula I-1, or a salt thereof:

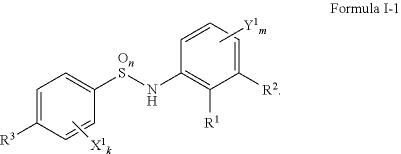

33-34. (canceled)

35. The method of claim 25, wherein the compound is selected from the group consisting of compounds Compound 1a, Compound 1b, Compound 1c, Compound 2a, Compound 2b, Compound 2c, and Compound 3 as set forth below, esters and salts thereof,

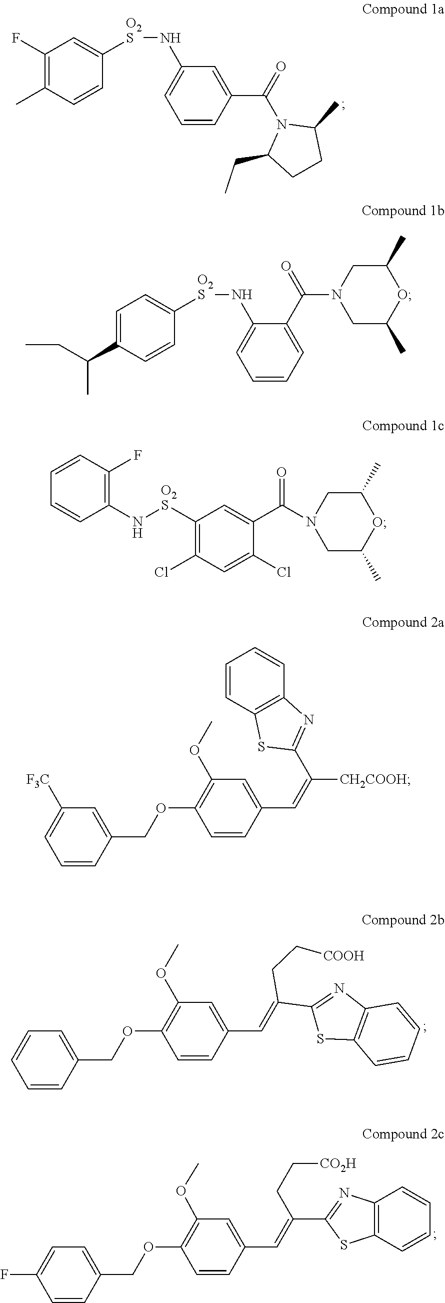

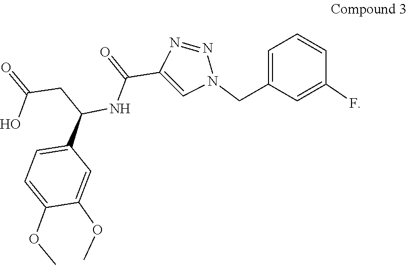 [SMILES: COc1ccc([C@@H](CC(=O)O)NC(=O)c2cn(Cc3cccc(F)c3)nn2)cc1OC]

36. The method of claim 25, wherein said compound is a selective prostaglandin receptor 3 (EP3) antagonist, and inhibits palmitate-induced apoptosis in human islet β cells.

37. (canceled)

38. The method of claim 25, wherein the β cells are from a human subject having a pancreatic β cell disorder selected from the group consisting of: pancreatic β cell failure, T2DM, T1DM, pre-diabetes, and insulin resistance, or from a human subject diagnosed with chronic hyperglycemia and dyslipidemia, and
wherein said subject further receives a pancreatic β cell transplantation, a pancreatic islet transplantation or a pancreatic transplantation.

39. (canceled)

40. The method of claim 38, wherein the contacting is performed in vivo.

41. A cell composition for pancreatic β cell transplantation or pancreatic islet transplantation, comprising the β cell population that has been contacted with said compound as described in claim 25.

42. A pharmaceutical composition comprising a pharmaceutical grade purity of one or more compounds selected from the group consisting of Compound 1a, Compound 1b, Compound 1c, Compound 2a, Compound 2b, Compound 2c, and Compound 3 as defined in claim 35, esters and salts thereof, said composition further comprising a pharmaceutically acceptable carrier, excipient or diluent.

43. The pharmaceutical composition of claim 42 comprising at least one of Compound 1a, Compound 1b and salts thereof.

44-46. (canceled)

47. A method of treating or preventing the progression of diabetes in a human subject in need thereof, or of preserving or promoting the viability of pancreatic β cells in the subject, comprising administering to said subject the pharmaceutical composition of claim 42, thereby treating or preventing the progression of diabetes in said subject.

48. (canceled)


SEQUENCE LISTING (8 sequences)

SEQ ID NO: 1 - DNA, 20 bp, Artificial Sequence
  PCR primer
cgtgttcctacccccaatgt

SEQ ID NO: 2 - DNA, 22 bp, Artificial Sequence
  PCR primer
tgcttcaccaccttcttgatgt

SEQ ID NO: 3 - DNA, 19 bp, Artificial Sequence
  PCR primer
aacccccagcccttagtga

SEQ ID NO: 4 - DNA, 21 bp, Artificial Sequence
  PCR primer
gggtaggaagtgcaccaacag

SEQ ID NO: 5 - DNA, 20 bp, Artificial Sequence
  PCR primer
acccacaagtggcacaactg

SEQ ID NO: 6 - DNA, 22 bp, Artificial Sequence
  PCR primer
gatctacaatgccacgcttctg

SEQ ID NO: 7 - DNA, 23 bp, Artificial Sequence
  PCR primer
tcccatctccttcatgacatctc

SEQ ID NO: 8 - DNA, 21 bp, Artificial Sequence
  PCR primer
gccgaggatttggaaaaagtg